SEQUENCE LISTING (31 sequences)

SEQ ID NO: 1 - RNA, 1620 nt, synthetic construct
  source 1..1620: mol_type = other RNA
atgaagatggccagcagcgacgccaacccaagcgacggcagcgcagccaacctggtgcccgaggtgaacaacgaggtgatggccctggagccagtggtgggagcagccatcgcagccccagtggcaggccagcagaacgtgatcgacccatggatccggaacaacttcgtgcaggccccaggaggcgagttcaccgtgagcccacggaacgccccaggcgagatcctgtggagcgcaccactgggcccagacctgaacccatacctgagccacctggcccggatgtacaacggctacgccggcggcttcgaggtgcaggtgatcctggccggcaacgccttcaccgcaggcaagatcatcttcgccgccgtgccacccaacttcccaaccgagggcctgagcccaagccaggtgaccatgttcccacacatcatcgtggacgtgaggcagctggagcccgtgctgatcccactgcccgacgtgcggaacaacttctaccactacaaccagagcaacgacagcaccatcaagctgatcgccatgctgtacaccccactgcgggccaacaacgccggcgacgacgtgttcaccgtgagctgcagggtgctgaccaggcccagcccagacttcgacttcatcttcctggtgccacccaccgtggagagccggaccaagcccttcagcgtgcccgtgctgaccgtggaggagatgaccaacagccggttcccaatcccactggagaagctgttcaccggaccaagcagcgccttcgtggtgcagccacagaacggccggtgcaccaccgacggagtgctgctgggcaccacccagctgagcccagtgaacatctgcaccttccggggcgacgtgacccacatcaccggcagccggaactacaccatgaacctggccagccagaactggaacaactacgacccaaccgaggagatcccagccccactgggcaccccagacttcgtgggcaagatccagggcatgctgacccagaccaccagaaccgacggcagcaccagaggccacaaggccaccgtgtacaccggcagcgccgacttcgccccaaagctgggccgggtgcagttcgagaccgacaccgaccacgacttcgaggccaaccagaacaccaagttcaccccagtgggcgtgatccaggacggcagcaccacccaccggaacgagccacagcagtgggtgctgcccagctacagcggcaggaacacccacaacgtgcacctggcaccagccgtggcaccaaccttcccaggcgagcagctgctgttcttccggagcaccatgccaggctgcagcggctacccaaacatggacctggactgcctgctgccacaggagtgggtgcagtacttctaccaggaggcagccccagcacagagcgacgtggccctgctgcggttcgtgaacccagacaccggacgggtgctgttcgagtgcaagctgcacaagagcggctacgtgaccgtggcccacaccggacagcacgacctggtgatcccacccaacggctacttccggttcgacagctgggtgaaccagttctacaccctggccccaatgggcaacggaaccggacggaggagggtggtg

SEQ ID NO: 2 - RNA, 1590 nt, synthetic construct
  source 1..1590: mol_type = other RNA
atgatgatggccagcaaggacgccaccagcagcgtggacggagccagcggcgcaggacagctggtgcccgaggtgaacgccagcgacccactggccatggacccagtggccggaagcagcaccgcagtggccaccgcaggccaggtgaacccaatcgacccatggatcatcaacaacttcgtgcaggccccacagggcgagttcaccatcagcccaaacaacaccccaggcgacgtgctgttcgacctgagcctgggcccacacctgaacccattcctgctgcacctgagccagatgtacaacggctgggtgggcaacatgcgggtgcggatcatgctggccggcaacgccttcaccgccggcaagatcatcgtgagctgcatcccacccggcttcggcagccacaacctgaccatcgcccaggccaccctgttcccacacgtgatcgccgacgtgcggaccctggacccaatcgaggtgccactggaggacgtgcggaacgtgctgttccacaacaacgaccggaaccagcagaccatgcggctggtgtgcatgctgtacaccccactgcggaccggaggcggaaccggagacagcttcgtggtggccggccgggtgatgacctgcccaagcccagacttcaacttcctgttcctggtgccacccaccgtggagcagaagacccggcccttcaccctgcccaacctgccactgagcagcctgagcaacagccgggccccactgcccatcagcagcatgggcatcagcccagacaacgtgcagagcgtgcagttccagaacggccggtgcaccctggacggacggctggtgggcaccaccccagtgagcctgagccacgtggccaagatccggggcaccagcaacggcaccgtgatcaacctgaccgagctggacggcaccccattccacccattcgagggcccagccccaatcggcttcccagacctgggcggctgcgactggcacatcaacatgacccagttcggccacagcagccagacccagtacgacgtggacaccaccccagacaccttcgtgccacacctgggcagcatccaggccaacggcatcggcagcggcaactacgtgggcgtgctgagctggatcagcccacccagccacccaagcggcagccaggtggacctgtggaagatcccaaactacggcagcagcatcaccgaggccacccacctggccccaagcgtgtacccacccggcttcggcgaggtgctggtgttcttcatgagcaagatgcccggcccaggcgcctacaacctgccctgcctgctgccacaggagtacatcagccacctggccagcgagcaggccccaaccgtgggcgaggcagccctgctgcactacgtggacccagacaccggccggaacctgggcgagttcaaggcctacccagacggcttcctgacctgcgtgcccaacggagccagcagcggcccacagcagctgcccatcaacggcgtgttcgtgttcgtgagctgggtgagccggttctaccagctgaagcccgtgggaaccgccagcagcgccagaggacggctgggactgaggcgg

SEQ ID NO: 3 - RNA, 1641 nt, synthetic construct
  source 1..1641: mol_type = other RNA
atgaagatggccagcaacgacgccgccccaagcaacgacggagccgccaacctggtgcccgaggccaccaacgaggtgatggccctggagcccgtggtgggagccagcatcgcagccccagtggtgggccagcagaacatcatcgacccatggatccgggagaacttcgtgcaggccccacagggcgagttcaccgtgagcccacggaacagcccaggcgagatgctgctgaacctggagctgggcccagagctgaacccatacctgggccacctgagccggatgtacaacggctacgccggcggcatgcaggtgcaggtggtgctggccggcaacgccttcaccgccggcaagatcatcttcgccgccgtgccaccacacttcccagtggagaacatcagcgccgcccagatcaccatgtgcccacacgtgatcgtggacgtgcggcagctggagcccgtgctgctgccactgcccgacatccggaaccggttcttccactacaaccaggagaacaccccacggatgcggctggtggccatgctgtacaccccactgcgggccaacagcggcgaggacgtgttcaccgtgagctgccgggtgctgaccaggccagccccagacttcgagttcaccttcctggtgccacccaccgtggagagcaagaccaagcccttcaccctgcccatcctgaccctgggcgagctgagcaacagccggttcccagccccaatcgacatgctgtacaccgacccaaacgaggccatcgtggtgcagccacagaacggccggtgcaccctggacggcaccctgcagggcaccacccagctggtgcccacccagatctgcagcttccggggcaccctgatcagccagaccagccggagcgcagacagcaccgacagcgccccacgggtgcggaaccacccactgcacgtgcagctgaagaacctggacggcaccccatacgacccaaccgacgaggtgcccgcagtgctgggcgccatcgacttcaagggcaccgtgttcggcatcgccagccagcggaacaccaccggcagcagcatcggagccacccgggcacacgaggtgcacatcgacaccaccaacccacggtacaccccaaagctgggcagcgtgctgatgtacagcgagagcaacgacttcgacgacggccagcccacccggttcaccccaatcggcatgggcgccgacgactggcggcagtgggagctgcccgagtacagcggccacctgaccctgaacatgaacctggccccagccgtggcaccagccttcccaggcgagcggatcctgttcttccggagcgtggtgcccagcgcaggaggctacggcagcggccacatcgactgcctgatcccacaggagtgggtgcagcacttctaccaggaggccgcaccaagccagagcgccgtggccctgatccggtacgtgaacccagacaccggccggaacatcttcgaggccaagctgcaccgggagggcttcatcaccgtggccaacagcggcaacaacccaatcgtggtgccacccaacggctacttccggttcgaggcctgggtgaaccagttctacaccctgaccccaatgggcaccggacagggacggaggcgggtgcag

SEQ ID NO: 4 - RNA, 1626 nt, synthetic construct
  source 1..1626: mol_type = other RNA
atgaagatggccagcaacgacgccgcaccaagcaccgacggagccgcaggactggtgcccgagagcaacaacgaggtgatggccctggagcccgtggcaggagccgcactggcagcaccagtgaccggccagaccaacatcatcgacccatggatccgggccaacttcgtgcaggccccaaacggcgagttcaccgtgagcccacggaacgccccaggcgaggtgctgctgaacctggagctgggcccagagctgaacccatacctggcccacctggcccggatgtacaacggctacgccggcggcatggaggtgcaggtgatgctggccggcaacgccttcaccgccggcaagctggtgttcgccgccgtgccaccacacttcccagtggagaacctgagcccacagcagatcaccatgttcccacacgtgatcatcgacgtgcggaccctggagcccgtgctgctgccactgcccgacgtgcggaacaacttcttccactacaaccagaaggacgacccaaagatgcggatcgtggccatgctgtacaccccactgcggagcaacggcagcggcgacgacgtgttcaccgtgagctgccgggtgctgaccaggcccagcccagacttcgacttcacctacctggtgccacccaccgtggagagcaagaccaagcccttcaccctgcccatcctgaccctgggcgagctgagcaacagccggttcccagtgagcatcgaccagatgtacaccagcccaaacgagatcatcagcgtgcagtgccagaacggccggtgcaccctggacggcgagctgcagggcaccacccagctgcaggtgagcggcatctgcgccttcaagggcgaggtgaccgcccacctgcacgacaacgaccacctgtacaacgtgaccatcaccaacctgaacggcagcccattcgacccaagcgaggacatcccagccccactgggcgtgcccgacttccagggccgggtgttcggcatcatcagccagcgggacaagcacaacagcccaggccacaacgagcccgccaaccggggacacgacgccgtggtgcccacctacaccgcccagtacaccccaaagctgggccagatccagatcggcacctggcagaccgacgacctgaccgtgaaccagcccgtgaagttcaccccagtgggcctgaacgacaccgagcacttcaaccagtgggtggtgccacggtacgccggcgccctgaacctgaacaccaacctggccccaagcgtggccccagtgttcccaggcgagcggctgctgttcttccggagctacatcccactgaagggcggctacggcaacccagccatcgactgcctgctgccacaggagtgggtgcagcacttctaccaggaggccgccccaagcatgagcgaggtggccctggtgcggtacatcaacccagacaccggccgggccctgttcgaggccaagctgcaccgggccggcttcatgaccgtgagcagcaacaccagcgccccagtggtggtgcccgccaacggctacttccggttcgacagctgggtgaaccagttctacagcctggccccaatgggcaccggcaacggacggaggcgggtgcag

SEQ ID NO: 5 - RNA, 1644 nt, synthetic construct
  source 1..1644: mol_type = other RNA
atgaagatggccagcaacgacgccaccccaagcaacgacggagccgcaggcctggtgcccgagatcaacaacgaggccatggccctggagcccgtggcaggagcagccatcgccgcaccactgaccggccagcagaacatcatcgacccatggatcatgaacaacttcgtgcaggccccaggcggcgagttcaccgtgagcccacggaacagcccaggcgaggtgctgctgaacctggagctgggcccagagatcaacccatacctggcccacctggcccggatgtacaacggctacgccggcggcttcgaggtgcaggtggtgctggccggcaacgccttcaccgccggcaagatcatcttcgccgccatcccacccaacttcccaatcgacaacctgagcgccgcccagatcaccatgtgcccacacgtgatcgtggacgtgcggcagctggagcccgtgaacctgcccatgcccgacgtgcggaacaacttcttccactacaaccagggaagcgacagccggctgcggctggtggccatgctgtacaccccactgcgggccaacaacagcggcgacgacgtgttcaccgtgagctgccgggtgctgaccaggcccagcccagagttcagcttcaacttcctggtgccacccaccgtggagagcaagaccaagcccttcaccctgcccatcctgaccatcagcgagatgagcaacagccggttcccagtgcccatcgacagcctgcacaccagcccaaccgagaacatcgtggtgcagtgccagaacggccgggtgaccctggacggcgagctgatgggcaccacccagctgctgcccagccagatctgcgccttccggggagtgctgacccggagcaccagccgggcaagcgaccaggcagacaccgccaccccacggctgttcaactactactggcacatccagctggacaacctgaacggcaccccatacgacccagccgaggacatcccaggcccactgggcaccccagacttccggggcaaggtgttcggcgtggccagccagcggaacccagacagcaccacccgggcccacgaggccaaggtggacaccaccgccggccggttcaccccaaagctgggcagcctggagatcagcaccgagagcgacgacttcgaccagaaccagcccacccggttcaccccagtgggcatcggcgtggacaacgaggccgacttccagcagtggagcctgcccgactacagcggccagttcacccacaacatgaacctggccccagccgtggccccaaacttcccaggcgagcagctgctgttcttccggagccagctgcccagcagcggaggacggagcaacggcatcctggactgcctggtgccacaggagtgggtgcagcacttctaccaggagagcgcaccagcccagacccaggtggccctggtgcggtacgtgaacccagacaccggccgggtgctgttcgaggccaagctgcacaagctgggcttcatgaccatcgccaagaacggcgacagcccaatcaccgtgccacccaacggctacttccggttcgagagctgggtgaacccattctacaccctggccccaatgggcaccggcaacggacggaggcgggtgcag

SEQ ID NO: 6 - RNA, 47 nt, synthetic construct
  source 1..47: mol_type = other RNA
  modified_base 1: mod_base = OTHER; note = 2'-O-Methyl modified nucleotide
aggaaataagagagaaaagaagagtaagaagaaatataagagccacc

SEQ ID NO: 7 - RNA, 194 nt, synthetic construct
  source 1..194: mol_type = other RNA
gcggccgcttaattaagctgccttctgcggggcttgccttctggccatgcccttcttctctcccttgcacctgtacctcttggtctttgaataaagcctgagtaggaagtctagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - RNA, 173 nt, synthetic construct
  source 1..173: mol_type = other RNA
gctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - DNA, 1620 bp, synthetic construct
  source 1..1620: mol_type = other DNA
atgaagatggccagcagcgacgccaacccaagcgacggcagcgcagccaacctggtgcccgaggtgaacaacgaggtgatggccctggagccagtggtgggagcagccatcgcagccccagtggcaggccagcagaacgtgatcgacccatggatccggaacaacttcgtgcaggccccaggaggcgagttcaccgtgagcccacggaacgccccaggcgagatcctgtggagcgcaccactgggcccagacctgaacccatacctgagccacctggcccggatgtacaacggctacgccggcggcttcgaggtgcaggtgatcctggccggcaacgccttcaccgcaggcaagatcatcttcgccgccgtgccacccaacttcccaaccgagggcctgagcccaagccaggtgaccatgttcccacacatcatcgtggacgtgaggcagctggagcccgtgctgatcccactgcccgacgtgcggaacaacttctaccactacaaccagagcaacgacagcaccatcaagctgatcgccatgctgtacaccccactgcgggccaacaacgccggcgacgacgtgttcaccgtgagctgcagggtgctgaccaggcccagcccagacttcgacttcatcttcctggtgccacccaccgtggagagccggaccaagcccttcagcgtgcccgtgctgaccgtggaggagatgaccaacagccggttcccaatcccactggagaagctgttcaccggaccaagcagcgccttcgtggtgcagccacagaacggccggtgcaccaccgacggagtgctgctgggcaccacccagctgagcccagtgaacatctgcaccttccggggcgacgtgacccacatcaccggcagccggaactacaccatgaacctggccagccagaactggaacaactacgacccaaccgaggagatcccagccccactgggcaccccagacttcgtgggcaagatccagggcatgctgacccagaccaccagaaccgacggcagcaccagaggccacaaggccaccgtgtacaccggcagcgccgacttcgccccaaagctgggccgggtgcagttcgagaccgacaccgaccacgacttcgaggccaaccagaacaccaagttcaccccagtgggcgtgatccaggacggcagcaccacccaccggaacgagccacagcagtgggtgctgcccagctacagcggcaggaacacccacaacgtgcacctggcaccagccgtggcaccaaccttcccaggcgagcagctgctgttcttccggagcaccatgccaggctgcagcggctacccaaacatggacctggactgcctgctgccacaggagtgggtgcagtacttctaccaggaggcagccccagcacagagcgacgtggccctgctgcggttcgtgaacccagacaccggacgggtgctgttcgagtgcaagctgcacaagagcggctacgtgaccgtggcccacaccggacagcacgacctggtgatcccacccaacggctacttccggttcgacagctgggtgaaccagttctacaccctggccccaatgggcaacggaaccggacggaggagggtggtg

SEQ ID NO: 10 - DNA, 1590 bp, synthetic construct
  source 1..1590: mol_type = other DNA
atgatgatggccagcaaggacgccaccagcagcgtggacggagccagcggcgcaggacagctggtgcccgaggtgaacgccagcgacccactggccatggacccagtggccggaagcagcaccgcagtggccaccgcaggccaggtgaacccaatcgacccatggatcatcaacaacttcgtgcaggccccacagggcgagttcaccatcagcccaaacaacaccccaggcgacgtgctgttcgacctgagcctgggcccacacctgaacccattcctgctgcacctgagccagatgtacaacggctgggtgggcaacatgcgggtgcggatcatgctggccggcaacgccttcaccgccggcaagatcatcgtgagctgcatcccacccggcttcggcagccacaacctgaccatcgcccaggccaccctgttcccacacgtgatcgccgacgtgcggaccctggacccaatcgaggtgccactggaggacgtgcggaacgtgctgttccacaacaacgaccggaaccagcagaccatgcggctggtgtgcatgctgtacaccccactgcggaccggaggcggaaccggagacagcttcgtggtggccggccgggtgatgacctgcccaagcccagacttcaacttcctgttcctggtgccacccaccgtggagcagaagacccggcccttcaccctgcccaacctgccactgagcagcctgagcaacagccgggccccactgcccatcagcagcatgggcatcagcccagacaacgtgcagagcgtgcagttccagaacggccggtgcaccctggacggacggctggtgggcaccaccccagtgagcctgagccacgtggccaagatccggggcaccagcaacggcaccgtgatcaacctgaccgagctggacggcaccccattccacccattcgagggcccagccccaatcggcttcccagacctgggcggctgcgactggcacatcaacatgacccagttcggccacagcagccagacccagtacgacgtggacaccaccccagacaccttcgtgccacacctgggcagcatccaggccaacggcatcggcagcggcaactacgtgggcgtgctgagctggatcagcccacccagccacccaagcggcagccaggtggacctgtggaagatcccaaactacggcagcagcatcaccgaggccacccacctggccccaagcgtgtacccacccggcttcggcgaggtgctggtgttcttcatgagcaagatgcccggcccaggcgcctacaacctgccctgcctgctgccacaggagtacatcagccacctggccagcgagcaggccccaaccgtgggcgaggcagccctgctgcactacgtggacccagacaccggccggaacctgggcgagttcaaggcctacccagacggcttcctgacctgcgtgcccaacggagccagcagcggcccacagcagctgcccatcaacggcgtgttcgtgttcgtgagctgggtgagccggttctaccagctgaagcccgtgggaaccgccagcagcgccagaggacggctgggactgaggcgg

SEQ ID NO: 11 - DNA, 1641 bp, synthetic construct
  source 1..1641: mol_type = other DNA
atgaagatggccagcaacgacgccgccccaagcaacgacggagccgccaacctggtgcccgaggccaccaacgaggtgatggccctggagcccgtggtgggagccagcatcgcagccccagtggtgggccagcagaacatcatcgacccatggatccgggagaacttcgtgcaggccccacagggcgagttcaccgtgagcccacggaacagcccaggcgagatgctgctgaacctggagctgggcccagagctgaacccatacctgggccacctgagccggatgtacaacggctacgccggcggcatgcaggtgcaggtggtgctggccggcaacgccttcaccgccggcaagatcatcttcgccgccgtgccaccacacttcccagtggagaacatcagcgccgcccagatcaccatgtgcccacacgtgatcgtggacgtgcggcagctggagcccgtgctgctgccactgcccgacatccggaaccggttcttccactacaaccaggagaacaccccacggatgcggctggtggccatgctgtacaccccactgcgggccaacagcggcgaggacgtgttcaccgtgagctgccgggtgctgaccaggccagccccagacttcgagttcaccttcctggtgccacccaccgtggagagcaagaccaagcccttcaccctgcccatcctgaccctgggcgagctgagcaacagccggttcccagccccaatcgacatgctgtacaccgacccaaacgaggccatcgtggtgcagccacagaacggccggtgcaccctggacggcaccctgcagggcaccacccagctggtgcccacccagatctgcagcttccggggcaccctgatcagccagaccagccggagcgcagacagcaccgacagcgccccacgggtgcggaaccacccactgcacgtgcagctgaagaacctggacggcaccccatacgacccaaccgacgaggtgcccgcagtgctgggcgccatcgacttcaagggcaccgtgttcggcatcgccagccagcggaacaccaccggcagcagcatcggagccacccgggcacacgaggtgcacatcgacaccaccaacccacggtacaccccaaagctgggcagcgtgctgatgtacagcgagagcaacgacttcgacgacggccagcccacccggttcaccccaatcggcatgggcgccgacgactggcggcagtgggagctgcccgagtacagcggccacctgaccctgaacatgaacctggccccagccgtggcaccagccttcccaggcgagcggatcctgttcttccggagcgtggtgcccagcgcaggaggctacggcagcggccacatcgactgcctgatcccacaggagtgggtgcagcacttctaccaggaggccgcaccaagccagagcgccgtggccctgatccggtacgtgaacccagacaccggccggaacatcttcgaggccaagctgcaccgggagggcttcatcaccgtggccaacagcggcaacaacccaatcgtggtgccacccaacggctacttccggttcgaggcctgggtgaaccagttctacaccctgaccccaatgggcaccggacagggacggaggcgggtgcag

SEQ ID NO: 12 - DNA, 1626 bp, synthetic construct
  source 1..1626: mol_type = other DNA
atgaagatggccagcaacgacgccgcaccaagcaccgacggagccgcaggactggtgcccgagagcaacaacgaggtgatggccctggagcccgtggcaggagccgcactggcagcaccagtgaccggccagaccaacatcatcgacccatggatccgggccaacttcgtgcaggccccaaacggcgagttcaccgtgagcccacggaacgccccaggcgaggtgctgctgaacctggagctgggcccagagctgaacccatacctggcccacctggcccggatgtacaacggctacgccggcggcatggaggtgcaggtgatgctggccggcaacgccttcaccgccggcaagctggtgttcgccgccgtgccaccacacttcccagtggagaacctgagcccacagcagatcaccatgttcccacacgtgatcatcgacgtgcggaccctggagcccgtgctgctgccactgcccgacgtgcggaacaacttcttccactacaaccagaaggacgacccaaagatgcggatcgtggccatgctgtacaccccactgcggagcaacggcagcggcgacgacgtgttcaccgtgagctgccgggtgctgaccaggcccagcccagacttcgacttcacctacctggtgccacccaccgtggagagcaagaccaagcccttcaccctgcccatcctgaccctgggcgagctgagcaacagccggttcccagtgagcatcgaccagatgtacaccagcccaaacgagatcatcagcgtgcagtgccagaacggccggtgcaccctggacggcgagctgcagggcaccacccagctgcaggtgagcggcatctgcgccttcaagggcgaggtgaccgcccacctgcacgacaacgaccacctgtacaacgtgaccatcaccaacctgaacggcagcccattcgacccaagcgaggacatcccagccccactgggcgtgcccgacttccagggccgggtgttcggcatcatcagccagcgggacaagcacaacagcccaggccacaacgagcccgccaaccggggacacgacgccgtggtgcccacctacaccgcccagtacaccccaaagctgggccagatccagatcggcacctggcagaccgacgacctgaccgtgaaccagcccgtgaagttcaccccagtgggcctgaacgacaccgagcacttcaaccagtgggtggtgccacggtacgccggcgccctgaacctgaacaccaacctggccccaagcgtggccccagtgttcccaggcgagcggctgctgttcttccggagctacatcccactgaagggcggctacggcaacccagccatcgactgcctgctgccacaggagtgggtgcagcacttctaccaggaggccgccccaagcatgagcgaggtggccctggtgcggtacatcaacccagacaccggccgggccctgttcgaggccaagctgcaccgggccggcttcatgaccgtgagcagcaacaccagcgccccagtggtggtgcccgccaacggctacttccggttcgacagctgggtgaaccagttctacagcctggccccaatgggcaccggcaacggacggaggcgggtgcag

SEQ ID NO: 13 - DNA, 1644 bp, synthetic construct
  source 1..1644: mol_type = other DNA
atgaagatggccagcaacgacgccaccccaagcaacgacggagccgcaggcctggtgcccgagatcaacaacgaggccatggccctggagcccgtggcaggagcagccatcgccgcaccactgaccggccagcagaacatcatcgacccatggatcatgaacaacttcgtgcaggccccaggcggcgagttcaccgtgagcccacggaacagcccaggcgaggtgctgctgaacctggagctgggcccagagatcaacccatacctggcccacctggcccggatgtacaacggctacgccggcggcttcgaggtgcaggtggtgctggccggcaacgccttcaccgccggcaagatcatcttcgccgccatcccacccaacttcccaatcgacaacctgagcgccgcccagatcaccatgtgcccacacgtgatcgtggacgtgcggcagctggagcccgtgaacctgcccatgcccgacgtgcggaacaacttcttccactacaaccagggaagcgacagccggctgcggctggtggccatgctgtacaccccactgcgggccaacaacagcggcgacgacgtgttcaccgtgagctgccgggtgctgaccaggcccagcccagagttcagcttcaacttcctggtgccacccaccgtggagagcaagaccaagcccttcaccctgcccatcctgaccatcagcgagatgagcaacagccggttcccagtgcccatcgacagcctgcacaccagcccaaccgagaacatcgtggtgcagtgccagaacggccgggtgaccctggacggcgagctgatgggcaccacccagctgctgcccagccagatctgcgccttccggggagtgctgacccggagcaccagccgggcaagcgaccaggcagacaccgccaccccacggctgttcaactactactggcacatccagctggacaacctgaacggcaccccatacgacccagccgaggacatcccaggcccactgggcaccccagacttccggggcaaggtgttcggcgtggccagccagcggaacccagacagcaccacccgggcccacgaggccaaggtggacaccaccgccggccggttcaccccaaagctgggcagcctggagatcagcaccgagagcgacgacttcgaccagaaccagcccacccggttcaccccagtgggcatcggcgtggacaacgaggccgacttccagcagtggagcctgcccgactacagcggccagttcacccacaacatgaacctggccccagccgtggccccaaacttcccaggcgagcagctgctgttcttccggagccagctgcccagcagcggaggacggagcaacggcatcctggactgcctggtgccacaggagtgggtgcagcacttctaccaggagagcgcaccagcccagacccaggtggccctggtgcggtacgtgaacccagacaccggccgggtgctgttcgaggccaagctgcacaagctgggcttcatgaccatcgccaagaacggcgacagcccaatcaccgtgccacccaacggctacttccggttcgagagctgggtgaacccattctacaccctggccccaatgggcaccggcaacggacggaggcgggtgcag

SEQ ID NO: 14 - protein, 540 aa, synthetic construct
MKMASSDANPSDGSAANLVPEVNNEVMALEPVVGAAIAAPVAGQQNVIDPWIRNNFVQAPGGEFTVSPRNAPGEILWSAPLGPDLNPYLSHLARMYNGYAGGFEVQVILAGNAFTAGKIIFAAVPPNFPTEGLSPSQVTMFPHIIVDVRQLEPVLIPLPDVRNNFYHYNQSNDSTIKLIAMLYTPLRANNAGDDVFTVSCRVLTRPSPDFDFIFLVPPTVESRTKPFSVPVLTVEEMTNSRFPIPLEKLFTGPSSAFVVQPQNGRCTTDGVLLGTTQLSPVNICTFRGDVTHITGSRNYTMNLASQNWNNYDPTEEIPAPLGTPDFVGKIQGMLTQTTRTDGSTRGHKATVYTGSADFAPKLGRVQFETDTDHDFEANQNTKFTPVGVIQDGSTTHRNEPQQWVLPSYSGRNTHNVHLAPAVAPTFPGEQLLFFRSTMPGCSGYPNMDLDCLLPQEWVQYFYQEAAPAQSDVALLRFVNPDTGRVLFECKLHKSGYVTVAHTGQHDLVIPPNGYFRFDSWVNQFYTLAPMGNGTGRRRVV

SEQ ID NO: 15 - protein, 530 aa, synthetic construct
MMMASKDATSSVDGASGAGQLVPEVNASDPLAMDPVAGSSTAVATAGQVNPIDPWIINNFVQAPQGEFTISPNNTPGDVLFDLSLGPHLNPFLLHLSQMYNGWVGNMRVRIMLAGNAFTAGKIIVSCIPPGFGSHNLTIAQATLFPHVIADVRTLDPIEVPLEDVRNVLFHNNDRNQQTMRLVCMLYTPLRTGGGTGDSFVVAGRVMTCPSPDFNFLFLVPPTVEQKTRPFTLPNLPLSSLSNSRAPLPISSMGISPDNVQSVQFQNGRCTLDGRLVGTTPVSLSHVAKIRGTSNGTVINLTELDGTPFHPFEGPAPIGFPDLGGCDWHINMTQFGHSSQTQYDVDTTPDTFVPHLGSIQANGIGSGNYVGVLSWISPPSHPSGSQVDLWKIPNYGSSITEATHLAPSVYPPGFGEVLVFFMSKMPGPGAYNLPCLLPQEYISHLASEQAPTVGEAALLHYVDPDTGRNLGEFKAYPDGFLTCVPNGASSGPQQLPINGVFVFVSWVSRFYQLKPVGTASSARGRLGLRR

SEQ ID NO: 16 - protein, 547 aa, synthetic construct
MKMASNDAAPSNDGAANLVPEATNEVMALEPVVGASIAAPVVGQQNIIDPWIRENFVQAPQGEFTVSPRNSPGEMLLNLELGPELNPYLGHLSRMYNGYAGGMQVQVVLAGNAFTAGKIIFAAVPPHFPVENISAAQITMCPHVIVDVRQLEPVLLPLPDIRNRFFHYNQENTPRMRLVAMLYTPLRANSGEDVFTVSCRVLTRPAPDFEFTFLVPPTVESKTKPFTLPILTLGELSNSRFPAPIDMLYTDPNEAIVVQPQNGRCTLDGTLQGTTQLVPTQICSFRGTLISQTSRSADSTDSAPRVRNHPLHVQLKNLDGTPYDPTDEVPAVLGAIDFKGTVFGIASQRNTTGSSIGATRAHEVHIDTTNPRYTPKLGSVLMYSESNDFDDGQPTRFTPIGMGADDWRQWELPEYSGHLTLNMNLAPAVAPAFPGERILFFRSVVPSAGGYGSGHIDCLIPQEWVQHFYQEAAPSQSAVALIRYVNPDTGRNIFEAKLHREGFITVANSGNNPIVVPPNGYFRFEAWVNQFYTLTPMGTGQGRRRVQ

SEQ ID NO: 17 - protein, 542 aa, synthetic construct
MKMASNDAAPSTDGAAGLVPESNNEVMALEPVAGAALAAPVTGQTNIIDPWIRANFVQAPNGEFTVSPRNAPGEVLLNLELGPELNPYLAHLARMYNGYAGGMEVQVMLAGNAFTAGKLVFAAVPPHFPVENLSPQQITMFPHVIIDVRTLEPVLLPLPDVRNNFFHYNQKDDPKMRIVAMLYTPLRSNGSGDDVFTVSCRVLTRPSPDFDFTYLVPPTVESKTKPFTLPILTLGELSNSRFPVSIDQMYTSPNEIISVQCQNGRCTLDGELQGTTQLQVSGICAFKGEVTAHLHDNDHLYNVTITNLNGSPFDPSEDIPAPLGVPDFQGRVFGIISQRDKHNSPGHNEPANRGHDAVVPTYTAQYTPKLGQIQIGTWQTDDLTVNQPVKFTPVGLNDTEHFNQWVVPRYAGALNLNTNLAPSVAPVFPGERLLFFRSYIPLKGGYGNPAIDCLLPQEWVQHFYQEAAPSMSEVALVRYINPDTGRALFEAKLHRAGFMTVSSNTSAPVVVPANGYFRFDSWVNQFYSLAPMGTGNGRRRVQ

SEQ ID NO: 18 - protein, 548 aa, synthetic construct
MKMASNDATPSNDGAAGLVPEINNEAMALEPVAGAAIAAPLTGQQNIIDPWIMNNFVQAPGGEFTVSPRNSPGEVLLNLELGPEINPYLAHLARMYNGYAGGFEVQVVLAGNAFTAGKIIFAAIPPNFPIDNLSAAQITMCPHVIVDVRQLEPVNLPMPDVRNNFFHYNQGSDSRLRLVAMLYTPLRANNSGDDVFTVSCRVLTRPSPEFSFNFLVPPTVESKTKPFTLPILTISEMSNSRFPVPIDSLHTSPTENIVVQCQNGRVTLDGELMGTTQLLPSQICAFRGVLTRSTSRASDQADTATPRLFNYYWHIQLDNLNGTPYDPAEDIPGPLGTPDFRGKVFGVASQRNPDSTTRAHEAKVDTTAGRFTPKLGSLEISTESDDFDQNQPTRFTPVGIGVDNEADFQQWSLPDYSGQFTHNMNLAPAVAPNFPGEQLLFFRSQLPSSGGRSNGILDCLVPQEWVQHFYQESAPAQTQVALVRYVNPDTGRVLFEAKLHKLGFMTIAKNGDSPITVPPNGYFRFESWVNPFYTLAPMGTGNGRRRVQ

SEQ ID NO: 19 - RNA, 200 nt, synthetic construct
  source 1..200: mol_type = other RNA
  misc_difference 1..200: note = This sequence may encompass 10-200 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 20 - RNA, 200 nt, synthetic construct
  source 1..200: mol_type = other RNA
  misc_difference 1..200: note = This sequence may encompass 10-200 nucleotides
cccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccc

SEQ ID NO: 21 - RNA, 100 nt, synthetic construct
  source 1..100: mol_type = other RNA
  misc_difference 1..100: note = This sequence may encompass 10-100 nucleotides
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 22 - RNA, 80 nt, synthetic construct
  source 1..80: mol_type = other RNA
  misc_difference 1..80: note = This sequence may encompass 10-80 nucleotides
aaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 23 - RNA, 70 nt, synthetic construct
  source 1..70: mol_type = other RNA
  misc_difference 1..70: note = This sequence may encompass 50-70 nucleotides
aaaaaaaaaaaaaaaaaaaaaaaaaaaaaaaaaaaaaaaaaaaaaaaaaaaaaaaaaaaaaaaaaaaaaa

SEQ ID NO: 24 - RNA, 80 nt, synthetic construct; identical to SEQ ID NO: 22
  source 1..80: mol_type = other RNA

SEQ ID NO: 25 - RNA, 100 nt, synthetic construct
  source 1..100: mol_type = other RNA
  misc_difference 1..100: note = This sequence may encompass 10-100 nucleotides
cccccccccccccccccccccccccccccccccccccccccccccccccccccccccccccccccccccccccccccccccccccccccccccccccccc

SEQ ID NO: 26 - RNA, 70 nt, synthetic construct
  source 1..70: mol_type = other RNA
  misc_difference 1..70: note = This sequence may encompass 20-70 nucleotides
cccccccccccccccccccccccccccccccccccccccccccccccccccccccccccccccccccccc

SEQ ID NO: 27 - RNA, 60 nt, synthetic construct
  source 1..60: mol_type = other RNA
  misc_difference 1..60: note = This sequence may encompass 20-60 nucleotides
cccccccccccccccccccccccccccccccccccccccccccccccccccccccccccc

SEQ ID NO: 28 - RNA, 40 nt, synthetic construct
  source 1..40: mol_type = other RNA
  misc_difference 1..40: note = This sequence may encompass 10-40 nucleotides
cccccccccccccccccccccccccccccccccccccccc

SEQ ID NO: 29 - RNA, 400 nt, synthetic construct
  source 1..400: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 30 - RNA, 80 nt, synthetic construct
  source 1..80: mol_type = other RNA
cccccccccccccccccccccccccccccccccccccccccccccccccccccccccccccccccccccccccccccccc

SEQ ID NO: 31 - RNA, 400 nt, synthetic construct
  source 1..400: mol_type = other RNA
cccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccccc

(19) United States
(12) Patent Application Publication
(10) Pub. No.: US 2024/0376157 A1
(43) Pub. Date: Nov. 14, 2024

(54) POLYNUCLEOTIDES ENCODING NOROVIRUS VP1 ANTIGENS AND USES THEREOF

(71) Applicant: Merck Sharp & Dohme LLC, Rahway, NJ (US)

(72) Inventors: Lauren A. Austin, Lansdale, PA (US); Andrew Bett, Lansdale, PA (US); Arthur Fridman, Lake Ariel, PA (US); JiaJie Wei, Devon, PA (US); John Gaspar, Boston, MA (US); Marian E. Gindy, Haledon, NJ (US)

(73) Assignee: Merck Sharp & Dohme LLC, Rahway, NJ (US)

(21) Appl. No.: 18/655,408

(22) Filed: May 6, 2024

Related U.S. Application Data
(60) Provisional application No. 63/500,749, filed on May 8, 2023.

(51) Int. Cl. C07K 14/005; A61K 39/00; A61K 39/12; A61P 37/04; C12N 7/00

(52) U.S. Cl. CPC C07K 14/005; A61K 39/12; A61P 37/04; C12N 7/00; A61K 2039/53; A61K 2039/55555; C12N 2770/16022; C12N 2770/16023; C12N 2770/16034

(57) ABSTRACT

The present invention is directed to nucleic acids and related immunogenic polypeptides for the prevention or treatment of infectious diseases. In particular, the nucleic acids and immunogenic polypeptides provide utility for the prophylactic prevention of norovirus infections in a mammal. The invention is further directed to nucleic acid-based vaccine compositions and a kit of parts comprising the vaccine compositions, as well as methods of treatment and medical uses relating to the nucleic acids, vaccine compositions, and kit of parts.

### CROSS-REFERENCE TO RELATED APPLICATIONS

This application claims the benefit of U.S. Provisional Patent Application Ser. No. 63/500,749 filed May 8, 2023, the entire contents of which are incorporated by reference herein.

### REFERENCE TO SEQUENCE LISTING SUBMITTED ELECTRONICALLY

The contents of the electronic sequence listing (25691-WO-PCT_SL.xml; Size: 6 kilobytes; and Date of Creation: Aug. 24, 2023) are herein incorporated by reference in their entirety.

### FIELD

This disclosure relates generally to messenger ribonucleic acid (mRNA) compositions and vaccines, as well as methods of using the compositions and vaccines for the treatment of infectious diseases, such as norovirus.

### BACKGROUND

Norovirus, also referred to as “Norwalk virus,” was named after an outbreak of acute gastroenteritis in an elementary school in Norwalk, Ohio. Symptoms of norovirus infection appear within 12-48 hours of exposure and are characterized by nausea, vomiting, persistent diarrhea, lethargy, myasthenia, myalgia, headache, cough, and/or fever. Although severe illness is uncommon, and norovirus infections are generally self-limiting, norovirus remains one of the most common causes of acute gastroenteritis in people of all ages. There are approximate 700 million norovirus infections reported annually worldwide, with children under the age of five accounting for approximately 28.5% of all annual infections.

Taxonomically, noroviruses are a diverse group of single-stranded positive-sense RNA, non-enveloped viruses belonging to the family Caliciviridae. Noroviruses contain a linear, non-segmented, positive-sense RNA genome of approximately 7.5 kilobases, encoding a large polyprotein which is cleaved into six smaller non-structural proteins (NS1/2 to NS7) by the viral 3C-like protease (NS6), a major structural protein (VP1) of about 58-60 kDa and a minor capsid protein (VP2). Noroviruses can be classified genetically into at least seven different genogroups (GI, GII, GIII, GIV, GV, GVI, and GVII), which can be further divided into different genetic clusters or genotypes. Most noroviruses that infect humans belong to genogroups GI and GII. Noroviruses from genogroup II, genotype 4 (abbreviated as GII.4) account for the majority of adult outbreaks of gastroenteritis and often sweep across the globe.

Norovirus is highly contagious and transmissible, and as few as five virions may be sufficient to cause an infection. It is typically spread through contaminated water or food by the fecal-oral route of transmission. Nevertheless, airborne transmission through aerosolized virus has been documented. Hence, preventative measures such as frequent hand washing, disinfecting surfaces, and avoiding contact with infected individuals can help to reduce the risk of infection.

At present, there is no specific prophylactic or therapeutic treatment for norovirus infections. Therapy is limited to palliative care of acute infections. Hence, there is therefore a strong need for additional therapeutic measures against norovirus infections. The object of the present invention is to provide pharmaceutical compositions for the prophylactic and therapeutic treatment of norovirus infections. In particular, aspects of the present invention provide for norovirus vaccines which may be administered to a mammal for the prevention and/or treatment of a norovirus infection.

### SUMMARY

The present disclosure provides mRNA polynucleotides, pharmaceutical vaccine compositions, and kits comprising the same. The mRNA polynucleotides, vaccine compositions, and kits are useful for the prophylactic and therapeutic treatment of norovirus infections. The present disclosure additionally provides vectors and host cells comprising the mRNA polynucleotides, methods of making the pharmaceutical vaccine compositions, and methods of using the pharmaceutical vaccine compositions for the treatment of norovirus infections.

In an aspect, the present disclosure provides an mRNA polynucleotide encoding a norovirus VP1 polypeptide, wherein the mRNA polynucleotide is derived from a norovirus selected from the group consisting of: a GII.4, GI.1, GII.2, GII.3, and GII.6 norovirus, and wherein at least one uridine residue in the mRNA polynucleotide is replaced with N1-methylpseudouridine.

In an aspect, the present disclosure provides an mRNA polynucleotide encoding a norovirus VP1 polypeptide, wherein the mRNA polynucleotide is selected from the group consisting of SEQ ID NOs: 1-5 and sequences having at least 70%, 75%, 80%, 85%, 90%, 91%, 92%, 93%, 94%, 95%, 96%, 97%, 98%, or 99% identity thereto, and wherein at least one uridine residue in the mRNA polynucleotide is replaced with N1-methylpseudouridine. In certain embodiments, the norovirus VP1 polypeptide is an antigenic polypeptide.

In an aspect, the present disclosure provides an mRNA polynucleotide encoding a norovirus VP1 polypeptide, wherein the mRNA polynucleotide is selected from the group consisting of SEQ ID NOs: 1-5, and wherein at least one uridine residue in the mRNA polynucleotide is replaced with N1-methylpseudouridine.

In some embodiments, the mRNA polynucleotide is derived from a norovirus selected from the group consisting of a GII.4, GI.1, GII.2, GII.3, and GII.6 norovirus.

In some embodiments, the mRNA polynucleotide further comprises a heterologous 5′ untranslated region (UTR) and a heterologous 3′ UTR.

In some embodiments, the 5′ UTR comprises the polynucleotide sequence of SEQ ID NO: 6. In some embodiments, the 5′ UTR further comprises the 5′ cap structure N7-Methyl-G (3′Ome) ppp.

In some embodiments, the 3′ UTR comprises a poly(A) sequence and/or a poly(C) sequence. In some embodiments, the poly(A) sequence comprises from 10 to 200 adenosine nucleotides (SEQ ID NO: 19), and/or the poly(C) sequence comprises from 10 to 200 cytosine nucleotides (SEQ ID NO: 20).

In some embodiments, the poly(A) sequence comprises from 10 to 100 adenosine nucleotides (SEQ ID NO: 21). In some embodiments, the poly(A) sequence comprises from 10 to 80 adenosine nucleotides (SEQ ID NO: 22). In some embodiments, the poly(A) sequence comprises from 50 to 70 adenosine nucleotides (SEQ ID NO: 23). In certain embodiments, the poly(A) sequence comprises 80 adenine nucleotides (SEQ ID NO: 24).

In some embodiments, the poly(C) sequence comprises from 10 to 100 cytosine nucleotides (SEQ ID NO: 25). In some embodiments, the poly(C) sequence comprises from 20 to 70 cytosine nucleotides (SEQ ID NO: 26). In some embodiments, the poly(C) sequence comprises from 20 to 60 cytosine nucleotides (SEQ ID NO: 27). In some embodiments, the poly(C) sequence comprises from 10 to 40 cytosine nucleotides (SEQ ID NO: 28).

In some embodiments, the 3′ UTR comprises the polynucleotide sequence of SEQ ID NO: 7 or SEQ ID NO:8. In certain embodiments, the 3′ UTR comprises the polynucleotide sequence of SEQ ID NO:7. In certain embodiments, the 3′ UTR comprises the polynucleotide sequence of SEQ ID NO:8.

In certain embodiments, the mRNA polynucleotide comprises from the 5′ to the 3′ direction: a 5′ UTR comprising the polynucleotide sequence of SEQ ID NO:6; a polynucleotide encoding a norovirus VP1 polypeptide, wherein the polynucleotide is selected from the group consisting of SEQ ID NOs: 1-5 and sequences having at least 70%, 75%, 80%, 85%, 90%, 91%, 92%, 93%, 94%, 95%, 96%, 97%, 98%, or 99% identity thereto; and a 3′ UTR comprising the polynucleotide sequence of SEQ ID NO:7; and wherein the 5′ UTR further comprises the 5′ cap structure N7-Methyl-G (3′OMe) ppp, and all uridine residues in the polynucleotide encoding the norovirus VP1 polypeptide are replaced with N1-methylpseudouridine. In certain embodiments, the norovirus VP1 polypeptide is an antigenic polypeptide.

In certain embodiments, the mRNA polynucleotide comprises, from the 5′ to the 3′ direction: a 5′ UTR comprising the polynucleotide sequence of SEQ ID NO:6; a polynucleotide encoding a norovirus VP1 polypeptide, wherein the polynucleotide is selected from the group consisting of SEQ ID NOs: 1-5; and a 3′ UTR comprising the polynucleotide sequence of SEQ ID NO: 7; and wherein the 5′ UTR further comprises the 5′ cap structure N7-Methyl-G (3′Ome) ppp, and all uridine residues in the polynucleotide encoding the norovirus VP1 polypeptide are replaced with N1-methylpseudouridine.

In certain embodiments, the mRNA polynucleotide comprises from the 5′ to the 3′ direction: a 5′ UTR comprising the polynucleotide sequence of SEQ ID NO:6; a polynucleotide encoding a norovirus VP1 polypeptide, wherein the polynucleotide is selected from the group consisting of SEQ ID NOs: 1-5 and sequences having at least 70%, 75%, 80%, 85%, 90%, 91%, 92%, 93%, 94%, 95%, 96%, 97%, 98%, or 99% identity thereto; and a 3′ UTR comprising the polynucleotide sequence of SEQ ID NO:8; and wherein the 5′ UTR further comprises the 5′ cap structure N7-Methyl-G (3′OMe) ppp, and all uridine residues in the polynucleotide encoding the norovirus VP1 polypeptide are replaced with N1-methylpseudouridine. In certain embodiments, the norovirus VP1 polypeptide is an antigenic polypeptide.

In certain embodiments, the mRNA polynucleotide comprises, from the 5′ to the 3′ direction: a 5′ UTR comprising the polynucleotide sequence of SEQ ID NO:6; a polynucleotide encoding a norovirus VP1 polypeptide, wherein the polynucleotide is selected from the group consisting of SEQ ID NOs: 1-5; and a 3′ UTR comprising the polynucleotide sequence of SEQ ID NO: 8; and wherein the 5′ UTR further comprises the 5′ cap structure N7-Methyl-G (3′OMe) ppp, and all uridine residues in the polynucleotide encoding the norovirus VP1 polypeptide are replaced with N1-methylpseudouridine.

In some embodiments, the mRNA polynucleotide comprises at least one histone stem-loop structure.

In some embodiments, at least 50% of the uridine residues in the mRNA polynucleotide are replaced with N1-methylpseudouridine. In some embodiments, at least 75% of the uridine residues in the mRNA polynucleotide are replaced with N1-methylpseudouridine. In some embodiments, at least 85% of the uridine residues in the mRNA polynucleotide are replaced with N1-methylpseudouridine. In some embodiments, at least 90% of the uridine residues in the mRNA polynucleotide are replaced with N1-methylpseudouridine. In some embodiments, at least 95% of the uridine residues in the mRNA polynucleotide are replaced with N1-methylpseudouridine. In some embodiments, at least 96% of the uridine residues in the mRNA polynucleotide are replaced with N1-methylpseudouridine. In some embodiments, at least 97% of the uridine residues in the mRNA polynucleotide are replaced with N1-methylpseudouridine. In some embodiments, at least 98% of the uridine residues in the mRNA polynucleotide are replaced with N1-methylpseudouridine. In some embodiments, at least 99% of the uridine residues in the mRNA polynucleotide are replaced with N1-methylpseudouridine. In certain embodiments, 100% of the uridine residues in the mRNA polynucleotide are replaced with N1-methylpseudouridine.

In another aspect, the present disclosure provides a combination of one or more mRNA polynucleotides as described herein. In specific embodiments, the one or more mRNA polynucleotides is 1-10, 1-9, 1-8, 1-7, 1-6, 1-5, 1-4, 1-3, or 1-2 mRNA polynucleotides as described herein. In specific embodiments, the one or more mRNA polynucleotides is at least one, at least two, at least three, at least four, at least five, or five mRNA polynucleotides as described herein.

In another aspect, the present disclosure provides a composition comprising at least one mRNA polynucleotide encoding a norovirus VP1 polypeptide as described herein and a pharmaceutically acceptable carrier.

In another aspect, the present disclosure provides a composition comprising at least two mRNA polynucleotides encoding norovirus VP1 polypeptides as described herein and a pharmaceutically acceptable carrier.

In another aspect, the present disclosure provides a composition comprising at least three mRNA polynucleotides encoding norovirus VP1 polypeptides as described herein and a pharmaceutically acceptable carrier.

In another aspect, the present disclosure provides a composition comprising at least four mRNA polynucleotides encoding norovirus VP1 polypeptides as described herein and a pharmaceutically acceptable carrier.

In another aspect, the present disclosure provides a composition comprising at least five mRNA polynucleotides encoding norovirus VP1 polypeptides as described herein and a pharmaceutically acceptable carrier. In some embodiments, the at least five mRNA polynucleotides are derived from GII.4, GI.1, GII.2, GII.3, and GII.6 norovirus. In some embodiments, the composition comprises five mRNA polynucleotides, each derived from one of GII.4, GI.1, GII.2, GII.3, and GII.6 norovirus, and a pharmaceutically acceptable carrier.

In another aspect, the present disclosure provides a composition comprising five mRNA polynucleotides encoding norovirus VP1 polypeptides and a pharmaceutically acceptable carrier, wherein each of the five mRNA polynucleotides comprises, from the 5′ to the 3′ direction: a 5′ UTR comprising the polynucleotide sequence of SEQ ID NO:6; a polynucleotide encoding a norovirus VP1 polypeptide, wherein the polynucleotide is selected from the group consisting of SEQ ID NOs: 1-5 and sequences having at least 70%, 75%, 80%, 85%, 90%, 91%, 92%, 93%, 94%, 95%, 96%, 97%, 98%, or 99% identity thereto; and a 3′ UTR comprising the polynucleotide sequence of SEQ ID NO:7; and wherein the 5′ UTR further comprises the 5′ cap structure N7-Methyl-G (3′OMe) ppp, and wherein the composition comprises each of SEQ ID NOs: 1-5, or sequences having at least 70%, 75%, 80%, 85%, 90%, 91%, 92%, 93%, 94%, 95%, 96%, 97%, 98%, or 99% identity thereto, wherein in each of SEQ ID NOs: 1-5, or sequences having at least 70%, 75%, 80%, 85%, 90%, 91%, 92%, 93%, 94%, 95%, 96%, 97%, 98%, or 99% identity thereto, all uridine residues in the five mRNA polynucleotide are replaced with N1-methylpseudouridine. In certain embodiments, the norovirus VP1 polypeptide is an antigenic polypeptide.

In another aspect, the present disclosure provides a composition comprising five mRNA polynucleotides encoding norovirus VP1 polypeptides and a pharmaceutically acceptable carrier, wherein each of the five mRNA polynucleotides comprises, from the 5′ to the 3′ direction: a 5′ UTR comprising the polynucleotide sequence of SEQ ID NO:6; a polynucleotide encoding a norovirus VP1 polypeptide, wherein the polynucleotide is selected from the group consisting of SEQ ID NOs: 1-5; and a 3′ UTR comprising the polynucleotide sequence of SEQ ID NO:7; and wherein the 5′ UTR further comprises the 5′ cap structure N7-Methyl-G (3′OMc) ppp, and wherein the composition comprises each of SEQ ID NOs: 1-5, wherein in each of SEQ ID NOs: 1-5 all uridine residues in the five mRNA polynucleotide are replaced with N1-methylpseudouridine.

In another aspect, the present disclosure provides a composition comprising five mRNA polynucleotides encoding norovirus VP1 polypeptides and a pharmaceutically acceptable carrier, wherein each of the five mRNA polynucleotides comprises, from the 5′ to the 3′ direction: a 5′ UTR comprising the polynucleotide sequence of SEQ ID NO:6; a polynucleotide encoding a norovirus VP1 polypeptide, wherein the polynucleotide is selected from the group consisting of SEQ ID NOs: 1-5 and sequences having at least 70%, 75%, 80%, 85%, 90%, 91%, 92%, 93%, 94%, 95%, 96%, 97%, 98%, or 99% identity thereto; and a 3′ UTR comprising the polynucleotide sequence of SEQ ID NO:8; and wherein the 5′ UTR further comprises the 5′ cap structure N7-Methyl-G (3′OMe) ppp, and wherein the composition comprises each of SEQ ID NOs: 1-5, o sequences having at least 70%, 75%, 80%, 85%, 90%, 91%, 92%, 93%, 94%, 95%, 96%, 97%, 98%, or 99% identity thereto, wherein in each of SEQ ID NOs: 1-5, or sequences having at least 70%, 75%, 80%, 85%, 90%, 91%, 92%, 93%, 94%, 95%, 96%, 97%, 98%, or 99% identity thereto, all uridine residues in the five mRNA polynucleotides are replaced with N1-methylpseudouridine. In certain embodiments, the norovirus VP1 polypeptide is an antigenic polypeptide.

In another aspect, the present disclosure provides a composition comprising five mRNA polynucleotides encoding norovirus VP1 polypeptides and a pharmaceutically acceptable carrier, wherein each of the five mRNA polynucleotides comprises, from the 5′ to the 3′ direction: a 5′ UTR comprising the polynucleotide sequence of SEQ ID NO:6; a polynucleotide encoding a norovirus VP1 polypeptide, wherein the polynucleotide is selected from the group consisting of SEQ ID NOs: 1-5; and a 3′ UTR comprising the polynucleotide sequence of SEQ ID NO:8; and wherein the 5′ UTR further comprises the 5′ cap structure N7-Methyl-G (3′OMe) ppp, and wherein the composition comprises each of SEQ ID NOs: 1-5, wherein in each of SEQ ID NOs: 1-5 all uridine residues in the five mRNA polynucleotides are replaced with N1-methylpseudouridine.

In some embodiments, the composition further comprises one or more additional mRNA polynucleotides derived from a norovirus selected from the group consisting of a genogroup I norovirus, a genogroup II norovirus, a genogroup III norovirus, a genogroup IV norovirus, and a genogroup V norovirus. In some embodiments, the norovirus VP1 polypeptide of the composition is derived from genogroup I norovirus or genogroup II norovirus. In some embodiments, the norovirus VP1 polypeptide of the composition is derived from a genogroup selected from the group consisting of: a GII.4, GI.1, GII.2, GII.3, and GII.6 norovirus. In some embodiments, the norovirus VP1 polypeptide of the composition is selected from the group consisting of SEQ ID NOs: 14-18 and sequences having at least 70% identity thereto.

In another aspect, the present disclosure provides a composition comprising at least one mRNA polynucleotide encoding a norovirus VP1 polypeptide and a pharmaceutically acceptable carrier. In some embodiments, the composition further comprises at least one, at least two, at least three, at least four or at least five mRNA polynucleotide encoding a norovirus VP1 polypeptide.

In some embodiments, each mRNA polynucleotide is encapsulated separately in a lipid nanoparticle (LNP) comprising one or more cationic or polycationic compounds.

In some embodiments, each mRNA polynucleotide is encapsulated together in a LNP comprising one or more cationic or polycationic compounds.

In some embodiments, each mRNA polynucleotide encapsulated in the LNP is present in an equal amount.

In some embodiments, each mRNA polynucleotide encapsulated in the LNP is not present in an equal amount.

In some embodiments, the LNP comprises one or more of a cationic lipid, a sterol, a phospholipid, and a polyethyleneglycol-lipid. In other embodiments, the LNP comprises a cationic lipid, a sterol, a phospholipid, and a polyethyleneglycol-lipid.

In some embodiments, the LNP comprises 34-59 mole % of a cationic lipid, 30-48 mole % of a sterol, 10-24 mole % of a phospholipid, and 1-2 mole % of a polyethyleneglycol-lipid.

In some embodiments, the cationic lipid is selected from the group consisting of:

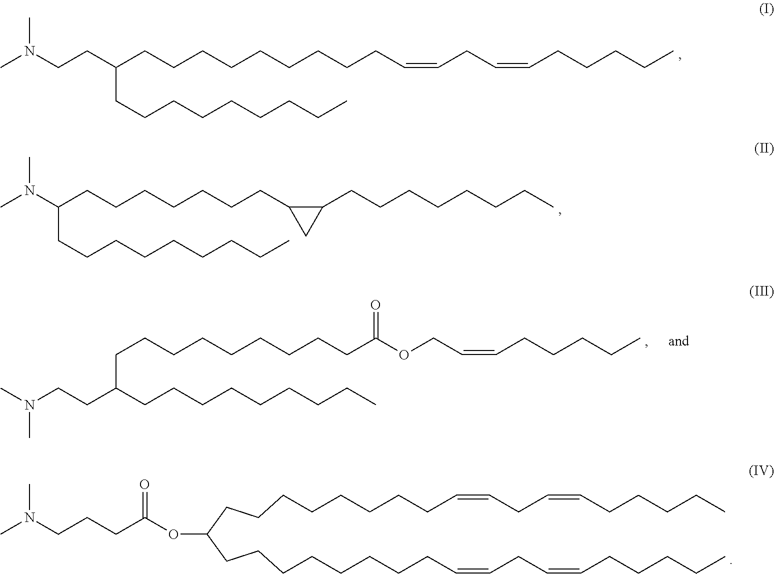

In some embodiments, the sterol is:

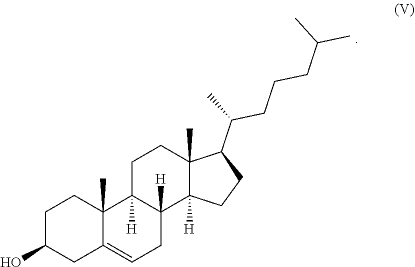

In some embodiments, wherein the phospholipid is selected from the group consisting of:

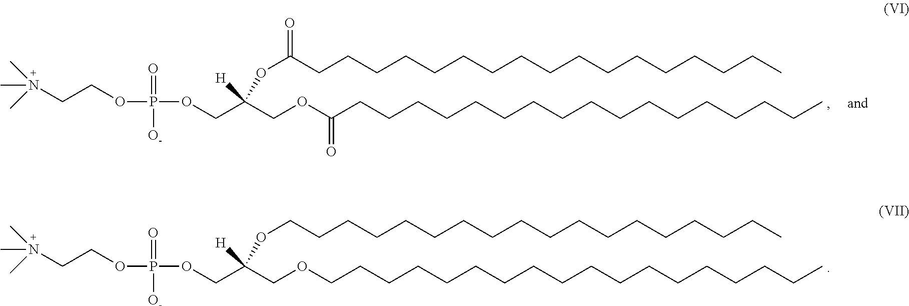

In some embodiments, the polyethyleneglycol-lipid is selected from the group consisting of:

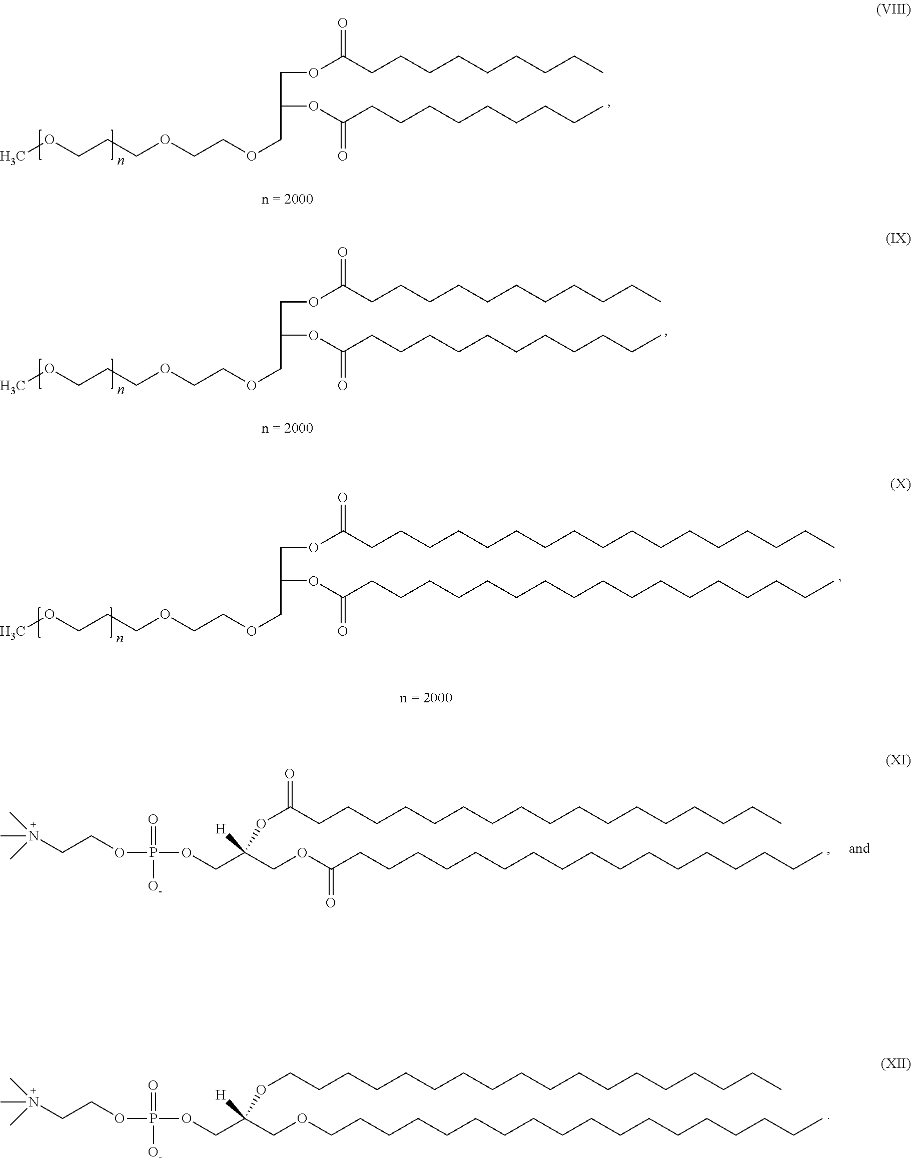

In some embodiments, the cationic lipid is (13Z, 16Z)—N,N-dimethyl-3-nonyldocosa-13,16-dien-1-amine.

In some embodiments, the sterol is cholesterol.

In some embodiments, the phospholipid is distearoylphosphatidylcholine (DSPC).

In some embodiments, the polyethyleneglycol-lipid is dimyristoyl glycerol-polyethyleneglycol (DMG-PEG).

In some embodiments, the LNP comprises 49-59 mole % (13Z, 16Z)—N,N-dimethyl-3-nonyldocosa-13,16-dien-1-amine. In other embodiments, the LNP comprises about 58 mole % (13Z, 16Z)—N,N-dimethyl-3-nonyldocosa-13,16-dien-1-amine. In certain embodiments, the LNP comprises about 49 mole % (13Z, 16Z)—N,N-dimethyl-3-nonyldocosa-13,16-dien-1-amine.

In another aspect, the present disclosure provides a vaccine comprising an mRNA polynucleotide, combination, or composition as described herein.

In some embodiments, the vaccine further comprises a pharmaceutically acceptable carrier. In some embodiments, the vaccine further comprises an adjuvant.

In some embodiments, the vaccine is monovalent, bivalent, trivalent, quadrivalent, or pentavalent.

In some embodiments, the mRNA polynucleotide, the combination, the composition, and/or the vaccine as described herein are immunogenic in a mammalian species.

In another aspect, the present disclosure provides a kit or kit of parts comprising an mRNA polynucleotide, combination, composition, or vaccine as described herein, wherein the kit or kit of parts optionally further comprises a liquid vehicle for solubilizing, and/or technical instructions providing information on administration and dosage of the components.

In another aspect, the present disclosure provides an mRNA polynucleotide, combination, composition, vaccine, or kit or kit of parts as described herein, for use as a medicament.

In another aspect, the present disclosure provides an mRNA polynucleotide, combination, composition, vaccine, or kit or kit of parts as described herein, for use in the manufacture of a medicament for the treatment or prophylaxis of an infection with norovirus or a disorder related to an infection with norovirus.

In another aspect, the present disclosure provides an mRNA polynucleotide, combination, composition, vaccine, or kit or kit of parts as described herein, for use in the treatment or prophylaxis of an infection with norovirus or a disorder related to an infection with norovirus.

In another aspect, the present disclosure provides an mRNA polynucleotide, combination, composition, vaccine, or kit or kit of parts as described herein, wherein an effective amount of the polynucleotide, the combination, the composition, the vaccine or the active component of the kit or kit of parts is administered by injection.

In another aspect, the present disclosure provides a method of treating or preventing a disorder, wherein the method comprises administering to a subject in need thereof an effective amount of an mRNA polynucleotide, combination, composition, vaccine, or active ingredient of the kit or kit of parts as described herein. In some embodiments, the disorder is an infection with norovirus or a disorder related to an infection with norovirus.

In another aspect, the present disclosure provides a vector comprising one or more nucleic acids encoding an mRNA polynucleotide as described herein.

In another aspect, the present disclosure provides a host cell comprising an mRNA polynucleotide, combination, or vector as described herein.

In another aspect, the present disclosure provides a polypeptide encoded by an mRNA polynucleotide as described herein.

In another aspect, the present disclosure provides a method of making a virus-like particle (VLP) comprising one or more norovirus VP1 polypeptide, wherein the method comprises transcribing an mRNA polynucleotide, combination, or vector as described herein in a recombinant nucleic acid expression system under conditions suitable for assembly of the expressed one or more norovirus VP1 polypeptide into a VLP.

In another aspect, the present disclosure provides a VLP comprising one or more norovirus VP1 polypeptide produced according to the method described herein.

The summary of the technology described above is non-limiting and other features and advantages of the technology will be apparent from the following detailed description, and from the claims.

### BRIEF DESCRIPTION OF THE DRAWINGS

FIG. 1 shows a 2% agarose E-gel of purified mRNA for the five norovirus VP1 encoded sequences.

FIGS. 2A-2E show a plot depicting the interpolated endpoint titers detected in sera of mice vaccinated with the test vaccines. FIG. 2A shows titer for GI.1; FIG. 2B shows titer for GII.2; FIG. 2C shows titer for GII.3; FIG. 2D shows titer for GII.4; and FIG. 2E shows titer for GII.6.

FIGS. 3A-3E show a plot depicting the histo-blood group antigen (HBGA) blocking titers detected in sera of mice vaccinated with the test vaccines. FIG. 3A shows titer for GI.1;

FIG. 3B shows titer for GII.2; FIG. 3C shows titer for GII.3; FIG. 3D shows titer for GII.4; and FIG. 3E shows titer for GII.6.

FIGS. 4A-4E show a plot depicting the interpolated endpoint titers detected in sera of NHPs vaccinated with the test vaccines. FIG. 4A shows titer for GI.1; FIG. 4B shows titer for GII.2; FIG. 4C shows titer for GII.3; FIG. 4D shows titer for GII.4; and FIG. 4E shows titer for GII.6.

FIG. 5A-5E show a plot depicting the HBGA blocking titers detected in sera of NHPs vaccinated with the test vaccines. FIG. 5A shows titer for GI.1; FIG. 5B shows titer for GII.2;

FIG. 5C shows titer for GII.3; FIG. 5D shows titer for GII.4; and FIG. 5E shows titer for GII.6.

### DETAILED DESCRIPTION

The present disclosure provides mRNA polynucleotides, pharmaceutical vaccine compositions, and kits comprising the same. The mRNA polynucleotides, vaccine compositions, and kits are useful for the prophylactic and therapeutic treatment of norovirus infections. The present disclosure additionally provides vectors and host cells comprising the mRNA polynucleotides, methods of making the pharmaceutical vaccine compositions, and methods of using the pharmaceutical vaccine compositions for the treatment of norovirus infections.

### Definitions

Listed herein are definitions of various terms used herein. These definitions apply to the terms as they are used throughout this specification and claims, unless otherwise limited in specific instances, either individually or as part of a larger group.

Unless defined otherwise, all technical and scientific terms used herein have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” in quantitative terms refers to plus or minus 10% of the value it modifies (rounded up to the nearest whole number if the value is not sub-dividable, such as a number of molecules or nucleotides).

All ranges disclosed herein are inclusive of the recited endpoint and independently combinable (for example, the range of “from 50 mg to 500 mg” is inclusive of the endpoints, 50 mg and 500 mg, and all the intermediate values). The endpoints of the ranges and any values disclosed herein are not limited to the precise range or value; they are sufficiently imprecise to include values approximating these ranges and/or values.

As used herein, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated components, which allows the presence of only the named components or compounds, along with any acceptable carriers or fluids, and excludes other components or compounds.

### Nucleic Acids/Polynucleotides

Norovirus vaccines, as provided herein, comprise at least one (one or more) ribonucleic acid (RNA) polynucleotide having an open reading frame encoding at least one norovirus antigenic polypeptide. The term “nucleic acid,” in its broadest sense, includes any compound and/or substance that comprises a polymer of nucleotides. These polymers are referred to as “polynucleotides.” Nucleic acids (also referred to as polynucleotides) may be or may include, for example, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization), ethylene nucleic acids (ENA), cyclohexenyl nucleic acids (CeNA), or chimeras or combinations thereof.

In some embodiments, polynucleotides of the present disclosure function as messenger RNA (mRNA). “Messenger RNA” (mRNA) refers to any polynucleotide that encodes a (at least one) polypeptide (a naturally-occurring, non-naturally-occurring, or modified polymer of amino acids) and can be translated to produce the encoded polypeptide in vitro, in vivo, in situ or ex vivo. The skilled artisan will appreciate that, except where otherwise noted, polynucleotide sequences set forth in the instant application will recite “T”'s in a representative DNA sequence but where the sequence represents RNA, the “T”'s would be substituted for “U”'s.

The basic components of an mRNA molecule typically include at least one coding region, a 5′ untranslated region (UTR), a 3′ UTR, a 5′ cap, and a poly(A) tail. Polynucleotides of the present disclosure may function as mRNA but can be distinguished from wild-type mRNA in their functional and/or structural design features which serve to overcome existing problems of effective polypeptide expression using nucleic-acid based therapeutics.

In some embodiments, an RNA polynucleotide of a norovirus vaccine encodes 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 antigenic polypeptides. In some embodiments, an RNA polynucleotide of a norovirus vaccine encodes an antigenic polypeptide derived from a single norovirus genotype, i.e., the vaccine is “monovalent.” In some embodiments, the RNA polynucleotides of a norovirus vaccine encodes antigenic polypeptides derived from two distinct norovirus genotypes, i.e., the vaccine is “bivalent.” In some embodiments, the RNA polynucleotides of a norovirus vaccine encodes antigenic polypeptides derived from three distinct norovirus genotypes, i.e., the vaccine is “trivalent.” In some embodiments, the RNA polynucleotide of a norovirus vaccine encodes antigenic polypeptides derived from four distinct norovirus genotypes, i.e., the vaccine is “quadrivalent.” In certain embodiments, the RNA polynucleotides of the norovirus vaccine encodes antigenic polypeptides derived from five distinct norovirus genotypes, i.e., the vaccine is “pentavalent.”

In some embodiments, polynucleotides of the present disclosure are codon optimized. Codon optimization methods are known in the art and may be used as provided herein. Codon optimization, in some embodiments, may be used to match codon frequencies in target and host organisms to ensure proper folding; bias GC content to increase mRNA stability or reduce secondary structures; minimize tandem repeat codons or base runs that may impair gene construction or expression; customize transcriptional and translational control regions; insert or remove protein trafficking sequences; remove/add post translation modification sites in encoded protein (e.g., glycosylation sites); add, remove or shuffle protein domains; insert or restriction sites; modify ribosome binding sites and mRNA degradation sites; adjust translational rates to allow the various domains of the protein to fold properly; or to reduce or eliminate problem secondary structures within the polynucleotide. Codon optimization tools, algorithms and services are known in the art-non-limiting examples include services from GeneArt (Life Technologies), DNA2.0 (Menlo Park CA), and/or proprietary methods. In some embodiments, the open reading frame (ORF) sequence is optimized using optimization algorithms.

In some embodiments, a codon optimized sequence shares less than 95% sequence identity, less than 90% sequence identity, less than 85% sequence identity, less than 80% sequence identity, or less than 75% sequence identity to a naturally-occurring or wild-type sequence. In certain embodiments of the present invention, the codon optimized polynucleotide sequence is any one of SEQ ID NOs: 1-5 or 9-13, or a sequence having at least 70%, 75%, 80%, 85%, 90%, 91%, 92%, 93%, 94%, 95%, 96%, 97%, 98%, or 99% identity thereto. In certain embodiments of the present invention, the codon optimized polynucleotide sequence is any one of SEQ ID NOs: 1-5 or 9-13.

In addition to a coding region, a mature mRNA comprises a 5′ untranslated region (UTR) and a 3′ UTR, which play important roles in regulating gene expression. A “5′ UTR” refers to a region of an mRNA that is directly upstream (i.e., 5′) from the start codon (i.e., the first codon of an mRNA transcript translated by a ribosome) that does not encode a polypeptide. A “3′ UTR” refers to a region of an mRNA that is directly downstream (i.e., 3′) from the stop codon (i.e., the codon of an mRNA transcript that signals a termination of translation) that does not encode a polypeptide. In some embodiments of the present invention, the 5′ UTR is SEQ ID NO: 6. In some embodiments, the 3′ UTR is SEQ ID NO: 7 or SEQ ID NO:8.

The 5′ UTR may further comprise a 5′ cap sequence. 5′ capping of polynucleotides may be completed concomitantly during an in vitro-transcription reaction using the following chemical RNA cap analogs to generate the 5′ guanosine cap structure according to manufacturer protocols: 3′-O-Me-m7G (5′) ppp (5′) G [the ARCA cap];G (5′) ppp (5′) A; G (5′) ppp (5′) G; m7G (5′) ppp (5′) A; m7G (5′) ppp (5′) G (New England BioLabs, Ipswich, MA). 5′ capping of modified RNA may be completed post-transcriptionally using a Vaccinia Virus Capping Enzyme to generate the “Cap 0” structure: m7G (5′) ppp (5′) G (New England BioLabs, Ipswich, MA). Cap 1 structure may be generated using both Vaccinia Virus Capping Enzyme and a 2′-O methyl-transferase to generate: m7G (5′) ppp (5′) G-2′-O-methyl. Cap 2 structure may be generated from the Cap 1 structure followed by the 2′-O-methylation of the 5′-antepenultimate nucleotide using a 2′-O methyl-transferase. Cap 3 structure may be generated from the Cap 2 structure followed by the 2′-O-methylation of the 5′-preantepenultimate nucleotide using a 2′-O methyl-transferase. Enzymes are preferably derived from a recombinant source. In certain embodiments of the present invention, the 5′ cap structure is N7-Methyl-G (3′OMe) ppp.

The 3′ UTR may further comprise a poly(A) sequence or a poly(C) sequence, which are typically located at the 3′ end of an mRNA. A poly(A) sequence, also called poly(A) tail or 3′ poly(A) tail, is typically understood to be a sequence of adenosine nucleotides, e.g., of up to about 400 adenosine nucleotides (SEQ ID NO: 29), e.g., from about 10 to about 400, preferably from about 10 to about 200, more preferably from about 20 to about 80, even more preferably from about 40 to about 80, most preferably from about 60 to about 80 adenosine nucleotides. In certain embodiments of the present invention, the poly(A) sequence comprises 80 adenine nucleotides (SEQ ID NO: 24). A poly(C) sequence, also called poly(C) tail or 3′ poly(C) tail, is typically understood to be a sequence of cytidine e nucleotides, e.g., of up to about 400 cytidine nucleotides (SEQ ID NO: 31), e.g., from about 10 to about 400, preferably from about 10 to about 200, more preferably from about 20 to about 80, even more preferably from about 40 to about 80, most preferably from about 60 to about 80 cytidine nucleotides. In certain embodiments of the present invention, the poly(C) sequence comprises 80 cytidine nucleotides (SEQ ID NO: 30).

In the context of the present invention, a poly(A) sequence may be located within an mRNA or any other nucleic acid molecule, such as, e.g., in a vector, for example, in a vector serving as template for the generation of an RNA, preferably an mRNA, e.g., by transcription of the vector. Moreover, poly(A) sequences, or poly(A) tails may be generated in vitro by enzymatic polyadenylation of the RNA, e.g., using Poly(A) polymerases (PAP) derived from E. coli or yeast. In addition, polyadenylation of RNA can be achieved by using immobilized PAP enzymes e.g., in a polyadenylation reactor. See International Patent Application Publication WO 2016/174271.

### Chemical Modifications

RNA vaccines of the present disclosure comprise at least one ribonucleic acid (RNA) polynucleotide having an open reading frame encoding at least one norovirus antigenic polypeptide that comprises at least one chemical modification.

The terms “chemical modification” and “chemically modified” refer to modification with respect to adenosine (A), guanosine (G), uridine (U), thymidine (T), or cytidine (C) ribonucleosides or deoxyribonucleosides in at least one of their position, pattern, percent, or population. Generally, these terms do not refer to the ribonucleotide modifications in naturally occurring 5′ terminal mRNA cap moieties. With respect to a polypeptide, the term “modification” refers to a modification relative to the canonical set 20 amino acids. Polypeptides, as provided herein, are also considered “modified” of they contain amino acid substitutions, insertions or a combination of substitutions and insertions.

Polynucleotides (e.g., RNA polynucleotides, such as mRNA polynucleotides), in some embodiments, comprise various (more than one) different modifications. In some embodiments, a particular region of a polynucleotide contains one, two, or more (optionally different) nucleoside or nucleotide modifications. In some embodiments, a modified RNA polynucleotide (e.g., a modified mRNA polynucleotide), introduced to a cell or organism, exhibits reduced degradation in the cell or organism, respectively, relative to an unmodified polynucleotide. In some embodiments, a modified RNA polynucleotide (e.g., a modified mRNA polynucleotide), introduced into a cell or organism, may exhibit reduced immunogenicity in the cell or organism, respectively (e.g., a reduced innate response).

Modifications of polynucleotides include, without limitation, those described herein. Polynucleotides (e.g., RNA polynucleotides, such as mRNA polynucleotides) may comprise modifications that are naturally-occurring, non-naturally-occurring or the polynucleotide may comprise a combination of naturally-occurring and non-naturally-occurring modifications. Polynucleotides may include any useful modification, for example, of a sugar, a nucleobase, or an internucleoside linkage (e.g., to a linking phosphate, to a phosphodiester linkage or to the phosphodiester backbone).

Polynucleotides (e.g., RNA polynucleotides, such as mRNA polynucleotides), in some embodiments, comprise non-natural modified nucleotides that are introduced during synthesis or post-synthesis of the polynucleotides to achieve desired functions or properties. The modifications may be present on an internucleotide linkages, purine or pyrimidine bases, or sugars. The modification may be introduced with chemical synthesis or with a polymerase enzyme at the terminal of a chain or anywhere else in the chain. Any of the regions of a polynucleotide may be chemically modified.

The present disclosure provides for modified nucleosides and nucleotides of a polynucleotide (e.g., RNA polynucleotides, such as mRNA polynucleotides). A “nucleoside” refers to a compound containing a sugar molecule (e.g., a pentose or ribose) or a derivative thereof in combination with an organic base (e.g., a purine or pyrimidine) or a derivative thereof (also referred to herein as “nucleobase”). A nucleotide” refers to a nucleoside, including a phosphate group. Modified nucleotides may by synthesized by any useful method, such as, for example, chemically, enzymatically, or recombinantly, to include one or more modified or non-natural nucleosides. Polynucleotides may comprise a region or regions of linked nucleosides. Such regions may have variable backbone linkages. The linkages may be standard phosphodiester linkages, in which case the polynucleotides would comprise regions of nucleotides.

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine, or uracil. Any combination of base/sugar or linker may be incorporated into polynucleotides of the present disclosure.

Modifications of polynucleotides (e.g., RNA polynucleotides, such as mRNA polynucleotides) that are useful in the vaccines of the present disclosure include, but are not limited to the following: 2-methylthio-N6-(cis-hydroxyisopentenyl) adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl) adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6,N6 (dimethyl) adenine; N6-cis-hydroxy-isopentenyl-adenosine; α-thio-adenosine; 2 (amino) adenine; 2 (aminopropyl) adenine; 2 (methylthio) N6 (isopentenyl) adenine; 2-(alkyl) adenine; 2-(aminoalkyl) adenine; 2-(aminopropyl) adenine; 2-(halo) adenine; 2-(halo) adenine; 2-(propyl) adenine; 2′-Amino-2′-deoxy-ATP; 2′-Azido-2′-deoxy-ATP; 2′-Deoxy-2′-α-aminoadenosine TP; 2′-Deoxy-2′-α-azidoadenosine TP; 6 (alkyl) adenine; 6 (methyl) adenine; 6-(alkyl) adenine; 6-(methyl) adenine; 7 (deaza) adenine; 8 (alkenyl) adenine; 8 (alkynyl) adenine; 8 (amino) adenine; 8 (thioalkyl) adenine; 8-(alkenyl) adenine; 8-(alkyl) adenine; 8-(alkynyl) adenine; 8-(amino) adenine; 8-(halo) adenine; 8-(hydroxyl) adenine; 8-(thioalkyl) adenine; 8-(thiol) adenine; 8-azido-adenosine; aza adenine; deaza adenine; N6 (methyl) adenine; N6-(isopentyl) adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; α-thio-cytidine; 2-(thio) cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza) cytosine; 3 (methyl) cytosine; 3-(alkyl) cytosine; 3-(deaza) 5 (aza) cytosine; 3-(methyl) cytidine; 4,2′-O-dimethylcytidine; 5 (halo) cytosine; 5 (methyl) cytosine; 5 (propynyl) cytosine; 5 (trifluoromethyl) cytosine; 5-(alkyl) cytosine; 5-(alkynyl) cytosine; 5-(halo) cytosine; 5-(propynyl) cytosine; 5-(trifluoromethyl) cytosine; 5-bromo-cytidine; 5-iodo-cytidine; 5-propynyl cytosine; 6-(azo) cytosine; 6-aza-cytidine; aza cytosine; deaza cytosine; N4 (acetyl) cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl) cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl) cytidine TP; 3′-Ethynylcytidine TP; 4′-Azidocytidine TP; 4′-Carbocyclic cytidine TP; 4′-Ethynylcytidine TP; 5-(1-Propynyl) ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archacosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; α-thio-guanosinc; 2 (propyl) guanine; 2-(alkyl) guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl) guaninc; 6-(alkyl) guaninc; 6-(methyl) guaninc; 6-methyl-guanosine; 7 (alkyl) guanine; 7 (deaza) guanine; 7 (methyl) guanine; 7-(alkyl) guanine; 7-(deaza) guanine; 7-(methyl) guaninc; 8 (alkyl) guanine; 8 (alkynyl) guanine; 8 (halo) guanine; 8 (thioalkyl) guanine; 8-(alkenyl) guanine; 8-(alkyl) guaninc; 8-(alkynyl) guanine; 8-(amino) guanine; 8-(halo) guanine; 8-(hydroxyl) guanine; 8-(thioalkyl) guanine; 8-(thiol) guanine; aza guanine; deaza guanine; N (methyl) guanine; N-(methyl) guaninc; 1-methyl-6-thio-guanosinc; 6-methoxy-guanosine; 6-thio-7-deaza-8-aza-guanosine; 6-thio-7-deaza-guanosine; 6-thio-7-methyl-guanosine; 7-deaza-8-aza-guanosinc; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosinc TP; 2′-Deoxy-2′-a-thiomethoxyguanosine TP; 2′-Deoxy-2′-b-aminoguanosinc TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl) uridine; 1-methyl-3-(3-amino-5-carboxypropyl) pseudouridine; 1-methylpseduouridine; 1-methyl-pseudouridine; 2′-O-methyluridine; 2′-O-methylpseudouridine; 2′-O-methyluridine; 2-thio-2′-O-methyluridine; 3-(3-amino-3-carboxypropyl) uridine; 3,2′-O-dimethyluridine; 3-Methyl-pseudo-Uridine TP; 4-thiouridine; 5-(carboxyhydroxymethyl) uridine; 5-(carboxyhydroxymethyl) uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudouridine; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(iso-Pentenylaminomethyl)-2′-O-methyluridine TP; 5-(iso-Pentenylaminomethyl) uridine TP; 5-propynyl uracil; α-thio-uridine; 1 (aminoalkylamino-carbonylethylenyl)-2 (thio)-pseudouracil; 1 (aminoalkylaminocarbonylethylenyl)-2,4-(dithio) pseudouracil; 1 (aminoalkylaminocarbonylethylenyl)-4 (thio) pseudouracil; 1 (aminoalkylaminocarbonylethylenyl)-pseudouracil; 1 (aminocarbonylethylenyl)-2 (thio)-pseudouracil; 1 (aminocarbonylethylenyl)-2,4-(dithio) pseudouracil; 1 (aminocarbonylethylenyl)-4 (thio) pseudouracil; 1 (aminocarbonylethylenyl)-pseudouracil; 1 substituted 2 (thio)-pseudouracil; 1 substituted 2,4-(dithio) pseudouracil; 1 substituted 4 (thio) pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl) pseudo-UTP; 1-Methyl-pseudo-UTP; 2 (thio) pseudouracil; 2′ deoxy uridine; 2′ fluorouridine; 2-(thio) uracil; 2,4-(dithio) psuedouracil; 2′ methyl, 2′amino, 2′azido, 2′fluro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl) uracil; 4 (thio) pseudouracil; 4-(thio) pseudouracil; 4-(thio) uracil; 4-thiouracil; 5 (1,3-diazole-1-alkyl) uracil; 5 (2-aminopropyl) uracil; 5 (aminoalkyl) uracil; 5 (dimethylaminoalkyl) uracil; 5 (guanidiniumalkyl) uracil; 5 (methoxycarbonylmethyl)-2-(thio) uracil; 5 (methoxycarbonyl-methyl) uracil; 5 (methyl) 2 (thio) uracil; 5 (methyl) 2,4 (dithio) uracil; 5 (methyl) 4 (thio) uracil; 5 (methylaminomethyl)-2 (thio) uracil; 5 (methylaminomethyl)-2,4 (dithio) uracil; 5 (methylaminomethyl)-4 (thio) uracil; 5 (propynyl) uracil; 5 (trifluoromethyl) uracil; 5-(2-aminopropyl) uracil; 5-(alkyl)-2-(thio) pseudouracil; 5-(alkyl)-2,4 (dithio) pseudouracil; 5-(alkyl)-4 (thio) pseudouracil; 5-(alkyl) pseudouracil; 5-(alkyl) uracil; 5-(alkynyl) uracil; 5-(allylamino) uracil; 5-(cyanoalkyl) uracil; 5-(dialkylaminoalkyl) uracil; 5-(dimethylaminoalkyl) uracil; 5-(guanidiniumalkyl) uracil; 5-(halo) uracil; 5-(1,3-diazole-1-alkyl) uracil; 5-(methoxy) uracil; 5-(methoxycarbonylmethyl)-2-(thio) uracil; 5-(methoxycarbonyl-methyl) uracil; 5-(methyl) 2 (thio) uracil; 5-(methyl) 2,4 (dithio) uracil; 5-(methyl) 4 (thio) uracil; 5-(methyl)-2-(thio) pseudouracil; 5-(methyl)-2,4 (dithio) pseudouracil; 5-(methyl)-4 (thio) pseudouracil; 5-(methyl) pseudouracil; 5-(methylaminomethyl)-2 (thio) uracil; 5-(methylaminomethyl)-2,4 (dithio) uracil; 5-(methylaminomethyl)-4-(thio) uracil; 5-(propynyl) uracil; 5-(trifluoromethyl) uracil; 5-aminoallyl-uridine; 5-bromo-uridine; 5-iodo-uridine; 5-uracil; 6 (azo) uracil; 6-(azo) uracil; 6-aza-uridine; allyamino-uracil; aza uracil; deaza uracil; N3 (methyl) uracil; Pseudo-UTP-1-2-cthanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (+) 1-(2-Hydroxypropyl) pseudouridine TP; (2R)-1-(2-Hydroxypropyl) pseudouridine TP; (2S)-1-(2-Hydroxypropyl) pseudouridine TP; (E)-5-(2-Bromo-vinyl) ara-uridine TP; (E)-5-(2-Bromo-vinyl) uridine TP; (Z)-5-(2-Bromo-vinyl) ara-uridine TP; (Z)-5-(2-Bromo-vinyl) uridine TP; 1-(2,2,2-Trifluoroethyl)-pseudo-UTP; 1-(2,2,3,3,3-Pentafluoropropyl) pseudouridine TP; 1-(2,2-Diethoxyethyl) pseudouridine TP; 1-(2,4,6-Trimethylbenzyl) pseudouridine TP; 1-(2,4,6-Trimethyl-benzyl) pseudo-UTP; 1-(2,4,6-Trimethyl-phenyl) pseudo-UTP; 1-(2-Amino-2-carboxyethyl) pseudo-UTP; 1-(2-Amino-ethyl) pseudo-UTP; 1-(2-Hydroxyethyl) pseudouridine TP; 1-(2-Methoxyethyl) pseudouridine TP; 1-(3,4-Bis-trifluoromethoxybenzyl) pseudouridine TP; 1-(3,4-Dimethoxybenzyl) pseudouridine TP; 1-(3-Amino-3-carboxypropyl) pseudo-UTP; 1-(3-Amino-propyl) pseudo-UTP; 1-(3-Cyclopropyl-prop-2-ynyl) pseudouridine TP; 1-(4-Amino-4-carboxybutyl) pseudo-UTP; 1-(4-Amino-benzyl) pseudo-UTP; 1-(4-Amino-butyl) pseudo-UTP; 1-(4-Amino-phenyl) pseudo-UTP; 1-(4-Azidobenzyl) pseudouridine TP; 1-(4-Bromobenzyl) pseudouridine TP; 1-(4-Chlorobenzyl) pseudouridine TP; 1-(4-Fluorobenzyl) pseudouridine TP; 1-(4-Iodobenzyl) pseudouridine TP; 1-(4-Methanesulfonylbenzyl) pseudouridine TP; 1-(4-Methoxybenzyl) pseudouridine TP; 1-(4-Methoxy-benzyl) pseudo-UTP; 1-(4-Methoxy-phenyl) pseudo-UTP; 1-(4-Methylbenzyl) pseudouridine TP; 1-(4-Methyl-benzyl) pseudo-UTP; 1-(4-Nitrobenzyl) pseudouridine TP; 1-(4-Nitro-benzyl) pseudo-UTP; 1 (4-Nitro-phenyl) pseudo-UTP; 1-(4-Thiomethoxybenzyl) pseudouridine TP; 1-(4-Trifluoromethoxybenzyl) pseudouridine TP; 1-(4-Trifluoromethylbenzyl) pseudouridine TP; 1-(5-Amino-pentyl) pseudo-UTP; 1-(6-Amino-hexyl) pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl} pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl) pseudo-UTP; 1-Methyl-6-(4-morpholino)-pseudo-UTP; 1-Methyl-6-(4-thiomorpholino)-pseudo-UTP; 1-Methyl-6-(substituted phenyl) pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-cthylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridin TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl) uridine TP; 3-Alkyl-pseudo-UTP; 4′-Azidouridine TP; 4′-Carbocyclic uridine TP; 4′-Ethynyluridine TP; 5-(1-Propynyl) ara-uridine TP; 5-(2-Furanyl) uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluorocthyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}] propionic acid; Pseudouridine TP 1-[3-{2-(2-[2-{2 (2-ethoxy)-ethoxy}-ethoxy]-ethoxy)-ethoxy}] propionic acid; Pseudouridine TP 1-[3-{2-(2-[2-ethoxy]-ethoxy)-ethoxy}] propionic acid; Pseudouridine TP 1-[3-{2-(2-ethoxy)-ethoxy}] propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosinc; 2,6-(diamino) purine; 1-(aza)-2-(thio)-3-(aza)-phenoxazin-1-yl: 1,3-(diaza)-2-(oxo)-phenthiazin-1-yl; 1,3-(diaza)-2-(oxo)-phenoxazin-1-yl;1,3,5-(triaza)-2,6-(dioxa)-naphthalene;2 (amino) purine;2,4,5-(trimethyl)phenyl;2′ methyl, 2′amino, 2′azido, 2′fluro-cytidine;2′ methyl, 2′amino, 2′azido, 2′fluro-adenine;2′methyl, 2′amino, 2′azido, 2′fluro-uridine;2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl) isocarbostyrilyl; 3-(methyl) isocarbostyrilyl; 4-(fluoro)-6-(methyl)benzimidazole; 4-(methyl)benzimidazole; 4-(methyl) indolyl; 4,6-(dimethyl) indolyl; 5 nitroindole; 5 substituted pyrimidines; 5-(methyl) isocarbostyrilyl; 5-nitroindole; 6-(aza)pyrimidine; 6-(azo) thymine; 6-(methyl)-7-(aza) indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza) indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl) isocarbostyrilyl; 7-(propynyl) isocarbostyrilyl, propynyl-7-(aza) indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; 06-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza) indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl) uridine TP; and N6-(19-Amino-pentaoxanonadecyl) adenosine TP.

In some embodiments, polynucleotides (e.g., RNA polynucleotides, such as mRNA polynucleotides) include a combination of at least two (e.g., 2, 3, 4 or more) of the aforementioned modified nucleobases.

In some embodiments, modified nucleobases in polynucleotides (e.g., RNA polynucleotides, such as mRNA polynucleotides) are selected from the group consisting of pseudouridine (ψ), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, polynucleotides (e.g., RNA polynucleotides, such as mRNA polynucleotides) include a combination of at least two (e.g., 2, 3, 4 or more) of the aforementioned modified nucleobases.

In some embodiments, one or more polynucleotide of the invention is modified by replacing one or more uridine residue with one or more modified nucleobase. In certain embodiments, at least 50% of the uridine residues of the mRNA polynucleotide are replaced with N1-methylpseudouridine. In certain embodiments, between 50% and 55%, between 55% and 60%, between 60% and 65%, between 65% and 70%, between 70% and 75%, between 75% and 80%, 80% and 85%, between 85% and 90%, between 90% and 95%, and between 95% and 100% of the uridine residues of the mRNA polynucleotide are replaced with N1-methylpsuedouridine. In certain embodiments, 90% of the uridine residues of the mRNA polynucleotide are replaced with N1-methylpsuedouridine. In certain embodiments, 91% of the uridine residues of the mRNA polynucleotide are replaced with N1-methylpsuedouridine. In certain embodiments, 92% of the uridine residues of the mRNA polynucleotide are replaced with N1-methylpsuedouridine. In certain embodiments, 93% of the uridine residues of the mRNA polynucleotide are replaced with N1-methylpsuedouridine. In certain embodiments, 94% of the uridine residues of the mRNA polynucleotide are replaced with N1-methylpsuedouridine. In certain embodiments, 95% of the uridine residues of the mRNA polynucleotide are replaced with N1-methylpsuedouridine. In certain embodiments, 96% of the uridine residues of the mRNA polynucleotide are replaced with N1-methylpsuedouridine. In certain embodiments, 97% of the uridine residues of the mRNA polynucleotide are replaced with N1-methylpsuedouridine. In certain embodiments, 98% of the uridine residues of the mRNA polynucleotide are replaced with N1-methylpsuedouridine. In certain embodiments, 99% of the uridine residues of the mRNA polynucleotide are replaced with N1-methylpsuedouridine In certain embodiments, 100% of the uridine residues of the mRNA polynucleotide are replaced with N1-methylpsuedouridine.

In some embodiments, modified nucleobases in polynucleotides (e.g., RNA polynucleotides, such as mRNA polynucleotides) are selected from the group consisting of 1-methyl-pseudouridine (m¹ψ), 5-methoxy-uridine (mo⁵U), 5-methyl-cytidine (m⁵C), pseudouridine (ψ), α-thio-guanosine and α-thio-adenosine. In some embodiments, polynucleotides includes a combination of at least two (e.g., 2, 3, 4 or more) of the aforementioned modified nucleobases.

In some embodiments, polynucleotides (e.g., RNA polynucleotides, such as mRNA polynucleotides) comprise pseudouridine (ψ) and 5-methyl-cytidine (m5C). In some embodiments, polynucleotides (e.g., RNA polynucleotides, such as mRNA polynucleotides) comprise 1-methyl-pseudouridine (mlv). In some embodiments, polynucleotides (e.g., RNA polynucleotides, such as mRNA polynucleotides) comprise 1-methyl-pseudouridine (m¹ψ) and 5-methyl-cytidine (m5C). In some embodiments, polynucleotides (e.g., RNA polynucleotides, such as mRNA polynucleotides) comprise 2-thiouridine (s2U). In some embodiments, polynucleotides (e.g., RNA polynucleotides, such as mRNA polynucleotides) comprise 2-thiouridine and 5-methyl-cytidine (m5C). In some embodiments, polynucleotides (e.g., RNA polynucleotides, such as mRNA polynucleotides) comprise methoxy-uridine (mo5U). In some embodiments, polynucleotides (e.g., RNA polynucleotides, such as mRNA polynucleotides) comprise 5-methoxy-uridine (mo5U) and 5-methyl-cytidine (m5C). In some embodiments, polynucleotides (e.g., RNA polynucleotides, such as mRNA polynucleotides) comprise 2′-O-methyl uridine. In some embodiments polynucleotides (e.g., RNA polynucleotides, such as mRNA polynucleotides) comprise 2′-O-methyl uridine and 5-methyl-cytidine (m5C). In some embodiments, polynucleotides (e.g., RNA polynucleotides, such as mRNA polynucleotides) comprise N6-methyl-adenosine (m6A). In some embodiments, polynucleotides (e.g., RNA polynucleotides, such as mRNA polynucleotides) comprise N6-methyl-adenosine (m6A) and 5-methyl-cytidine (m5C).

In some embodiments, polynucleotides (e.g., RNA polynucleotides, such as mRNA polynucleotides) are uniformly modified (e.g., fully modified, modified throughout the entire sequence) for a particular modification. For example, a polynucleotide can be uniformly modified with 5-methyl-cytidine (m⁵C), meaning that all cytosine residues in the mRNA sequence are replaced with 5-methyl-cytidine (m⁵C). Similarly, a polynucleotide can be uniformly modified for any type of nucleoside residue present in the sequence by replacement with a modified residue such as those set forth above.

Exemplary nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), and 2-thio-5-methyl-cytidine.

In some embodiments, a modified nucleobase is a modified uridine. Exemplary nucleobases and nucleosides having a modified cytosine, or, a modified uridine include 5-cyano uridine, and 4′-thio uridine.

In some embodiments, a modified nucleobase is a modified adenine. Exemplary nucleobases and nucleosides having a modified adenine include 7-deaza-adenine, 1-methyl-adenosine (m1A), 2-methyl-adenine (m2A), and N6-methyl-adenosine (m6A).

In some embodiments, a modified nucleobase is a modified guanine. Exemplary nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1G), 8-oxo-guanosine, 7-methyl-8-oxo-guanosine.

The polynucleotides of the present disclosure may be partially or fully modified along the entire length of the molecule. For example, one or more or all or a given type of nucleotide (e.g., purine or pyrimidine, or any one or more or all of A, G, U, C) may be uniformly modified in a polynucleotide of the invention, or in a given predetermined sequence region thereof (e.g., in the mRNA including or excluding the poly(A) tail). In some embodiments, all nucleotides X in a polynucleotide of the present disclosure (or in a given sequence region thereof) are modified nucleotides, wherein X may any one of nucleotides A, G, U, C, or any one of the combinations A+G, A+U, A+C, G+U, G+C, U+C, A+G+U, A+G+C, G+U+C or A+G+C.

The polynucleotide may contain from about 1% to about 100% modified nucleotides (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). It will be understood that any remaining percentage is accounted for by the presence of unmodified A, G, U, or C.

The polynucleotides may contain at a minimum 1% and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the polynucleotides may contain a modified pyrimidine such as a modified uracil or cytosine. In some embodiments, at least 5%, at least 10%, at least 25%, at least 50%, at least 80%, at least 90% or 100% of the uracil in the polynucleotide is replaced with a modified uracil (e.g., a 5-substituted uracil). The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the polynucleotide is replaced with a modified cytosine (e.g., a 5-substituted cytosine). The modified cytosine can be replaced by a compound having a single unique structure, or can be replaced by a plurality of compounds having different structures (e.g., 2, 3, 4 or more unique structures).

In Vitro Transcription (IVT) of RNA (e.g., mRNA)

“In vitro transcription (IVT)” is a method to synthesize mRNA from a linear DNA template using an RNA polymerase, where the RNA polymerase binds to a promoter sequence and transcribes mRNA through the addition of complimentary nucleotides in the 5′ to 3′ direction. Norovirus vaccines of the present disclosure comprise at least one RNA polynucleotide, such as a mRNA (e.g., modified mRNA). mRNA, for example, is transcribed in vitro from template DNA, referred to as an “in vitro transcription template.” In some embodiments, the at least one RNA polynucleotide has at least one chemical modification. The at least one chemical modification may include, but is expressly not limited to, any modification described herein.

Linear DNA templates encoding for mRNA sequences are prepared from purified plasmid DNA and may contain an RNA polymerase promoter sequence, encoded 5′ UTR, coding sequence, encoded 3′ UTR with a poly(A) tail, and a lincarization cut site. Plasmid DNA sequences are generated using standard cloning methods in a plasmid vector backbone, such as pUC19, pUC57, or pmRNAXP, and plasmid DNA is purified from fermentation of transformed E. coli strains typically used for plasmid production, such as Stable or DH5a, using standard methods (Scc, e.g., US Patent Application Publication No. 2019/0083602, US Patent Application Publication No. 2020/0392518). mRNA may be produced in an IVT reaction with a co-transcriptional cap analog addition and a DNA template encoded poly(A) tail, or the cap and/or poly(A) tail may be added enzymatically after the IVT reaction. DNA plasmids may contain an adenine-guanine (AG) sequence at the start of the 5′ UTR after a T7 promoter sequence to enable co-transcriptional capping with a Cap 1 AG analog (m7G (5′) ppp (5′) (2′OMcA) pG). DNA plasmids may also contain a specific restriction enzyme cut site after an encoded 3′ poly(A) tail. Plasmids may be linearized in a digestion reaction containing purified plasmid DNA at a concentration of about 0.1-2 mg/mL and the appropriate restriction enzyme, such as BbsI or BspQI, at a concentration of about 500-20,000 U/mg in a digest mixture that may contain tris hydrochloride, magnesium, potassium, acetate, albumen, and/or other excipients at a solution pH of about 7-8. The reaction is incubated at a temperature range of about 34-40° C., preferably 37° C., for about 30-90 min, preferably 60 min.

The linear DNA template may be purified by solvent extraction, alcohol precipitation, centrifugation, chromatographic, and/or filtration-based methods to remove the uncut plasmid DNA, restriction enzyme, and digest mixture components. Purified linear DNA templates may be prepared at a concentration of about 0.5-2 mg/mL, preferably 1 mg/mL, in water or a buffer containing tris hydrochloride, ethylenediaminetetraacetic acid (EDTA), and/or other excipient at a solution pH of about 7-8.

The IVT reaction is performed in a temperature-controlled reaction vessel with an incubation temperature range of about 34-40° C., preferably 37° C. The linear DNA template may be added to the IVT reaction mixture at a concentration range of about 10-200 μg/mL, preferably 40-60 μg/mL. Natural nucleoside triphosphates (NTPs) including adenosine triphosphate (ATP), guanosine triphosphate (GTP), cytidine triphosphate (CTP), uridine triphosphate (UTP), or modified NTPs, such as pseudouridine (Y), N1-methylpseudouridine (m1\′), N⁶-methyladenosine (m6A), or N5-methylcytidine (m⁵C), may be used in the IVT reaction, preferably ATP, GTP, CTP, and mlY′. The NTPs may be incorporated in mRNA as a mixture of natural and substituted modified NTPs, such as a mixture of 50% UTP and 50% mly′. Preferably, 100% mlY is added to fully substitute for UTP. The NTPs may be added to the IVT reaction mixture at a concentration range of about 2-15 mM, or preferably 8-14 mM for ATP, GTP, CTP and 4-7 mM for mlY. A 5′ Cap 1 AG analog, such as CleanCap AG (3′OMe) (m7 (3′OMeG) (5′) ppp (5′) (2′OMeA) pG) or CleanCap AG (m7 (5′) ppp (5′) (2′OMeA) pG), preferably CleanCap AG (3′OMe), may be added to the IVT reaction mixture at a concentration range of about 1-10 mM, preferably 3-5 mM. An RNA polymerase, such as T7 RNA polymerase, may be added to the IVT reaction mixture at a concentration range of about 2,000-20,000 U/mL, preferably 8,000-12,000 U/mL.

A pyrophosphatase, such as yeast inorganic pyrophosphatase, and RNase inhibitor, such as murine RNase inhibitor, may be added to the IVT reaction mixture at a concentration range of 1-5 U/mL and 500-2,000 U/mL, respectively. Other components of the IVT reaction mixture may include tris hydrochloride, magnesium, spermidine, dithiothreitol (DTT), sodium, chloride, potassium, acetate, phosphate, polysorbate-20, polysorbate-80, TritonX-100, glycerol and/or other excipients at a final solution pH of about 6-9. The IVT reaction mixture is typically incubated with mixing for 1-6 hr, preferably 3-4 hr. Preferably, the mRNA is produced in the IVT reaction with a co-transcriptional 5′ Cap 1 AG analog and a DNA template encoded 3′ poly(A) tail and no additional enzymatic capping or poly(A) tail addition reactions are necessary. The IVT reaction is terminated by addition of a DNase, such as DNase I, to digest the linear DNA template into small oligonucleotides and stop transcription. DNase is added to the IVT reaction mixture at a concentration range of about 100-500 U/mL, preferably 350 U/mL, along with calcium chloride at a concentration range of about 1-5 mM, preferably 3.5 mM, and incubated at about 34-40° C., preferably 37° C. for 0.5-3 hr, preferably 1 hr. After DNasc digestion, a proteinase, such as Proteinase K, may be added to the IVT reaction mixture to digest enzymes used in the IVT reaction into small peptides. Proteinase is added to the IVT reaction mixture at a concentration range of about 0.05-0.2 mg/mL, preferably 0.1 mg/mL, along with sodium dodecyl sulfate (SDS) and additional DTT, and incubated at about 34-40° C., preferably 37° C. for 0.5-3 hr, preferably 1 hr. Preferably, only DNase treatment is performed, and no proteinase treatment is performed. After DNase or DNase followed by proteinase treatments are complete, the IVT reaction mixture is adjusted to 10-100 mM EDTA, preferably 50 mM to quench enzyme activity.

The resulting IVT reaction mixture contains the target mRNA sample and contaminants such as template DNA and digested oligonucleotides, enzymes including RNA polymerase, pyrophosphatase, RNase inhibitor, DNase, and proteinase, small molecules including frec nucleotides, pyrophosphate, magnesium, spermidine, and/or other IVT reaction matrix excipients, and RNA-related contaminants such as RNA fragments, double-stranded RNA, uncapped RNA, or RNA lacking a poly(A) tail.

An initial tangential flow filtration (TFF) step may be performed after DNase and proteinase treatment for buffer exchange and clearance of small impurities. TFF is operated by pumping a feed solution across a membrane at a transmembrane pressure (TMP) of about 1-10 psi, where solution components larger than the molecular weight cut off (MWCO) remain in the retentate, and components smaller than the MWCO may be permeated through the membranc. The membrane MWCO may range from 30-500 kDa in a flat sheet or hollow fiber format and may be composed of a variety of materials including polyethersulfone (PES), polysulfonc (PS), or regenerated cellulose (RC). When the IVT reaction mixture is processed across TFF, the large mRNA molecules are retained while digested impurities and other small molecules are cleared in the permeate. Prior to starting TFF, the IVT reaction mixture may be diluted 2-20-fold in water or a buffer containing tris hydrochloride, sodium phosphate, or sodium citrate at a concentration of about 1-100 mM, preferably 10 mM tris hydrochloride, at a pH of about 6-8 and EDTA at a concentration of about 1-10 mM, preferably 2 mM. A TFF membrane loaded with approximately 1-25 g mRNA per m2 of membrane area of diluted IVT reaction mixture operated at a TMP of about 3-7 psi and crossflow shear rate of about 2000-12000 s⁻¹, may be used to buffer exchange the diluted IVT mixture across 5-15 diafiltration volumes (DVs) into water or a buffer containing tris hydrochloride, sodium phosphate, or sodium citrate at a concentration of about 1-100 mM at a pH of about 6-8 and EDTA at a concentration of about 1-10 mM. Preferably, a 50 kDa hollow fiber PES membrane loaded with 5-10 g-mRNA/m² operated at a TMP of about 4 psi and a crossflow shear rate of 4000 s⁻¹ is used to buffer exchange the diluted IVT mixture across 10 DVs into 10 mM tris hydrochloride, 2 mM EDTA, pH 7.2.

The purified mRNA-containing solution in the TFF retentate may be forwarded to hybridization affinity chromatography media using an oligo deoxythymine (oligo dT) ligand conjugated to a stationary phase to selectively bind mRNA through complimentary base pairing of the oligo dT ligand with the mRNA poly(A) tail. IVT reaction contaminants including RNA polymerase, pyrophosphatase, RNase inhibitor, DNase, and proteinase, small molecules including free nucleotides, pyrophosphate, magnesium, spermidine, other IVT matrix excipients, and RNA fragments lacking a poly(A) tail are not expected to bind to the oligo dT ligand. Preferably, the DNase treated IVT reaction mixture is forwarded directly to oligo dT affinity chromatography, omitting the Proteinase K and initial TFF steps. The oligo dT ligand may consist of about 15-30 deoxythymine bases connected to a linker coupling the ligand to a support surface, such as a microporous polymethacrylate monolith, crosslinked poly(styrene-divinylbenzene) bead, or electrospun cellulose nanofibers. The mRNA-containing solution forwarded to the oligo dT hybridization affinity chromatography step may be diluted about 2-50-fold into an oligo dT binding matrix consisting of a salt, such as sodium chloride, potassium chloride, lithium chloride, guanidine hydrochloride, or other similar salts, at a concentration of about 100-1000 mM, a buffer, such as tris hydrochloride, sodium phosphate, or sodium citrate, at a concentration of 5-100 mM at a pH of about 6-8, and EDTA at a concentration of about 1-10 mM. Preferably, the DNase treated IVT mixture is diluted about 12-fold into an oligo dT binding matrix consisting of 400 mM sodium chloride, 10 mM tris hydrochloride, 2 mM EDTA, pH 7.2. The mRNA-containing solution may be pumped through oligo dT chromatography media with an approximate loading of about 1-8 mg/mL-media at a residence time of about 0.1-10 min, and upon binding of mRNA with a poly(A) tail, the column is washed with 2-10 column volumes (CV) of a mobile phase consisting of a salt, such as sodium chloride, potassium chloride, lithium chloride, guanidine hydrochloride, or other similar salts, at a concentration of about 10-200 mM, a buffer, such as tris hydrochloride, sodium phosphate, or sodium citrate, at a concentration of 5-100 mM at a pH of about 6-8, and EDTA at a concentration of about 1-10 mM. Preferably, the mRNA containing mixture is pumped through a microporous polymethacrylate monolith media at loading of 2-3 mg/mL-media and a residence time of 0.5 min, and the column is washed with 5 CVs of a buffer consisting of 50 mM sodium chloride, 10 mM tris hydrochloride, 2 mM EDTA, pH 7.2. The bound mRNA is cluted from the oligo dT ligand with 2-10 CVs of a low ionic strength mobile phase consisting of water or a buffer, such as tris hydrochloride, sodium phosphate, or sodium citrate, at a concentration of 1-20 mM at a pH of about 6-8. Preferably, the bound mRNA is cluted with 4-5 CVs of 10 mM tris hydrochloride, pH 7.2. The oligo dT chromatography media may be regenerated with sodium hydroxide and re-used for subsequent chromatography purification cycles. Preferably, a polymethacrylate monolith is be regenerated with 0.5 M sodium hydroxide and reused through about 2-6 purification cycles of the same DNase treated IVT mixture diluted in oligo dT binding buffer.

The mRNA-containing oligo dT hybridization affinity chromatography elution fraction may be purified by polishing chromatographic methods to remove residual RNA-related impurities, such as RNA fragments, double-stranded mRNA, and uncapped RNA, or residual enzymes such as RNA polymerase. Examples of polishing chromatographic methods may include mixed-mode chromatography, hydrophobic interaction chromatography, anion-exchange chromatography, reversed-phase chromatography, ceramic hydroxyapatite chromatography, size-exclusion chromatography, or cellulose chromatography. Preferably, no additional polishing purification methods are performed after oligo dT chromatography.

The mRNA-containing oligo dT hybridization affinity chromatography elution fraction is buffer exchanged and purified by a final TFF that may be operated similarly to the previously described initial TFF. The final TFF membrane may be operated at a TMP of 1-10 psi using a 30-500 kDa MWCO membrane in a flat sheet or hollow fiber format and may be composed polyethersulfone (PES), polysulfone (PS), or regenerated cellulose (RC). A 50-100 kDa hollow fiber PES membrane loaded with approximately 1-20 g mRNA per m2 of membrane area operated at a TMP of 3-7 psi and crossflow shear rate of about 2000-12000 s⁻¹ may be used to concentrate the oligo dT elution fraction approximately 2-20-fold followed by a buffer exchange across 4-15 DVs into water or a buffer consisting of about 1-10 mM tris hydrochloride, sodium phosphate, or sodium citrate at a pH of about 5-8. Preferably, a 50 kDa hollow fiber PES membrane loaded with 5-10 g-mRNA/m² operated at a TMP of about 4 psi and a crossflow shear rate of 4000 s⁻¹ is used to concentrate 8-fold followed by a buffer exchange of 6 DVs into 1 mM sodium citrate, pH 6.4.

The mRNA-containing retentate from the final TFF is filtered at a flux of about 25-1000 L/m2-hr and a loading of about 10-1000 g/m² through a bioburden reduction filter with a nominal pore size of about 0.2 μm. The filter may be composed of a variety of materials including hydrophilic polyvinylidene fluoride (PVDF), PES, or cellulose acetate and may contain a prefilter with a nominal pore size ranging from about 0.2-1 μm. Preferably, the final TFF retentate is pumped at a flux of 300 L/m²-hr and a loading of 100 g/m² through a 0.2 μm PVDF filter. The concentration of mRNA in the bioburden reduction filter product is calculated from a sample measurement of absorbance at 260 nm using a spectrophotometer. The filtration product may be diluted with the final TFF diafiltration buffer to a target a final concentration of mRNA ranging from about 0.5-5 mg/mL. Preferably, the filtration product is diluted with 1 mM sodium citrate, pH 6.4 to a mRNA concentration of about 1 mg/mL. The purified mRNA may be stored at refrigerated conditions at about 2-8° C. or frozen at about −20° C. or <−60° C.

### Antigenic Polypeptides

An “antigenic polypeptide” is a polypeptide which induces an immune response when administered to an animal, preferably a mammal. In some embodiments, an antigenic polypeptide includes gene products, naturally occurring polypeptides, synthetic polypeptides, homologs, orthologs, paralogs, fragments and other equivalents, variants, and analogs of the foregoing. In certain embodiments, the antigenic polypeptide is a norovirus VP1 or VP2 polypeptide. In certain embodiments, the antigenic polypeptide is any one of SEQ ID NOs: 14-18. Antigenic polypeptides of the present disclosure may be prepared by using any suitable method known in the art, including synthetic methods such as e.g., solid phase synthesis, as well as recombinant and in vitro methods, such as in vitro transcription reactions. In certain embodiments, antigenic polypeptides may be prepared by in vitro transcription of the polynucleotides disclosed herein.

A “variant” is a molecule that differs in its amino or nucleic acid sequence relative to a native sequence or a reference sequence. Sequence variants may possess substitutions, deletions, insertions, or a combination of any two or three of the foregoing, at certain positions within the amino or nucleic acid sequence, as compared to a native sequence or a reference sequence. Ordinarily, variants possess at least 50% identity to a native sequence or a reference sequence. In some embodiments, variants share at least 80% identity or at least 90% identity with a native sequence or a reference sequence.

The present disclosure provides several types of compositions that are polynucleotide or polypeptide based, including variants and derivatives. These include, for example, substitutional, insertional, deletion, and covalent variants and derivatives. The term “derivative” is synonymous with the term “variant” and generally refers to a molecule that has been modified and/or changed in any way relative to a reference molecule or a starting molecule. As such, polynucleotides encoding peptides or polypeptides containing substitutions, insertions, and/or additions, deletions, and covalent modifications with respect to reference sequences, in particular the polypeptide sequences disclosed herein, are included within the scope of this disclosure.

“Substitutional variants” when referring to polynucleotides, are those that have at least one nucleotide residue in a native or starting sequence removed and a different nucleotide inserted in its place at the same position. In some embodiments, one or more uridine residues in an mRNA polynucleotide of the present disclosure are replaced with a pseudouridine. The modification of mRNA nucleosides, e.g., with pseudouridine or a pseudouridine derivative, have been described previously. See International Patent Application Publication WO 2007/024708.

As used herein the terms “termini” or “terminus” when referring to polypeptides or polynucleotides refers to an extremity of a polypeptide or polynucleotide respectively. Such extremity is not limited only to the first or final site of the polypeptide or polynucleotide but may include additional amino acids or nucleotides in the terminal regions.

Polypeptide or polynucleotide molecules of the present disclosure may share a certain degree of sequence similarity or identity with the reference molecules (e.g., reference polypeptides or reference polynucleotides), for example, with art-described molecules (e.g., engineered or designed molecules or wild-type molecules). The term “identity,” as known in the art, refers to a relationship between the sequences of two or more polypeptides or polynucleotides, as determined by comparing the sequences. In the art, identity also means the degree of sequence relatedness between two sequences as determined by the number of matches between strings of two or more amino acid residues or nucleic acid residues. Identity measures the percent of identical matches between the smaller of two or more sequences with gap alignments (if any) addressed by a particular mathematical model or computer program (e.g., “algorithms”). Identity of related peptides can be readily calculated by known methods. “% identity” as it applies to polypeptide or polynucleotide sequences is defined as the percentage of residues (amino acid residues or nucleic acid residues) in the candidate amino acid or nucleic acid sequence that are identical with the residues in the amino acid sequence or nucleic acid sequence of a second sequence after aligning the sequences and introducing gaps, if necessary, to achieve the maximum percent identity. Methods and computer programs for the alignment are well known in the art. Identity depends on a calculation of percent identity but may differ in value due to gaps and penalties introduced in the calculation. Generally, variants of a particular polynucleotide or polypeptide have at least 40%, 45%, 50%, 55%, 60%, 65%, 70%, 75%, 80%, 85%, 90%, 91%, 92%, 93%, 94%, 95%, 96%, 97%, 98%, 99%, but less than 100% sequence identity to that particular reference polynucleotide or polypeptide as determined by sequence alignment programs and parameters described herein and known to those skilled in the art. Such tools for alignment include those of the BLAST suite. See Stephen F. Altschul, et al. (1997). “Gapped BLAST and PSI-BLAST: a new generation of protein database search programs,” Nucleic Acids Res. 25:3389-3402. Another popular local alignment technique is based on the Smith-Waterman algorithm. See Smith, T. F. & Waterman, M. S. (1981) “Identification of common molecular subsequences.” J. Mol. Biol. 147:195-197. A general global alignment technique based on dynamic programming is the Needleman-Wunsch algorithm. Sec Needleman, S. B. & Wunsch, C. D. (1970) “A general method applicable to the search for similarities in the amino acid sequences of two proteins.” J. Mol. Biol. 48:443-453. More recently, a Fast Optimal Global Sequence Alignment Algorithm (FOGSAA) was developed that purportedly produces global alignment of nucleotide and protein sequences faster than other optimal global alignment methods, including the Needleman-Wunsch algorithm.

### Lipid Nanoparticles (LNPs)

As used herein, “lipid nanoparticle” or “LNP” refers to any lipid composition that can be used to deliver a product, including, but not limited to, liposomes or vesicles, wherein an aqueous volume is encapsulated by amphipathic lipid bilayers (e.g., single; unilamellar or multiple; multilamellar), or, in other embodiments, wherein the lipids coat an interior comprising a prophylactic or therapeutic product, or lipid aggregates or micelles, wherein the lipid encapsulated prophylactic or therapeutic product is contained within a relatively disordered lipid mixture. In certain embodiments, mRNA polynucleotides of the present invention may be encapsulated together in a LNP comprising one or more cationic or polycationic compounds. Except where noted, the LNP does not need to have an antigenic polypeptide incorporated therein and may be used to deliver a product (i.e., mRNA polynucleotides) to a mammal when the LNP and mRNA polynucleotides in the same formulation.

As used herein, “polyamine” means compounds having two or more amino groups. Examples include putrescine, cadaverine, spermidine, and spermine.

Unless otherwise specified, mole % refers to a mole percent of total lipids.

Generally, the LNPs of the compositions of the invention are composed of one or more cationic lipids (including ionizable cationic lipids) and one or more poly(ethyleneglycol)-lipids (PEG-lipids). In certain embodiments, the LNPs further comprise one or more non-cationic lipids. The one or more non-cationic lipids can include a phospholipid, phospholipid derivative, a sterol, a fatty acid, or a combination thereof.

Cationic lipids and ionizable cationic lipids suitable for the LNPs are described herein. Ionizable cationic lipids are characterized by the weak basicity of their lipid head groups, which affects the surface charge of the lipid in a pH-dependent manner, rendering them positively charged at acidic pH but close to charge-neutral at physiologic pH. Cationic lipids are characterized by monovalent or multivalent cationic charge on their headgroups, which renders them positively charged at neutral pH. In certain embodiments, the cationic and ionizable lipid is capable of complexing with hydrophilic bioactive molecules to produce a hydrophobic complex that partitions into the organic phase of a two-phase aqueous/organic system. It is contemplated that both monovalent and polyvalent cationic lipids may be utilized to form hydrophobic complexes with bioactive molecules.

Preferred cationic and ionizable cationic lipids for use in forming the LNPs include, but are not limited to, N,N-dioleyl-N,N-dimethylammonium chloride (“DODAC”); N-(2,3dioleyloxy) propyl)-N,N,Ntrimethylammonium chloride (“DOTMA”); N,NdistearylN,N-dimethylammonium bromide (“DDAB”); N-(2,3diolcoyloxy) propyl)-N,N,N-trimethylamntonium chloride (“DODAP”); 1,2 bis (oleoyloxy)-3-(trimethylammonio) propane (DOTAP); 3-(N—(N,N-dimethylaminocthanc)-carbam-oyl) cholesterol (“DC-Chol”); diheptadecylamidoglycylspermidine (“DHGS”) and N-(1,2-dimyristyloxyprop-3-yl)-N,N-dimethyl-N-hydoxyethyl ammonium bromide (“DMRIE”). Additionally, a number of commercial preparations of cationic lipids, as well as other components, are available which can be used in the present invention. These include, for example, LIPOFECTIN® (commercially available cationic lipid nanoparticles comprising DOTMA and 1,2diolcoyl-sn-3-phosphoethanolamine (“DOPE”), from GIBCOBRL, Grand Island, N.Y., USA); and LIPOFECTAMINE® (commercially available cationic lipid nanoparticles comprising N-(1-(2,3diolcyloxy) propyl)N-(2-(sperminecarboxamido)ethyl)-N,N-dimethylammonium trifluoroacetate (“DOSPA”) and (“DOPE”), from (GIBCOBRL). The following lipids are cationic and have a positive charge at below physiological pH: DODAP, DODMA, DMDMA, 1,2-DiLinoleyloxy-N,N-dimethylaminopropane (DLinDMA), 4-(2,2-diocta-9,12-dienyl-[1,3] dioxolan-4-ylmethyl)-dimethylamine, DLinKDMA (WO 2009/132131), DLin-K-C2-DMA (WO2010/042877), DLin-M-C3-DMA (WO2010/146740 and/or WO2010/105209), DLin-MC3-DMA (heptatriaconta-6,9,28,31-tetraen-19-yl 4-(dimethylamino) butanoate; Jayaraman et al., 2012, Angew. Chem. Int. Ed. Engl. 51:8529-8533), 2-{4-[(3β)-cholest-5-en-3-yloxy]butoxy}-N,N-dimethyl-3-[(9Z,12Z)-octadeca-9,12-dienlyloxyl]propan-1-amine) (CLinDMA), and the like. Other cationic lipids suitable for use in the invention include, e.g., the cationic lipids described in U.S. Pat. Nos. 5,208,036, 5,264,618, 5,279,833 and 5,283,185, and U.S. Patent Application Publication Nos. 2008/0085870 and 2008/0057080. Other cationic lipids suitable for use in the invention include, e.g., Lipids E0001-E0118 or E0119-E0180 as disclosed in Table 6 (pages 112-139) of International Patent Application Publication No. WO2011/076807 (which also discloses methods of making, and methods of using these cationic lipids).

In certain aspects of this embodiment of the invention, the LNPs comprise one or more of the following ionizable cationic lipids: DLinDMA, DlinKC2DMA DLin-MC3-DMA, CLinDMA, or S-Octyl CLinDMA. See International Patent Application Publication No. WO2010/021865.

In certain aspects of this embodiment of the invention, LNPs comprise one or more ionizable cationic lipids described in International Patent Application Publication No. WO2011/022460, or any pharmaceutically acceptable salt thereof, or a stereoisomer of any of the compounds or salts therein.

When structures of the same constitution differ in respect to the spatial arrangement of certain atoms or groups, they are stereoisomers, and the considerations that are significant in analyzing their interrelationships are topological. If the relationship between two stereoisomers is that of an object and its nonsuperimposable mirror image, the two structures are enantiomeric, and each structure is said to be chiral. Stereoisomers also include diastercomers, cis-trans isomers and conformational isomers. Diastereoisomers can be chiral or achiral, and are not mirror images of one another. Cis-trans isomers differ only in the positions of atoms relative to a specified plane in cases where these atoms are, or are considered as if they were, parts of a rigid structure. Conformational isomers are isomers that can be interconverted by rotations about formally single bonds. Examples of such conformational isomers include cyclohexane conformations with chair and boat conformers, carbohydrates, linear alkane conformations with staggered, eclipsed and gauche conformers, etc. Sec J. Org. Chem. 35, 2849 (1970).

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, enantiomers are identical except that they are non-superimposable mirror images of one another. A mixture of enantiomers is often called an enantiomeric mixture. A 50:50 mixture of enantiomers is referred to as a racemic mixture. Many of the compounds described herein can have one or more chiral centers and therefore can exist in different enantiomeric forms. If desired, a chiral carbon can be designated with an asterisk (*). When bonds to the chiral carbon are depicted as straight lines in the Formulas of the invention, it is understood that both the (R) and(S) configurations of the chiral carbon, and hence both enantiomers and mixtures thereof, are embraced within the Formula. As is used in the art, when it is desired to specify the absolute configuration about a chiral carbon, one of the bonds to the chiral carbon can be depicted as a wedge (bonds to atoms above the plane) and the other can be depicted as a series or wedge of short parallel lines (bonds to atoms below the plane). The Cahn-Inglod-Prelog system can be used to assign the (R) or(S) configuration to a chiral carbon.

When the compounds of the present invention contain one chiral center, the compounds exist in two enantiomeric forms and the present invention includes both enantiomers and mixtures of enantiomers, such as the specific 50:50 mixture referred to as a racemic mixtures. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step is required to liberate the desired enantiomeric form. Alternatively, specific enantiomers may be synthesized by asymmetric synthesis using optically active reagents, substrates, catalysts or solvents, or by converting one enantiomer into the other by asymmetric transformation.

Designation of a specific absolute configuration at a chiral carbon of the compounds of the invention is understood to mean that the designated enantiomeric form of the compounds is in enantiomeric excess (ec) or in other words is substantially free from the other enantiomer. For example, the “R” forms of the compounds are substantially free from the “S” forms of the compounds and are, thus, in enantiomeric excess of the “S” forms. Conversely, “S” forms of the compounds are substantially free of “R” forms of the compounds and are, thus, in enantiomeric excess of the “R” forms. Enantiomeric excess, as used herein, is the presence of a particular enantiomer at greater than 50%. In a particular embodiment when a specific absolute configuration is designated, the enantiomeric excess of depicted compounds is at least about 90%.

When a compound of the present invention has two or more chiral carbons it can have more than two optical isomers and can exist in diastereoisomeric forms. For example, when there are two chiral carbons, the compound can have up to 4 optical isomers and 2 pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastercomers. The diastereoisomeric pairs may be separated by methods known to those skilled in the art, for example chromatography or crystallization and the individual enantiomers within each pair may be separated as described above. The present invention includes each diastereoisomer of such compounds and mixtures thereof.

The LNPs may also comprise any combination of two or more of the cationic lipids described herein. In certain aspects, the cationic lipid typically comprises from about 0.1 to about 99.9 mole % of the total lipid present in said particle. In certain aspects, the cationic lipid can comprise from about 80 to about 99.9% mole %. In other aspects, the cationic lipid comprises from about 2% to about 70%, from about 5% to about 50%, from about 10% to about 45%, from about 20% to about 99.8%, from about 30% to about 70%, from about 34% to about 59%, from about 20% to about 40%, or from about 30% to about 40% (mole %) of the total lipid present in said particle.

The LNPs described herein can further comprise a noncationic lipid, which can be any of a variety of neutral uncharged, zwitterionic or anionic lipids capable of producing a stable complex. They are preferably neutral, although they can be negatively charged. Examples of noncationic lipids useful in the present invention include phospholipid-related materials, such as natural phospholipids, synthetic phospholipid derivatives, fatty acids, sterols, and combinations thereof. Natural phospholipids include phosphatidylcholine (PC), phosphatidylethanolamine (PE), and phosphatidylglycerol (PG), phosphatidylserinc (PS), phosphatidylinositol (PI), Phosphatidic acid (phosphatidate) (PA), dipalmitoylphosphatidylcholine, monoacyl-phosphatidylcholine (lyso PC), 1-palmitoyl-2-olcoyl-sn-glycero-3-phosphocholine (POPC), N-Acyl-PE, phosphoinositides, and phosphosphingolipids. Phospholipid derivatives include phosphatidic acid (DMPA, DPPA, DSPA), phosphatidylcholine (DDPC, DLPC, DMPC, DPPC, DSPC, DOPC, POPC, DEPC), phosphatidylglycerol (DMPG, DPPG, DSPG, POPG), phosphatidylethanolamine (DMPE, DPPE, DSPE DOPE), and phosphatidylserine (DOPS). Fatty acids include C14:0, palmitic acid (C16:0), stearic acid (C18:0), oleic acid (C18:1), linoleic acid (C18:2), linolenic acid (C18:3), and arachidonic acid (C20:4), C20:0, C22:0 and lethicin.

In certain embodiments of the invention the non-cationic lipid is selected from: lecithin, phosphatidylethanolamine, lysolecithin, lysophosphatidylethanolamine, phosphatidylserine, phosphatidylinositol, sphingomyelin, cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), diolcoylphosphatidylglycerol (DOPG), dipalmitoylphosphatidylglycerol (DPPG), diolcoyl-phosphatidylethanolamine (DOPE), palmitoylolcoylphosphatidylcholine (POPC), palmitoyloleoyl-phosphatidylet-hanolamine (POPE) and diolcoyl-phosphatidylethanolamine 4-(N-maleimidomethyl)-cyclohexane-1-carboxylate (DOPE-mal). Noncationic lipids also include sterols such as cholesterol, stigmasterol or stigmastanol. Cholesterol is known in the art. See U.S. Patent Application Publication Nos: U.S. 2006/0240554 and U.S. 2008/0020058. In certain embodiments, the LNP comprise a combination of a phospholipid and a sterol.

Where present, the non-cationic lipid typically comprises from about 0.1% to about 65%, about 2% to about 65%, about 10% to about 65%, or about 25% to about 65% expressed as mole percent of the total lipid present in the LNP. The LNPs described herein further include a polyethyleneglycol (PEG) lipid conjugate (“PEG-lipid”) which may aid as a bilayer stabilizing component. The lipid component of the PEG lipid may be any non-cationic lipid described above including natural phospholipids, synthetic phospholipid derivatives, fatty acids, sterols, and combinations thereof. In certain embodiments of the invention, the PEG-lipids include, PEG coupled to dialkyloxypropyls (PEG-DAA) as described in, e.g., International Patent Application Publication No. WO 05/026372, PEG coupled to diacylglycerol (PEG-DAG) as described in, e.g., U.S. Patent Publication Nos. 20030077829 and 2005008689; PEG coupled to phosphatidylethanolamine (PE) (PEG-PE), or PEG conjugated to 1,2-Di-O-hexadecyl-sn-glyceride (PEG-DSG), or any mixture thereof. Sec, e.g., U.S. Pat. No. 5,885,613.

In one embodiment, the PEG-DAG conjugate is a dilaurylglycerol (C12)-PEG conjugate, a PEG dimyristylglycerol (C14) conjugate, a PEG-dipalmitoylglycerol (C16) conjugate, a PEG-dilaurylglycamide (C12) conjugate, a PEG-dimyristylglycamide (C14) conjugate, a PEG-dipalmitoylglycamide (C16) conjugate, or a PEG-disterylglycamide (C18). Those of skill in the art will readily appreciate that other diacylglycerols can be used in the PEG-DAG conjugates.

In certain embodiments, PEG-lipids include, but are not limited to, PEG-dimyristolglycerol (PEG-DMG), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), and PEG-1,2-dimyristyloxlpropyl-3-amine (PEG-c-DMA).

In certain embodiments, the PEG-lipid is PEG coupled to dimyristoylglycerol (PEG-DMG), e.g., as described in Abrams et al., 2010, Molecular Therapy 18 (1): 171, and U.S. Patent Application Publication Nos. US 2006/0240554 and US 2008/0020058.

In certain embodiments, the PEG-lipid, such as a PEG-DAG, PEG-cholesterol, PEG-DMB, comprises a polyethylene glycol having an average molecular weight ranging of about 500 daltons to about 10,000 daltons, of about 750 daltons to about 5,000 daltons, of about 1,000 daltons to about 5,000 daltons, of about 1,500 daltons to about 3,000 daltons or of about 2,000 daltons. In certain embodiments, the PEG-lipid comprises PEG400, PEG1500, PEG2000 or PEG5000.

The acyl groups in any of the lipids described above are preferably acyl groups derived from fatty acids having about C10 to about C24 carbon chains. In one embodiment, the acyl group is lauroyl, myristoyl, palmitoyl, stearoyl or oleoyl.

The PEG-lipid conjugate typically comprises from about 0.1% to about 15%, from about 0.5% to about 20%, from about 1.5% to about 18%, from about 4% to about 15%, from about 5% to about 12%, from about 1% to about 4%, or about 2% expressed as a mole % of the total lipid present in said particle.

In certain embodiments of the invention, the LNPs comprise one or more cationic lipids, cholesterol and 1,2-Dimyristoyl-sn-glycerol methoxypolyethylene glycol (PEG-DMG).

In certain embodiments the invention, the LNPs comprise one or more cationic lipids, cholesterol, 1,2-Distearoyl-sn-glycero-3-phosphocholine (DSPC), and 1,2-Dimyristoyl-sn-glycerol methoxypolyethylene glycol (PEG-DMG).

In some embodiments of the invention, the LNP comprises 34-59 mole % of a cationic lipid, 30-48 mole % of a sterol, 10-24 mole % of a phospholipid, and 1-2 mole % of a PEG-lipid.

In some embodiments, the cationic lipid is selected from the group consisting of:

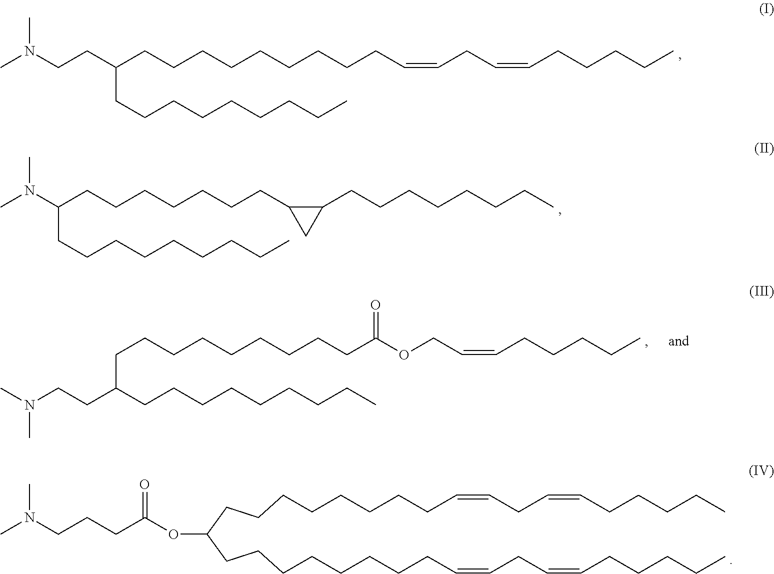

In some embodiments, the sterol is:

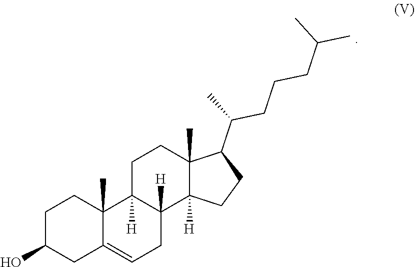

In some embodiments, wherein the phospholipid is selected from the group consisting of:

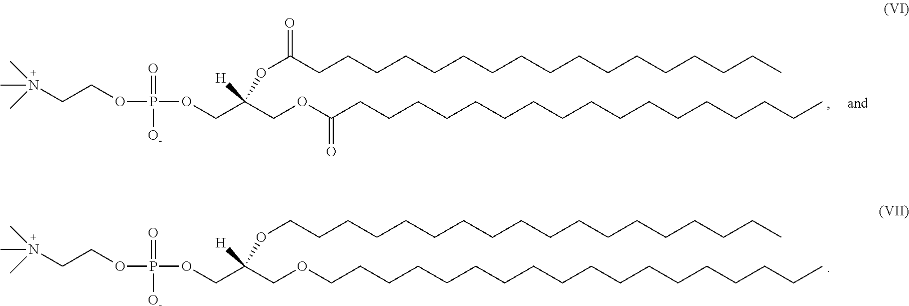

In some embodiments, the polyethyleneglycol-lipid is selected from the group consisting of:

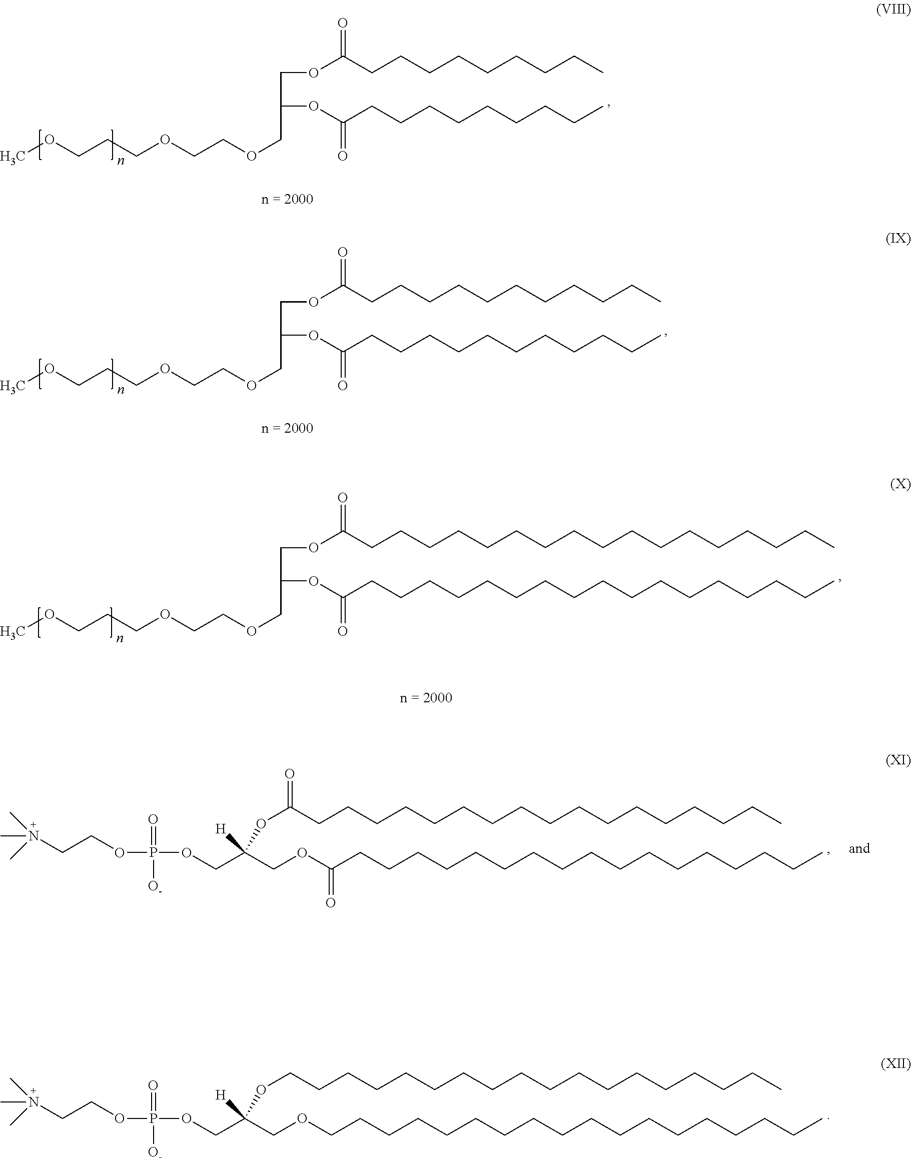

In certain embodiments of the invention, the LNPs comprise lipid compounds assembled within the following molar ratios:
Cationic Lipid (20-99.8 mole %)
Non-cationic lipid (0.1-65 mole %) and
PEG-DMG (0.1-20 mole %).

In certain embodiments of the invention, the LNPs comprise lipid compounds assembled within the following molar ratios:
Cationic Lipid (30-70 mole %)
Non-cationic lipid (20-65 mole %) and
PEG-DMG (1-15 mole %).

In certain aspects of this embodiment, the non-cationic lipid is cholesterol. Exemplary LNPs may include cationic lipid/cholesterol/PEG-DMG at about the following molar ratios: 58/30/10.

In certain aspects of this embodiment, the non-cationic lipid is cholesterol and DSPC. Exemplary LNPs may include cationic lipid/cholesterol/DSPC/PEG-DMG at about the following molar ratios: 59/30/10/1; 58/30/10/2; 43/41/15/1; 42/41/15/2; 40/48/10/2; 39/41/19/1; 38/41/19/2; 34/41/24/1; and 33/41/24/2.

### Preparation of LNPs

LNPs can be formed, for example, by a rapid precipitation process which entails micro-mixing the lipid components dissolved in ethanol with an aqueous solution using a confined volume mixing apparatus such as a confined volume T-mixer, a multi-inlet vortex mixer (MIVM), or a microfluidics mixer device as described below. The lipid solution contains one or more cationic lipids, one or more noncationic lipids (e.g., DSPC), PEG-DMG, and optionally cholesterol, at specific molar ratios in ethanol. The aqueous solution consists of a sodium citrate or sodium acetate buffered salt solution with pH in the range of 2-6, preferably 3.5-5.5. The two solutions are heated to a temperature in the range of 25° C.-45° C., preferably 30° C.-40° C., and then mixed in a confined volume mixer thereby instantly forming the LNP. When a confined volume T-mixer is used, the T-mixer has an internal diameter (ID) range from 0.25 to 1.0 mm. The alcohol and aqueous solutions are delivered to the inlet of the T-mixer using programmable syringe pumps, and with a total flow rate from 10-600 mL/minute. The alcohol and aqueous solutions are combined in the confined-volume mixer with a ratio in the range of 1:1 to 1:3 vol:vol, but targeting 1:1.1 to 1:2.3. The combination of ethanol volume fraction, reagent solution flow rates and t-mixer tubing ID utilized at this mixing stage has the effect of controlling the particle size of the LNPs between 30 and 300 nm. The resulting LNP suspension is twice diluted into higher pH buffers in the range of 6-8 in a sequential, multi-stage in-line mixing process. For the first dilution, the LNP suspension is mixed with a buffered solution at a higher pH (pH 6-7.5) with a mixing ratio in the range of 1:1 to 1:3 vol:vol, but targeting 1:2 vol:vol. This buffered solution is at a temperature in the range of 15-40° C., targeting 30-40° C. The resulting LNP suspension is further mixed with a buffered solution at a higher pH, e.g., 6-8 and with a mixing ratio in the range of 1:1 to 1:3 vol:vol, but targeting 1:2 vol:vol. This later buffered solution is at a temperature in the range of 15-40° C., targeting 16-25° C. The mixed LNPs are held from 30 minutes to 2 hours prior to an anion exchange filtration step. The temperature during incubation period is in the range of 15-40° C., targeting 30-40° C. After incubation, the LNP suspension is filtered through a 0.8 μm filter containing an anion exchange separation step. This process uses tubing IDs ranging from 1 mm ID to 5 mm ID and a flow rate from 10 to 2000 mL/minute. The LNPs are concentrated and diafiltered via an ultrafiltration process where the alcohol is removed and the buffer is exchanged for the final buffer solution such as phosphate buffered saline or a buffer system suitable for cryopreservation (for example containing sucrose, trehalose or combinations thereof). The ultrafiltration process uses a tangential flow filtration format (TFF). This process uses a membrane nominal molecular weight cutoff range from 30-500 KD, targeting 100 KD. The membrane format can be hollow fiber or flat sheet cassette. The TFF processes with the proper molecular weight cutoff retains the LNP in the retentate and the filtrate or permeate contains the alcohol and final buffer wastes. The TFF process is a multiple step process with an initial concentration to a lipid concentration of 20-30 mg/mL. Following concentration, the LNP suspension is diafiltered against the final buffer (for example, phosphate buffered saline (PBS) with pH 7-8, 10 mM Tris, 140 mM NaCl with pH 7-8, or 10 mM Tris, 70 mM NaCl, 5 wt % sucrose, with pH 7-8) for 5-20 volumes to remove the alcohol and perform buffer exchange. The material is then concentrated an additional 1-3 fold via ultrafiltration. The final steps of the LNP manufacturing process are to sterile filter the concentrated LNP solution into a suitable container under aseptic conditions. Sterile filtration is accomplished by passing the LNP solution through a pre-filter (Acropak 500 PES 0.45/0.8 μm capsule) and a bioburden reduction filter (Acropak 500 PES 0.2/0.8 μm capsule). Following filtration, the vialed LNP product is stored under suitable storage conditions (2° C.-8° C., or −20° C. if frozen formulation).

In some embodiments, the LNPs of the compositions provided herein have a mean geometric diameter that is less than 1000 nm. In some embodiments, the LNPs have mean geometric diameter that is greater than 50 nm but less than 500 nm. In some embodiments, the mean geometric diameter of a population of LNPs is about 60 nm, 75 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, or 475 nm. In some embodiments, the mean geometric diameter is between 100-400 nm, 100-300 nm, 100-250 nm, or 100-200 nm. In some embodiments, the mean geometric diameter is between 60-400 nm, 60-350 nm, 60-300 nm, 60-250 nm, or 60-200 nm. In some embodiments, the mean geometric diameter is between 75-250 nm. In some embodiments, 30%, 40%, 50%, 60%, 70%, 80%, 90%, or more of the LNPs of a population of LNPs have a diameter that is less than 500 nm. In some embodiments, 10%, 20%, 30%, 40%, 50%, 60%, 70%, 80%, 90%, or more of the LNPs of a population of LNPs have a diameter that is greater than 50 nm but less than 500 nm. In some embodiments, 10%, 20%, 30%, 40%, 50%, 60%, 70%, 80%, 90%, or more of the LNPs of a population of LNPs have a diameter of about 60 nm, 75 nm, 100 nm, 125 nm, 150 nm, 175 nm, 200 nm, 225 nm, 250 nm, 275 nm, 300 nm, 325 nm, 350 nm, 375 nm, 400 nm, 425 nm, 450 nm, or 475 nm. In some embodiments, 10%, 20%, 30%, 40%, 50%, 60%, 70%, 80%, 90%, or more of the LNPs of a population of LNPs have a diameter that is between 100-400 nm, 100-300 nm, 100-250 nm, or 100-200 nm. In some embodiments, 10%, 20%, 30%, 40%, 50%, 60%, 70%, 80%, 90%, or more of the LNPs of a population of LNPs have a diameter that is between 60-400 nm, 60-350 nm, 60-300 nm, 60-250 nm, or 60-200 nm.

In a particular embodiment, the size of the LNPs ranges between about 1 and 1000 nm, preferably between about 10 and 500 nm, more preferably between about 100 to 300 nm, and preferably 100 nm.

### Therapeutic and Prophylactic Compositions

Provided herein are vaccine compositions (e.g., pharmaceutical compositions), methods, kits, and reagents for the prevention, treatment, and/or diagnosis of norovirus in humans and other mammals. Norovirus vaccines can be used as therapeutic or prophylactic agents. They may be used in medicine to prevent and/or treat infectious disease. In some embodiments, vaccines in accordance with the present disclosure may be used for treatment of norovirus.

Provided herein are pharmaceutical compositions including norovirus vaccines optionally in combination with one or more pharmaceutically acceptable excipients.

Norovirus vaccines may be formulated or administered alone or in conjunction with one or more other components. For instance, norovirus vaccines (vaccine compositions) may comprise other components including, but not limited to, adjuvants.

In some embodiments of the invention provided herein, norovirus vaccines do not include an adjuvant (i.e., they are adjuvant free). In other embodiments, norovirus vaccines include lipid or polymer-based nanoparticles. In certain embodiments, any of the lipid nanoparticles described herein may act as an adjuvant when combined with norovirus vaccines.

Aluminium has long been shown to stimulate the immune response against co-administered antigens, primarily by stimulating a TH2 response. It is preferred that the aluminium adjuvant of the compositions provided herein is not in the form of an aluminium precipitate. Aluminium-precipitated vaccines may increase the immune response to a target antigen, but have been shown to be highly heterogeneous preparations and have had inconsistent results (see Lindblad E. B. Immunology and Cell Biology 82:497-505 (2004)). Aluminium-adsorbed vaccines, in contrast, can be preformed in a standardized manner, which is an essential characteristic of vaccine preparations for administration into humans. Moreover, it is thought that physical adsorption of a desired antigen onto the aluminium adjuvant has an important role in adjuvant function, perhaps in part by allowing a slower clearing from the injection site or by allowing a more efficient uptake of antigen by antigen presenting cells.

The aluminium adjuvant of the present invention may be in the form of aluminium hydroxide (Al(OH)₃), aluminium phosphate (AIPO4), aluminium hydroxyphosphate, amorphous aluminium hydroxyphosphate sulfate (AAHS), or so-called “alum” (KA1(SO₄)-12H₂O). See Klein et al., Analysis of aluminium hydroxyphosphate vaccine adjuvants by (27)A1 MAS NMR., J. Pharm. Sci. 89 (3): 311-21 (2000).

In some embodiments of the invention provided herein, the aluminium adjuvant is aluminium hydroxyphosphate or AAHS. The ratio of phosphate to aluminium in the aluminium adjuvant can range from 0 to 1.3. In some embodiments of this aspect of the invention, the phosphate to aluminium ratio is within the range of 0.1 to 0.70. In certain embodiments, the phosphate to aluminium ratio is within the range of 0.2 to 0.50. APA is an aqueous suspension of aluminum hydroxyphosphate. APA is manufactured by blending aluminum chloride and sodium phosphate in a 1:1 volumetric ratio to precipitate aluminum hydroxyphosphate. After the blending process, the material is size-reduced with a high-shear mixer to achieve a target aggregate particle size in the range of 2-8 μm. The product is then diafiltered against physiological saline and steam sterilized. Sec, e.g., International Patent Application Publication No. WO2013/078102.

In some embodiments of the invention, the aluminium adjuvant is in the form of AAHS (referred to interchangeably herein as Merck aluminium adjuvant (MAA)). MAA carries zero charge at neutral pH, while AIOH carries a net positive charge and AIPO4 typically carries a net negative charge at neutral pH.

One of skill in the art will be able to determine an optimal dosage of aluminium adjuvant that is both safe and effective at increasing the immune response to the targeted antigenic polypeptides. For a discussion of the safety profile of aluminium, as well as amounts of aluminium included in FDA-licensed vaccines, see Baylor et al., Vaccine 20: S18-S23 (2002). Generally, an effective and safe dose of aluminium adjuvant varies from 150 to 600 μg/dose (300 to 1200 μg/mL concentration). In specific embodiments of the formulations and compositions of the present invention, there is between 200 and 300 μg aluminium adjuvant per dose of vaccine. In alternative embodiments of the formulations and compositions of the present invention, there is between 300 and 500 μg aluminium adjuvant per dose of vaccine.

### Norovirus Vaccine Formulations and Methods of Use

Norovirus vaccines of the present disclosure may be formulated or administered in combination with one or more pharmaceutically-acceptable excipients. In some embodiments, vaccine compositions comprise at least one additional active substances, such as, for example, a therapeutically-active substance, a prophylactically-active substance, or a combination of both. Vaccine compositions may be sterile, pyrogen-free, or both sterile and pyrogen-free. General considerations in the formulation and/or manufacture of pharmaceutical agents, such as vaccine compositions, may be found, for example, in Remington: The Science and Practice of Pharmacy 21st ed., Lippincott Williams & Wilkins, 2005.

Formulations of the norovirus vaccine compositions described herein may be prepared by any method known or hereafter developed in the art of pharmacology. In general, such preparatory methods include the step of bringing the active ingredient (e.g., one or more polynucleotides) into association with an excipient and/or one or more other accessory ingredients, and then, if necessary and/or desirable, dividing, shaping, and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, or at least 80% (w/w) active ingredient.

Some aspects of the present disclosure provide formulations of the norovirus vaccine, wherein the vaccine is formulated in an effective amount to produce an antigen specific immune response in a subject (e.g., production of antibodies specific to a norovirus antigenic polypeptide). An “effective amount” of a norovirus RNA vaccine is provided based, at least in part, on the target tissue, target cell type, means of administration, physical characteristics of the polynucleotide (e.g., size, and extent of modified nucleosides) and other components of the norovirus RNA vaccine, and other determinants. In general, an effective amount of the norovirus RNA vaccine composition provides an induced or boosted immune response as a function of antigen production in the cell, preferably more efficient than a composition containing a corresponding unmodified polynucleotide encoding the same antigen or a peptide antigen. Increased antigen production may be demonstrated by increased cell transfection (the percentage of cells transfected with the RNA vaccine), increased protein translation from the polynucleotide, decreased nucleic acid degradation (as demonstrated, for example, by increased duration of protein translation from a modified polynucleotide), or altered antigen specific immune response of the host cell.

In some embodiments, the antigen-specific immune response is characterized by measuring an anti-norovirus antigenic polypeptide antibody titer produced in a subject administered a norovirus vaccine as provided herein. An antibody titer is a measurement of the amount of antibodies within a subject, for example, antibodies that are specific to a particular antigen (e.g., a norovirus antigenic polypeptide, such as a VP1 or VP2 polypeptide) or epitope of an antigen. Antibody titer is typically expressed as the inverse of the greatest dilution that provides a positive result. Enzyme-linked immunosorbent assay (ELISA) is a common assay for determining antibody titers, for example.

In some embodiments, an antibody titer is used to assess whether a subject has had an infection or to determine whether immunizations are required. In some embodiments, an antibody titer is used to determine the strength of an autoimmune response, to determine whether a booster immunization is needed, to determine whether a previous vaccine was effective, and to identify any recent or prior infections. In accordance with the present disclosure, an antibody titer may be used to determine the strength of an immune response induced in a subject by the norovirus vaccine.

### Modes of Vaccine Administration

Norovirus vaccines may be administered prophylactically or therapeutically as part of an active immunization scheme to healthy individuals or early in infection during the incubation phase or during active infection after onset of symptoms. A prophylactically effective dose is a therapeutically effective dose that prevents infection with the virus at a clinically acceptable level. In some embodiments the therapeutically effective dose is a dose listed in a package insert for the vaccine. In some embodiments, the amount of vaccine of the present disclosure provided to a cell, a tissue, or a subject may be an amount effective for immune prophylaxis.

Norovirus vaccines may be formulated for administration by any route which results in a therapeutically effective outcome. These include, but are not limited, to intradermal, intramuscular, intranasal, intratracheal, and/or subcutaneous administration. In some embodiments, norovirus vaccines are administered intramuscularly.

The present disclosure provides methods comprising administering vaccines to a subject in need thereof. The exact amount required will vary from subject to subject, depending on the species, age, and general condition of the subject, the severity of the disease, the particular composition, its mode of administration, its mode of activity, and the like.

Norovirus vaccine compositions are typically formulated in dosage unit form for ease of administration and uniformity of dosage. It will be understood, however, that the total daily usage of vaccine compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

Norovirus vaccines may be administrated with other prophylactic or therapeutic compounds. As a non-limiting example, a prophylactic or therapeutic compound may be an adjuvant or a booster. As used herein, when referring to a prophylactic composition, such as a vaccine, the term “booster” refers to an extra administration of the prophylactic (vaccine) composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years, or more than 99 years. In some embodiments, the time of administration between the initial administration of the prophylactic composition and the booster may be, but is not limited to, 1 week, 2 weeks, 3 weeks, 1 month, 2 months, 3 months, 6 months, or 1 year.

Norovirus vaccines may be utilized in various settings depending on the prevalence of the infection or the degree or level of unmet medical need. As a non-limiting example, the norovirus vaccines may be utilized to treat and/or prevent a variety of noroviruses. Vaccines have superior properties in that they produce much larger antibody titers and produce responses earlier than commercially available anti-viral agents and compositions.

### Methods of Treatment

Provided herein are compositions (e.g., pharmaceutical compositions), methods, kits, and reagents for prevention and/or treatment of norovirus in humans and other mammals. Norovirus vaccines can be used as therapeutic or prophylactic agents. They may be used in medicine to prevent and/or treat infectious disease. In exemplary aspects, the norovirus vaccines of the present disclosure are used to provide prophylactic protection from norovirus. Prophylactic protection from norovirus can be achieved following administration of a norovirus vaccine of the present disclosure. Vaccines can be administered once, twice, three times, four times, or more. It is possible, although less desirable, to administer the vaccine to an infected individual to achieve a therapeutic response. Dosing may need to be adjusted accordingly.

In some embodiments, the norovirus vaccines of the present disclosure can be used as a method of preventing a norovirus infection in a subject, the method comprising administering to said subject at least one norovirus vaccine as provided herein. In some embodiments, the norovirus vaccines of the present disclosure can be used as a method of inhibiting a primary norovirus infection in a subject, the method comprising administering to said subject at least one norovirus vaccine as provided herein. In some embodiments, the norovirus vaccines of the present disclosure can be used as a method of treating a norovirus infection in a subject, the method comprising administering to said subject at least one norovirus vaccine as provided herein. In some embodiments, the norovirus vaccines of the present disclosure can be used as a method of reducing an incidence of norovirus infection in a subject, the method comprising administering to said subject at least one norovirus vaccine as provided herein. In some embodiments, the norovirus vaccines of the present disclosure can be used as a method of inhibiting spread of norovirus from a first subject infected with norovirus to a second subject not infected with norovirus, the method comprising administering to at least one of said first subject and said second subject at least one norovirus vaccine as provided herein.

A method of eliciting an immune response in a subject against a norovirus is provided in aspects of the invention. The method involves administering to the subject a norovirus vaccine described herein, thereby inducing in the subject an immune response specific to norovirus antigenic polypeptide or an immunogenic fragment thereof.

### EXAMPLES

The following examples are meant to be illustrative and should not be construed as further limiting. The contents of the figures and all references, patents, and published patent applications cited throughout this application are expressly incorporated herein by reference.

### Example 1: Preparation of mRNAs

DNA sequences encoding norovirus antigenic VP1 proteins were prepared and used for subsequent RNA in vitro transcription reactions. VP1 protein sequences were retrieved from public databases (GenBank, INSDC), and a single consensus sequence was selected for each genotype (i.e., GenBank ID QBW96040 for GII.4, GenBank ID NP_056821 for GI.1, GenBank ID AWT08315 for GII.6, GenBank ID for YP_009518839 GII.2, and GenBank for ID QJF54133 GII.3) based on the most common recent sequence. The selected DNA sequences were modified by introducing a codon modified sequence or GC-optimized sequence for stabilization. DNA sequences were changed from the native sequence as follows. Silent mutations were introduced to maximize codon adaptation index (CAI). See Sharp P M, Li W H. The codon Adaptation Index-a measure of directional synonymous codon usage bias, and its potential applications. Nucleic Acids Res. 1987 Feb. 11; 15 (3): 1281-95. Runs of five or more of the same nucleotide were broken using the second most frequent codon. In addition, DNA 30-mers with G/C content above 80% were mutated to reduce G/C content to 80% or below, using the second most frequent codon of Pro (CCC->CCA), Gly (GGC->GGA) and/or Ala (GCC->GCA). For example, using the short amino acid sequence “Proline-Proline,” as found, e.g., in the GII.3 DNA: (1) the native sequence CCC-CCT was first mutated to CCC-CCC to optimize CAI; and then (2) further modified to the second most frequent codon for Proline (i.e., CCA) to avoid homopolymer runs. The codon optimized DNA sequences are shown as SEQ ID NOs: 9-13 of Table 1 below.

TABLE 1
Sequences

| SEQ | | |
|---|---|---|
| ID | | |
| NO: | Description | Sequence |
| 1 | GII.4 | AUGAAGAUGGCCAGCAGCGACGCCAACCCAAGCGACG |
| | Organism: | GCAGCGCAGCCAACCUGGUGCCCGAGGUGAACAACGA |
| | Synthetic | GGUGAUGGCCCUGGAGCCAGUGGUGGGAGCAGCCAU |
| | construct | CGCAGCCCCAGUGGCAGGCCAGCAGAACGUGAUCGAC |
| | Mol. Type: | CCAUGGAUCCGGAACAACUUCGUGCAGGCCCCAGGAG |
| | Other RNA | GCGAGUUCACCGUGAGCCCACGGAACGCCCCAGGCGA |
| | | GAUCCUGUGGAGCGCACCACUGGGCCCAGACCUGAAC |
| | | CCAUACCUGAGCCACCUGGCCCGGAUGUACAACGGCU |
| | | ACGCCGGCGGCUUCGAGGUGCAGGUGAUCCUGGCCGG |
| | | CAACGCCUUCACCGCAGGCAAGAUCAUCUUCGCCGCC |
| | | GUGCCACCCAACUUCCCAACCGAGGGCCUGAGCCCAA |
| | | GCCAGGUGACCAUGUUCCCACACAUCAUCGUGGACGU |
| | | GAGGCAGCUGGAGCCCGUGCUGAUCCCACUGCCCGAC |
| | | GUGCGGAACAACUUCUACCACUACAACCAGAGCAACG |
| | | ACAGCACCAUCAAGCUGAUCGCCAUGCUGUACACCCC |
| | | ACUGCGGGCCAACAACGCCGGCGACGACGUGUUCACC |
| | | GUGAGCUGCAGGGUGCUGACCAGGCCCAGCCCAGACU |
| | | UCGACUUCAUCUUCCUGGUGCCACCCACCGUGGAGAG |
| | | CCGGACCAAGCCCUUCAGCGUGCCCGUGCUGACCGUG |
| | | GAGGAGAUGACCAACAGCCGGUUCCCAAUCCCACUGG |
| | | AGAAGCUGUUCACCGGACCAAGCAGCGCCUUCGUGGU |
| | | GCAGCCACAGAACGGCCGGUGCACCACCGACGGAGUG |
| | | CUGCUGGGCACCACCCAGCUGAGCCCAGUGAACAUCU |
| | | GCACCUUCCGGGGCGACGUGACCCACAUCACCGGCAG |
| | | CCGGAACUACACCAUGAACCUGGCCAGCCAGAACUGG |
| | | AACAACUACGACCCAACCGAGGAGAUCCCAGCCCCAC |
| | | UGGGCACCCCAGACUUCGUGGGCAAGAUCCAGGGCAU |
| | | GCUGACCCAGACCACCAGAACCGACGGCAGCACCAGA |
| | | GGCCACAAGGCCACCGUGUACACCGGCAGCGCCGACU |
| | | UCGCCCCAAAGCUGGGCCGGGUGCAGUUCGAGACCGA |
| | | CACCGACCACGACUUCGAGGCCAACCAGAACACCAAG |
| | | UUCACCCCAGUGGGCGUGAUCCAGGACGGCAGCACCA |
| | | CCCACCGGAACGAGCCACAGCAGUGGGUGCUGCCCAG |
| | | CUACAGCGGCAGGAACACCCACAACGUGCACCUGGCA |
| | | CCAGCCGUGGCACCAACCUUCCCAGGCGAGCAGCUGC |
| | | UGUUCUUCCGGAGCACCAUGCCAGGCUGCAGCGGCUA |
| | | CCCAAACAUGGACCUGGACUGCCUGCUGCCACAGGAG |
| | | UGGGUGCAGUACUUCUACCAGGAGGCAGCCCCAGCAC |
| | | AGAGCGACGUGGCCCUGCUGCGGUUCGUGAACCCAGA |
| | | CACCGGACGGGUGCUGUUCGAGUGCAAGCUGCACAA |
| | | GAGCGGCUACGUGACCGUGGCCCACACCGGACAGCAC |
| | | GACCUGGUGAUCCCACCCAACGGCUACUUCCGGUUCG |
| | | ACAGCUGGGUGAACCAGUUCUACACCCUGGCCCCAAU |
| | | GGGCAACGGAACCGGACGGAGGAGGGUGGUG |
| 2 | GI.1 | AUGAUGAUGGCCAGCAAGGACGCCACCAGCAGCGUG |
| | Organism: | GACGGAGCCAGCGGCGCAGGACAGCUGGUGCCCGAGG |
| | Synthetic | UGAACGCCAGCGACCCACUGGCCAUGGACCCAGUGGC |
| | construct | CGGAAGCAGCACCGCAGUGGCCACCGCAGGCCAGGUG |
| | Mol. Type: | AACCCAAUCGACCCAUGGAUCAUCAACAACUUCGUGC |
| | Other RNA | AGGCCCCACAGGGCGAGUUCACCAUCAGCCCAAACAA |
| | | CACCCCAGGCGACGUGCUGUUCGACCUGAGCCUGGGC |
| | | CCACACCUGAACCCAUUCCUGCUGCACCUGAGCCAGA |
| | | UGUACAACGGCUGGGUGGGCAACAUGCGGGUGCGGA |
| | | UCAUGCUGGCCGGCAACGCCUUCACCGCCGGCAAGAU |
| | | CAUCGUGAGCUGCAUCCCACCCGGCUUCGGCAGCCAC |
| | | AACCUGACCAUCGCCCAGGCCACCCUGUUCCCACACG |
| | | UGAUCGCCGACGUGCGGACCCUGGACCCAAUCGAGGU |
| | | GCCACUGGAGGACGUGCGGAACGUGCUGUUCCACAAC |
| | | AACGACCGGAACCAGCAGACCAUGCGGCUGGUGUGCA |
| | | UGCUGUACACCCCACUGCGGACCGGAGGCGGAACCGG |
| | | AGACAGCUUCGUGGUGGCCGGCCGGGUGAUGACCUG |
| | | CCCAAGCCCAGACUUCAACUUCCUGUUCCUGGUGCCA |
| | | CCCACCGUGGAGCAGAAGACCCGGCCCUUCACCCUGC |
| | | CCAACCUGCCACUGAGCAGCCUGAGCAACAGCCGGGC |
| | | CCCACUGCCCAUCAGCAGCAUGGGCAUCAGCCCAGAC |
| | | AACGUGCAGAGCGUGCAGUUCCAGAACGGCCGGUGC |
| | | ACCCUGGACGGACGGCUGGUGGGCACCACCCCAGUGA |
| | | GCCUGAGCCACGUGGCCAAGAUCCGGGGCACCAGCAA |
| | | CGGCACCGUGAUCAACCUGACCGAGCUGGACGGCACC |
| | | CCAUUCCACCCAUUCGAGGGCCCAGCCCCAAUCGGCU |
| | | UCCCAGACCUGGGCGGCUGCGACUGGCACAUCAACAU |
| | | GACCCAGUUCGGCCACAGCAGCCAGACCCAGUACGAC |
| | | GUGGACACCACCCCAGACACCUUCGUGCCACACCUGG |
| | | GCAGCAUCCAGGCCAACGGCAUCGGCAGCGGCAACUA |
| | | CGUGGGCGUGCUGAGCUGGAUCAGCCCACCCAGCCAC |
| | | CCAAGCGGCAGCCAGGUGGACCUGUGGAAGAUCCCAA |
| | | ACUACGGCAGCAGCAUCACCGAGGCCACCCACCUGGC |
| | | CCCAAGCGUGUACCCACCCGGCUUCGGCGAGGUGCUG |
| | | GUGUUCUUCAUGAGCAAGAUGCCCGGCCCAGGCGCCU |
| | | ACAACCUGCCCUGCCUGCUGCCACAGGAGUACAUCAG |
| | | CCACCUGGCCAGCGAGCAGGCCCCAACCGUGGGCGAG |
| | | GCAGCCCUGCUGCACUACGUGGACCCAGACACCGGCC |
| | | GGAACCUGGGCGAGUUCAAGGCCUACCCAGACGGCUU |
| | | CCUGACCUGCGUGCCCAACGGAGCCAGCAGCGGCCCA |
| | | CAGCAGCUGCCCAUCAACGGCGUGUUCGUGUUCGUGA |
| | | GCUGGGUGAGCCGGUUCUACCAGCUGAAGCCCGUGG |
| | | GAACCGCCAGCAGCGCCAGAGGACGGCUGGGACUGAG |
| | | GCGG |
| 3 | GII.6 | AUGAAGAUGGCCAGCAACGACGCCGCCCCAAGCAACG |
| | Organism: | ACGGAGCCGCCAACCUGGUGCCCGAGGCCACCAACGA |
| | Synthetic | GGUGAUGGCCCUGGAGCCCGUGGUGGGAGCCAGCAU |
| | construct | CGCAGCCCCAGUGGUGGGCCAGCAGAACAUCAUCGAC |
| | Mol. Type: | CCAUGGAUCCGGGAGAACUUCGUGCAGGCCCCACAGG |
| | Other RNA | GCGAGUUCACCGUGAGCCCACGGAACAGCCCAGGCGA |
| | | GAUGCUGCUGAACCUGGAGCUGGGCCCAGAGCUGAA |
| | | CCCAUACCUGGGCCACCUGAGCCGGAUGUACAACGGC |
| | | UACGCCGGCGGCAUGCAGGUGCAGGUGGUGCUGGCC |
| | | GGCAACGCCUUCACCGCCGGCAAGAUCAUCUUCGCCG |
| | | CCGUGCCACCACACUUCCCAGUGGAGAACAUCAGCGC |
| | | CGCCCAGAUCACCAUGUGCCCACACGUGAUCGUGGAC |
| | | GUGCGGCAGCUGGAGCCCGUGCUGCUGCCACUGCCCG |
| | | ACAUCCGGAACCGGUUCUUCCACUACAACCAGGAGAA |
| | | CACCCCACGGAUGCGGCUGGUGGCCAUGCUGUACACC |
| | | CCACUGCGGGCCAACAGCGGCGAGGACGUGUUCACCG |
| | | UGAGCUGCCGGGUGCUGACCAGGCCAGCCCCAGACUU |
| | | CGAGUUCACCUUCCUGGUGCCACCCACCGUGGAGAGC |
| | | AAGACCAAGCCCUUCACCCUGCCCAUCCUGACCCUGG |
| | | GCGAGCUGAGCAACAGCCGGUUCCCAGCCCCAAUCGA |
| | | CAUGCUGUACACCGACCCAAACGAGGCCAUCGUGGUG |
| | | CAGCCACAGAACGGCCGGUGCACCCUGGACGGCACCC |
| | | UGCAGGGCACCACCCAGCUGGUGCCCACCCAGAUCUG |
| | | CAGCUUCCGGGGCACCCUGAUCAGCCAGACCAGCCGG |
| | | AGCGCAGACAGCACCGACAGCGCCCCACGGGUGCGGA |
| | | ACCACCCACUGCACGUGCAGCUGAAGAACCUGGACGG |
| | | CACCCCAUACGACCCAACCGACGAGGUGCCCGCAGUG |
| | | CUGGGCGCCAUCGACUUCAAGGGCACCGUGUUCGGCA |
| | | UCGCCAGCCAGCGGAACACCACCGGCAGCAGCAUCGG |
| | | AGCCACCCGGGCACACGAGGUGCACAUCGACACCACC |
| | | AACCCACGGUACACCCCAAAGCUGGGCAGCGUGCUGA |
| | | UGUACAGCGAGAGCAACGACUUCGACGACGGCCAGCC |
| | | CACCCGGUUCACCCCAAUCGGCAUGGGCGCCGACGAC |
| | | UGGCGGCAGUGGGAGCUGCCCGAGUACAGCGGCCACC |
| | | UGACCCUGAACAUGAACCUGGCCCCAGCCGUGGCACC |
| | | AGCCUUCCCAGGCGAGCGGAUCCUGUUCUUCCGGAGC |
| | | GUGGUGCCCAGCGCAGGAGGCUACGGCAGCGGCCACA |
| | | UCGACUGCCUGAUCCCACAGGAGUGGGUGCAGCACUU |
| | | CUACCAGGAGGCCGCACCAAGCCAGAGCGCCGUGGCC |
| | | CUGAUCCGGUACGUGAACCCAGACACCGGCCGGAACA |
| | | UCUUCGAGGCCAAGCUGCACCGGGAGGGCUUCAUCAC |
| | | CGUGGCCAACAGCGGCAACAACCCAAUCGUGGUGCCA |
| | | CCCAACGGCUACUUCCGGUUCGAGGCCUGGGUGAACC |
| | | AGUUCUACACCCUGACCCCAAUGGGCACCGGACAGGG |
| | | ACGGAGGCGGGUGCAG |
| 4 | GII.2 | AUGAAGAUGGCCAGCAACGACGCCGCACCAAGCACCG |
| | Organism: | ACGGAGCCGCAGGACUGGUGCCCGAGAGCAACAACGA |
| | Synthetic | GGUGAUGGCCCUGGAGCCCGUGGCAGGAGCCGCACUG |
| | construct | GCAGCACCAGUGACCGGCCAGACCAACAUCAUCGACC |
| | Mol. Type: | CAUGGAUCCGGGCCAACUUCGUGCAGGCCCCAAACGG |
| | Other RNA | CGAGUUCACCGUGAGCCCACGGAACGCCCCAGGCGAG |
| | | GUGCUGCUGAACCUGGAGCUGGGCCCAGAGCUGAACC |
| | | CAUACCUGGCCCACCUGGCCCGGAUGUACAACGGCUA |
| | | CGCCGGCGGCAUGGAGGUGCAGGUGAUGCUGGCCGG |
| | | CAACGCCUUCACCGCCGGCAAGCUGGUGUUCGCCGCC |
| | | GUGCCACCACACUUCCCAGUGGAGAACCUGAGCCCAC |
| | | AGCAGAUCACCAUGUUCCCACACGUGAUCAUCGACGU |
| | | GCGGACCCUGGAGCCCGUGCUGCUGCCACUGCCCGAC |
| | | GUGCGGAACAACUUCUUCCACUACAACCAGAAGGACG |
| | | ACCCAAAGAUGCGGAUCGUGGCCAUGCUGUACACCCC |
| | | ACUGCGGAGCAACGGCAGCGGCGACGACGUGUUCACC |
| | | GUGAGCUGCCGGGUGCUGACCAGGCCCAGCCCAGACU |
| | | UCGACUUCACCUACCUGGUGCCACCCACCGUGGAGAG |
| | | CAAGACCAAGCCCUUCACCCUGCCCAUCCUGACCCUG |
| | | GGCGAGCUGAGCAACAGCCGGUUCCCAGUGAGCAUCG |
| | | ACCAGAUGUACACCAGCCCAAACGAGAUCAUCAGCGU |
| | | GCAGUGCCAGAACGGCCGGUGCACCCUGGACGGCGAG |
| | | CUGCAGGGCACCACCCAGCUGCAGGUGAGCGGCAUCU |
| | | GCGCCUUCAAGGGCGAGGUGACCGCCCACCUGCACGA |
| | | CAACGACCACCUGUACAACGUGACCAUCACCAACCUG |
| | | AACGGCAGCCCAUUCGACCCAAGCGAGGACAUCCCAG |
| | | CCCCACUGGGCGUGCCCGACUUCCAGGGCCGGGUGUU |
| | | CGGCAUCAUCAGCCAGCGGGACAAGCACAACAGCCCA |
| | | GGCCACAACGAGCCCGCCAACCGGGGACACGACGCCG |
| | | UGGUGCCCACCUACACCGCCCAGUACACCCCAAAGCU |
| | | GGGCCAGAUCCAGAUCGGCACCUGGCAGACCGACGAC |
| | | CUGACCGUGAACCAGCCCGUGAAGUUCACCCCAGUGG |
| | | GCCUGAACGACACCGAGCACUUCAACCAGUGGGUGGU |
| | | GCCACGGUACGCCGGCGCCCUGAACCUGAACACCAAC |
| | | CUGGCCCCAAGCGUGGCCCCAGUGUUCCCAGGCGAGC |
| | | GGCUGCUGUUCUUCCGGAGCUACAUCCCACUGAAGGG |
| | | CGGCUACGGCAACCCAGCCAUCGACUGCCUGCUGCCA |
| | | CAGGAGUGGGUGCAGCACUUCUACCAGGAGGCCGCCC |
| | | CAAGCAUGAGCGAGGUGGCCCUGGUGCGGUACAUCA |
| | | ACCCAGACACCGGCCGGGCCCUGUUCGAGGCCAAGCU |
| | | GCACCGGGCCGGCUUCAUGACCGUGAGCAGCAACACC |
| | | AGCGCCCCAGUGGUGGUGCCCGCCAACGGCUACUUCC |
| | | GGUUCGACAGCUGGGUGAACCAGUUCUACAGCCUGG |
| | | CCCCAAUGGGCACCGGCAACGGACGGAGGCGGGUGCA |
| | | G |
| 5 | GII.3 | AUGAAGAUGGCCAGCAACGACGCCACCCCAAGCAACG |
| | Organism: | ACGGAGCCGCAGGCCUGGUGCCCGAGAUCAACAACGA |
| | Synthetic | GGCCAUGGCCCUGGAGCCCGUGGCAGGAGCAGCCAUC |
| | construct | GCCGCACCACUGACCGGCCAGCAGAACAUCAUCGACC |
| | Mol. Type: | CAUGGAUCAUGAACAACUUCGUGCAGGCCCCAGGCGG |
| | Other RNA | CGAGUUCACCGUGAGCCCACGGAACAGCCCAGGCGAG |
| | | GUGCUGCUGAACCUGGAGCUGGGCCCAGAGAUCAACC |
| | | CAUACCUGGCCCACCUGGCCCGGAUGUACAACGGCUA |
| | | CGCCGGCGGCUUCGAGGUGCAGGUGGUGCUGGCCGGC |
| | | AACGCCUUCACCGCCGGCAAGAUCAUCUUCGCCGCCA |
| | | UCCCACCCAACUUCCCAAUCGACAACCUGAGCGCCGC |
| | | CCAGAUCACCAUGUGCCCACACGUGAUCGUGGACGUG |
| | | CGGCAGCUGGAGCCCGUGAACCUGCCCAUGCCCGACG |
| | | UGCGGAACAACUUCUUCCACUACAACCAGGGAAGCGA |
| | | CAGCCGGCUGCGGCUGGUGGCCAUGCUGUACACCCCA |
| | | CUGCGGGCCAACAACAGCGGCGACGACGUGUUCACCG |
| | | UGAGCUGCCGGGUGCUGACCAGGCCCAGCCCAGAGUU |
| | | CAGCUUCAACUUCCUGGUGCCACCCACCGUGGAGAGC |
| | | AAGACCAAGCCCUUCACCCUGCCCAUCCUGACCAUCA |
| | | GCGAGAUGAGCAACAGCCGGUUCCCAGUGCCCAUCGA |
| | | CAGCCUGCACACCAGCCCAACCGAGAACAUCGUGGUG |
| | | CAGUGCCAGAACGGCCGGGUGACCCUGGACGGCGAGC |
| | | UGAUGGGCACCACCCAGCUGCUGCCCAGCCAGAUCUG |
| | | CGCCUUCCGGGGAGUGCUGACCCGGAGCACCAGCCGG |
| | | GCAAGCGACCAGGCAGACACCGCCACCCCACGGCUGU |
| | | UCAACUACUACUGGCACAUCCAGCUGGACAACCUGAA |
| | | CGGCACCCCAUACGACCCAGCCGAGGACAUCCCAGGC |
| | | CCACUGGGCACCCCAGACUUCCGGGGCAAGGUGUUCG |
| | | GCGUGGCCAGCCAGCGGAACCCAGACAGCACCACCCG |
| | | GGCCCACGAGGCCAAGGUGGACACCACCGCCGGCCGG |
| | | UUCACCCCAAAGCUGGGCAGCCUGGAGAUCAGCACCG |
| | | AGAGCGACGACUUCGACCAGAACCAGCCCACCCGGUU |
| | | CACCCCAGUGGGCAUCGGCGUGGACAACGAGGCCGAC |
| | | UUCCAGCAGUGGAGCCUGCCCGACUACAGCGGCCAGU |
| | | UCACCCACAACAUGAACCUGGCCCCAGCCGUGGCCCC |
| | | AAACUUCCCAGGCGAGCAGCUGCUGUUCUUCCGGAGC |
| | | CAGCUGCCCAGCAGCGGAGGACGGAGCAACGGCAUCC |
| | | UGGACUGCCUGGUGCCACAGGAGUGGGUGCAGCACU |
| | | UCUACCAGGAGAGCGCACCAGCCCAGACCCAGGUGGC |
| | | CCUGGUGCGGUACGUGAACCCAGACACCGGCCGGGUG |
| | | CUGUUCGAGGCCAAGCUGCACAAGCUGGGCUUCAUG |
| | | ACCAUCGCCAAGAACGGCGACAGCCCAAUCACCGUGC |
| | | CACCCAACGGCUACUUCCGGUUCGAGAGCUGGGUGAA |
| | | CCCAUUCUACACCCUGGCCCCAAUGGGCACCGGCAAC |
| | | GGACGGAGGCGGGUGCAG |
| 6 | 5′ UTR | A(2′OMe)GGAAAUAAGAGAGAAAAGAAGAGUAAGAAG |
| | Organism: | AAAUUAAGAGCCACC |
| | Synthetic | |
| | construct | |
| | Mol. Type: | |
| | Other RNA | |
| 7 | 3′ UTR | GCGGCCGCUUAAUUAAGCUGCCUUCUGCGGGGCUUGC |
| | Organism: | CUUCUGGCCAUGCCCUUCUUCUCUCCCUUGCACCUGU |
| | Synthetic | ACCUCUUGGUCUUUGAAUAAAGCCUGAGUAGGAAGU |
| | construct | CUAGAAAAAAAAAAAAAAAAAAAAAAAAAAAAAAAA |
| | Mol. Type: | AAAAAAAAAAAAAAAAAAAAAAAAAAAAAAAAAAAA |
| | Other RNA | AAAAAAAAAAAA |
| 8 | 3′ UTR | GCUGCCUUCUGCGGGGCUUGCCUUCUGGCCAUGCCCU |
| | Organism: | UCUUCUCUCCCUUGCACCUGUACCUCUUGGUCUUUGA |
| | Synthetic | AUAAAGCCUGAGUAGGAAGAAAAAAAAAAAAAAAAA |
| | construct | AAAAAAAAAAAAAAAAAAAAAAAAAAAAAAAAAAAA |
| | Mol. Type: | AAAAAAAAAAAAAAAAAAAAAAAAAAA |
| | Other RNA | |
| 9 | GII.4 | ATGAAGATGGCCAGCAGCGACGCCAACCCAAGCGACG |
| | Organism: | GCAGCGCAGCCAACCTGGTGCCCGAGGTGAACAACGA |
| | Synthetic | GGTGATGGCCCTGGAGCCAGTGGTGGGAGCAGCCATC |
| | construct | GCAGCCCCAGTGGCAGGCCAGCAGAACGTGATCGACC |
| | Mol. Type: | CATGGATCCGGAACAACTTCGTGCAGGCCCCAGGAGG |
| | Other DNA | CGAGTTCACCGTGAGCCCACGGAACGCCCCAGGCGAG |
| | | ATCCTGTGGAGCGCACCACTGGGCCCAGACCTGAACCC |
| | | ATACCTGAGCCACCTGGCCCGGATGTACAACGGCTACG |
| | | CCGGCGGCTTCGAGGTGCAGGTGATCCTGGCCGGCAAC |
| | | GCCTTCACCGCAGGCAAGATCATCTTCGCCGCCGTGCC |
| | | ACCCAACTTCCCAACCGAGGGCCTGAGCCCAAGCCAG |
| | | GTGACCATGTTCCCACACATCATCGTGGACGTGAGGCA |
| | | GCTGGAGCCCGTGCTGATCCCACTGCCCGACGTGCGGA |
| | | ACAACTTCTACCACTACAACCAGAGCAACGACAGCAC |
| | | CATCAAGCTGATCGCCATGCTGTACACCCCACTGCGGG |
| | | CCAACAACGCCGGCGACGACGTGTTCACCGTGAGCTGC |
| | | AGGGTGCTGACCAGGCCCAGCCCAGACTTCGACTTCAT |
| | | CTTCCTGGTGCCACCCACCGTGGAGAGCCGGACCAAGC |
| | | CCTTCAGCGTGCCCGTGCTGACCGTGGAGGAGATGACC |
| | | AACAGCCGGTTCCCAATCCCACTGGAGAAGCTGTTCAC |
| | | CGGACCAAGCAGCGCCTTCGTGGTGCAGCCACAGAAC |
| | | GGCCGGTGCACCACCGACGGAGTGCTGCTGGGCACCA |
| | | CCCAGCTGAGCCCAGTGAACATCTGCACCTTCCGGGGC |
| | | GACGTGACCCACATCACCGGCAGCCGGAACTACACCA |
| | | TGAACCTGGCCAGCCAGAACTGGAACAACTACGACCC |
| | | AACCGAGGAGATCCCAGCCCCACTGGGCACCCCAGAC |
| | | TTCGTGGGCAAGATCCAGGGCATGCTGACCCAGACCAC |
| | | CAGAACCGACGGCAGCACCAGAGGCCACAAGGCCACC |
| | | GTGTACACCGGCAGCGCCGACTTCGCCCCAAAGCTGGG |
| | | CCGGGTGCAGTTCGAGACCGACACCGACCACGACTTCG |
| | | AGGCCAACCAGAACACCAAGTTCACCCCAGTGGGCGT |
| | | GATCCAGGACGGCAGCACCACCCACCGGAACGAGCCA |
| | | CAGCAGTGGGTGCTGCCCAGCTACAGCGGCAGGAACA |
| | | CCCACAACGTGCACCTGGCACCAGCCGTGGCACCAACC |
| | | TTCCCAGGCGAGCAGCTGCTGTTCTTCCGGAGCACCAT |
| | | GCCAGGCTGCAGCGGCTACCCAAACATGGACCTGGAC |
| | | TGCCTGCTGCCACAGGAGTGGGTGCAGTACTTCTACCA |
| | | GGAGGCAGCCCCAGCACAGAGCGACGTGGCCCTGCTG |
| | | CGGTTCGTGAACCCAGACACCGGACGGGTGCTGTTCGA |
| | | GTGCAAGCTGCACAAGAGCGGCTACGTGACCGTGGCC |
| | | CACACCGGACAGCACGACCTGGTGATCCCACCCAACG |
| | | GCTACTTCCGGTTCGACAGCTGGGTGAACCAGTTCTAC |
| | | ACCCTGGCCCCAATGGGCAACGGAACCGGACGGAGGA |
| | | GGGTGGTG |
| 10 | GI.1 | ATGATGATGGCCAGCAAGGACGCCACCAGCAGCGTGG |
| | Organism: | ACGGAGCCAGCGGCGCAGGACAGCTGGTGCCCGAGGT |
| | Synthetic | GAACGCCAGCGACCCACTGGCCATGGACCCAGTGGCC |
| | construct | GGAAGCAGCACCGCAGTGGCCACCGCAGGCCAGGTGA |
| | Mol. Type: | ACCCAATCGACCCATGGATCATCAACAACTTCGTGCAG |
| | Other DNA | GCCCCACAGGGCGAGTTCACCATCAGCCCAAACAACA |
| | | CCCCAGGCGACGTGCTGTTCGACCTGAGCCTGGGCCCA |
| | | CACCTGAACCCATTCCTGCTGCACCTGAGCCAGATGTA |
| | | CAACGGCTGGGTGGGCAACATGCGGGTGCGGATCATG |
| | | CTGGCCGGCAACGCCTTCACCGCCGGCAAGATCATCGT |
| | | GAGCTGCATCCCACCCGGCTTCGGCAGCCACAACCTGA |
| | | CCATCGCCCAGGCCACCCTGTTCCCACACGTGATCGCC |
| | | GACGTGCGGACCCTGGACCCAATCGAGGTGCCACTGG |
| | | AGGACGTGCGGAACGTGCTGTTCCACAACAACGACCG |
| | | GAACCAGCAGACCATGCGGCTGGTGTGCATGCTGTACA |
| | | CCCCACTGCGGACCGGAGGCGGAACCGGAGACAGCTT |
| | | CGTGGTGGCCGGCCGGGTGATGACCTGCCCAAGCCCA |
| | | GACTTCAACTTCCTGTTCCTGGTGCCACCCACCGTGGA |
| | | GCAGAAGACCCGGCCCTTCACCCTGCCCAACCTGCCAC |
| | | TGAGCAGCCTGAGCAACAGCCGGGCCCCACTGCCCATC |
| | | AGCAGCATGGGCATCAGCCCAGACAACGTGCAGAGCG |
| | | TGCAGTTCCAGAACGGCCGGTGCACCCTGGACGGACG |
| | | GCTGGTGGGCACCACCCCAGTGAGCCTGAGCCACGTG |
| | | GCCAAGATCCGGGGCACCAGCAACGGCACCGTGATCA |
| | | ACCTGACCGAGCTGGACGGCACCCCATTCCACCCATTC |
| | | GAGGGCCCAGCCCCAATCGGCTTCCCAGACCTGGGCG |
| | | GCTGCGACTGGCACATCAACATGACCCAGTTCGGCCAC |
| | | AGCAGCCAGACCCAGTACGACGTGGACACCACCCCAG |
| | | ACACCTTCGTGCCACACCTGGGCAGCATCCAGGCCAAC |
| | | GGCATCGGCAGCGGCAACTACGTGGGCGTGCTGAGCT |
| | | GGATCAGCCCACCCAGCCACCCAAGCGGCAGCCAGGT |
| | | GGACCTGTGGAAGATCCCAAACTACGGCAGCAGCATC |
| | | ACCGAGGCCACCCACCTGGCCCCAAGCGTGTACCCACC |
| | | CGGCTTCGGCGAGGTGCTGGTGTTCTTCATGAGCAAGA |
| | | TGCCCGGCCCAGGCGCCTACAACCTGCCCTGCCTGCTG |
| | | CCACAGGAGTACATCAGCCACCTGGCCAGCGAGCAGG |
| | | CCCCAACCGTGGGCGAGGCAGCCCTGCTGCACTACGTG |
| | | GACCCAGACACCGGCCGGAACCTGGGCGAGTTCAAGG |
| | | CCTACCCAGACGGCTTCCTGACCTGCGTGCCCAACGGA |
| | | GCCAGCAGCGGCCCACAGCAGCTGCCCATCAACGGCG |
| | | TGTTCGTGTTCGTGAGCTGGGTGAGCCGGTTCTACCAG |
| | | CTGAAGCCCGTGGGAACCGCCAGCAGCGCCAGAGGAC |
| | | GGCTGGGACTGAGGCGG |
| 11 | GII.6 | ATGAAGATGGCCAGCAACGACGCCGCCCCAAGCAACG |
| | Organism: | ACGGAGCCGCCAACCTGGTGCCCGAGGCCACCAACGA |
| | Synthetic | GGTGATGGCCCTGGAGCCCGTGGTGGGAGCCAGCATC |
| | construct | GCAGCCCCAGTGGTGGGCCAGCAGAACATCATCGACC |
| | Mol. Type: | CATGGATCCGGGAGAACTTCGTGCAGGCCCCACAGGG |
| | Other DNA | CGAGTTCACCGTGAGCCCACGGAACAGCCCAGGCGAG |
| | | ATGCTGCTGAACCTGGAGCTGGGCCCAGAGCTGAACCC |
| | | ATACCTGGGCCACCTGAGCCGGATGTACAACGGCTACG |
| | | CCGGCGGCATGCAGGTGCAGGTGGTGCTGGCCGGCAA |
| | | CGCCTTCACCGCCGGCAAGATCATCTTCGCCGCCGTGC |
| | | CACCACACTTCCCAGTGGAGAACATCAGCGCCGCCCAG |
| | | ATCACCATGTGCCCACACGTGATCGTGGACGTGCGGCA |
| | | GCTGGAGCCCGTGCTGCTGCCACTGCCCGACATCCGGA |
| | | ACCGGTTCTTCCACTACAACCAGGAGAACACCCCACGG |
| | | ATGCGGCTGGTGGCCATGCTGTACACCCCACTGCGGGC |
| | | CAACAGCGGCGAGGACGTGTTCACCGTGAGCTGCCGG |
| | | GTGCTGACCAGGCCAGCCCCAGACTTCGAGTTCACCTT |
| | | CCTGGTGCCACCCACCGTGGAGAGCAAGACCAAGCCC |
| | | TTCACCCTGCCCATCCTGACCCTGGGCGAGCTGAGCAA |
| | | CAGCCGGTTCCCAGCCCCAATCGACATGCTGTACACCG |
| | | ACCCAAACGAGGCCATCGTGGTGCAGCCACAGAACGG |
| | | CCGGTGCACCCTGGACGGCACCCTGCAGGGCACCACCC |
| | | AGCTGGTGCCCACCCAGATCTGCAGCTTCCGGGGCACC |
| | | CTGATCAGCCAGACCAGCCGGAGCGCAGACAGCACCG |
| | | ACAGCGCCCCACGGGTGCGGAACCACCCACTGCACGT |
| | | GCAGCTGAAGAACCTGGACGGCACCCCATACGACCCA |
| | | ACCGACGAGGTGCCCGCAGTGCTGGGCGCCATCGACTT |
| | | CAAGGGCACCGTGTTCGGCATCGCCAGCCAGCGGAAC |
| | | ACCACCGGCAGCAGCATCGGAGCCACCCGGGCACACG |
| | | AGGTGCACATCGACACCACCAACCCACGGTACACCCC |
| | | AAAGCTGGGCAGCGTGCTGATGTACAGCGAGAGCAAC |
| | | GACTTCGACGACGGCCAGCCCACCCGGTTCACCCCAAT |
| | | CGGCATGGGCGCCGACGACTGGCGGCAGTGGGAGCTG |
| | | CCCGAGTACAGCGGCCACCTGACCCTGAACATGAACCT |
| | | GGCCCCAGCCGTGGCACCAGCCTTCCCAGGCGAGCGG |
| | | ATCCTGTTCTTCCGGAGCGTGGTGCCCAGCGCAGGAGG |
| | | CTACGGCAGCGGCCACATCGACTGCCTGATCCCACAGG |
| | | AGTGGGTGCAGCACTTCTACCAGGAGGCCGCACCAAG |
| | | CCAGAGCGCCGTGGCCCTGATCCGGTACGTGAACCCAG |
| | | ACACCGGCCGGAACATCTTCGAGGCCAAGCTGCACCG |
| | | GGAGGGCTTCATCACCGTGGCCAACAGCGGCAACAAC |
| | | CCAATCGTGGTGCCACCCAACGGCTACTTCCGGTTCGA |
| | | GGCCTGGGTGAACCAGTTCTACACCCTGACCCCAATGG |
| | | GCACCGGACAGGGACGGAGGCGGGTGCAG |
| 12 | GII.2 | ATGAAGATGGCCAGCAACGACGCCGCACCAAGCACCG |
| | Organism: | ACGGAGCCGCAGGACTGGTGCCCGAGAGCAACAACGA |
| | Synthetic | GGTGATGGCCCTGGAGCCCGTGGCAGGAGCCGCACTG |
| | construct | GCAGCACCAGTGACCGGCCAGACCAACATCATCGACC |
| | Mol. Type: | CATGGATCCGGGCCAACTTCGTGCAGGCCCCAAACGGC |
| | Other DNA | GAGTTCACCGTGAGCCCACGGAACGCCCCAGGCGAGG |
| | | TGCTGCTGAACCTGGAGCTGGGCCCAGAGCTGAACCCA |
| | | TACCTGGCCCACCTGGCCCGGATGTACAACGGCTACGC |
| | | CGGCGGCATGGAGGTGCAGGTGATGCTGGCCGGCAAC |
| | | GCCTTCACCGCCGGCAAGCTGGTGTTCGCCGCCGTGCC |
| | | ACCACACTTCCCAGTGGAGAACCTGAGCCCACAGCAG |
| | | ATCACCATGTTCCCACACGTGATCATCGACGTGCGGAC |
| | | CCTGGAGCCCGTGCTGCTGCCACTGCCCGACGTGCGGA |
| | | ACAACTTCTTCCACTACAACCAGAAGGACGACCCAAA |
| | | GATGCGGATCGTGGCCATGCTGTACACCCCACTGCGGA |
| | | GCAACGGCAGCGGCGACGACGTGTTCACCGTGAGCTG |
| | | CCGGGTGCTGACCAGGCCCAGCCCAGACTTCGACTTCA |
| | | CCTACCTGGTGCCACCCACCGTGGAGAGCAAGACCAA |
| | | GCCCTTCACCCTGCCCATCCTGACCCTGGGCGAGCTGA |
| | | GCAACAGCCGGTTCCCAGTGAGCATCGACCAGATGTAC |
| | | ACCAGCCCAAACGAGATCATCAGCGTGCAGTGCCAGA |
| | | ACGGCCGGTGCACCCTGGACGGCGAGCTGCAGGGCAC |
| | | CACCCAGCTGCAGGTGAGCGGCATCTGCGCCTTCAAGG |
| | | GCGAGGTGACCGCCCACCTGCACGACAACGACCACCT |
| | | GTACAACGTGACCATCACCAACCTGAACGGCAGCCCAT |
| | | TCGACCCAAGCGAGGACATCCCAGCCCCACTGGGCGT |
| | | GCCCGACTTCCAGGGCCGGGTGTTCGGCATCATCAGCC |
| | | AGCGGGACAAGCACAACAGCCCAGGCCACAACGAGCC |
| | | CGCCAACCGGGGACACGACGCCGTGGTGCCCACCTAC |
| | | ACCGCCCAGTACACCCCAAAGCTGGGCCAGATCCAGA |
| | | TCGGCACCTGGCAGACCGACGACCTGACCGTGAACCA |
| | | GCCCGTGAAGTTCACCCCAGTGGGCCTGAACGACACCG |
| | | AGCACTTCAACCAGTGGGTGGTGCCACGGTACGCCGGC |
| | | GCCCTGAACCTGAACACCAACCTGGCCCCAAGCGTGGC |
| | | CCCAGTGTTCCCAGGCGAGCGGCTGCTGTTCTTCCGGA |
| | | GCTACATCCCACTGAAGGGCGGCTACGGCAACCCAGC |
| | | CATCGACTGCCTGCTGCCACAGGAGTGGGTGCAGCACT |
| | | TCTACCAGGAGGCCGCCCCAAGCATGAGCGAGGTGGC |
| | | CCTGGTGCGGTACATCAACCCAGACACCGGCCGGGCCC |
| | | TGTTCGAGGCCAAGCTGCACCGGGCCGGCTTCATGACC |
| | | GTGAGCAGCAACACCAGCGCCCCAGTGGTGGTGCCCG |
| | | CCAACGGCTACTTCCGGTTCGACAGCTGGGTGAACCAG |
| | | TTCTACAGCCTGGCCCCAATGGGCACCGGCAACGGACG |
| | | GAGGCGGGTGCAG |
| 13 | GII.3 | ATGAAGATGGCCAGCAACGACGCCACCCCAAGCAACG |
| | Organism: | ACGGAGCCGCAGGCCTGGTGCCCGAGATCAACAACGA |
| | Synthetic | GGCCATGGCCCTGGAGCCCGTGGCAGGAGCAGCCATC |
| | construct | GCCGCACCACTGACCGGCCAGCAGAACATCATCGACC |
| | Mol. Type: | CATGGATCATGAACAACTTCGTGCAGGCCCCAGGCGGC |
| | Other DNA | GAGTTCACCGTGAGCCCACGGAACAGCCCAGGCGAGG |
| | | TGCTGCTGAACCTGGAGCTGGGCCCAGAGATCAACCCA |
| | | TACCTGGCCCACCTGGCCCGGATGTACAACGGCTACGC |
| | | CGGCGGCTTCGAGGTGCAGGTGGTGCTGGCCGGCAAC |
| | | GCCTTCACCGCCGGCAAGATCATCTTCGCCGCCATCCC |
| | | ACCCAACTTCCCAATCGACAACCTGAGCGCCGCCCAGA |
| | | TCACCATGTGCCCACACGTGATCGTGGACGTGCGGCAG |
| | | CTGGAGCCCGTGAACCTGCCCATGCCCGACGTGCGGAA |
| | | CAACTTCTTCCACTACAACCAGGGAAGCGACAGCCGGC |
| | | TGCGGCTGGTGGCCATGCTGTACACCCCACTGCGGGCC |
| | | AACAACAGCGGCGACGACGTGTTCACCGTGAGCTGCC |
| | | GGGTGCTGACCAGGCCCAGCCCAGAGTTCAGCTTCAAC |
| | | TTCCTGGTGCCACCCACCGTGGAGAGCAAGACCAAGCC |
| | | CTTCACCCTGCCCATCCTGACCATCAGCGAGATGAGCA |
| | | ACAGCCGGTTCCCAGTGCCCATCGACAGCCTGCACACC |
| | | AGCCCAACCGAGAACATCGTGGTGCAGTGCCAGAACG |
| | | GCCGGGTGACCCTGGACGGCGAGCTGATGGGCACCAC |
| | | CCAGCTGCTGCCCAGCCAGATCTGCGCCTTCCGGGGAG |
| | | TGCTGACCCGGAGCACCAGCCGGGCAAGCGACCAGGC |
| | | AGACACCGCCACCCCACGGCTGTTCAACTACTACTGGC |
| | | ACATCCAGCTGGACAACCTGAACGGCACCCCATACGA |
| | | CCCAGCCGAGGACATCCCAGGCCCACTGGGCACCCCA |
| | | GACTTCCGGGGCAAGGTGTTCGGCGTGGCCAGCCAGC |
| | | GGAACCCAGACAGCACCACCCGGGCCCACGAGGCCAA |
| | | GGTGGACACCACCGCCGGCCGGTTCACCCCAAAGCTG |
| | | GGCAGCCTGGAGATCAGCACCGAGAGCGACGACTTCG |
| | | ACCAGAACCAGCCCACCCGGTTCACCCCAGTGGGCATC |
| | | GGCGTGGACAACGAGGCCGACTTCCAGCAGTGGAGCC |
| | | TGCCCGACTACAGCGGCCAGTTCACCCACAACATGAAC |
| | | CTGGCCCCAGCCGTGGCCCCAAACTTCCCAGGCGAGCA |
| | | GCTGCTGTTCTTCCGGAGCCAGCTGCCCAGCAGCGGAG |
| | | GACGGAGCAACGGCATCCTGGACTGCCTGGTGCCACA |
| | | GGAGTGGGTGCAGCACTTCTACCAGGAGAGCGCACCA |
| | | GCCCAGACCCAGGTGGCCCTGGTGCGGTACGTGAACCC |
| | | AGACACCGGCCGGGTGCTGTTCGAGGCCAAGCTGCAC |
| | | AAGCTGGGCTTCATGACCATCGCCAAGAACGGCGACA |
| | | GCCCAATCACCGTGCCACCCAACGGCTACTTCCGGTTC |
| | | GAGAGCTGGGTGAACCCATTCTACACCCTGGCCCCAAT |
| | | GGGCACCGGCAACGGACGGAGGCGGGTGCAG |
| 14 | GII.4 | MKMASSDANPSDGSAANLVPEVNNEVMALEPVVGAAIA |
| | Organism: | APVAGQQNVIDPWIRNNFVQAPGGEFTVSPRNAPGEILWS |
| | Synthetic | APLGPDLNPYLSHLARMYNGYAGGFEVQVILAGNAFTAG |
| | construct | KIIFAAVPPNFPTEGLSPSQVTMFPHIIVDVRQLEPVLIPLP |
| | Mol. Type: | DVRNNFYHYNQSNDSTIKLIAMLYTPLRANNAGDDVFTV |
| | Other Protein | SCRVLTRPSPDFDFIFLVPPTVESRTKPFSVPVLTVEEMTN |
| | | SRFPIPLEKLFTGPSSAFVVQPQNGRCTTDGVLLGTTQLSP |
| | | VNICTFRGDVTHITGSRNYTMNLASQNWNNYDPTEEIPAP |
| | | LGTPDFVGKIQGMLTQTTRTDGSTRGHKATVYTGSADFA |
| | | PKLGRVQFETDTDHDFEANQNTKFTPVGVIQDGSTTHRN |
| | | EPQQWVLPSYSGRNTHNVHLAPAVAPTFPGEQLLFFRST |
| | | MPGCSGYPNMDLDCLLPQEWVQYFYQEAAPAQSDVALL |
| | | RFVNPDTGRVLFECKLHKSGYVTVAHTGQHDLVIPPNGY |
| | | FRFDSWVNQFYTLAPMGNGTGRRRVV |
| 15 | GI.1 | MMMASKDATSSVDGASGAGQLVPEVNASDPLAMDPVA |
| | Organism: | GSSTAVATAGQVNPIDPWIINNFVQAPQGEFTISPNNTPGD |
| | Synthetic | VLFDLSLGPHLNPFLLHLSQMYNGWVGNMRVRIMLAGN |
| | construct | AFTAGKIIVSCIPPGFGSHNLTIAQATLFPHVIADVRTLDPI |
| | Mol. Type: | EVPLEDVRNVLFHNNDRNQQTMRLVCMLYTPLRTGGGT |
| | Other Protein | GDSFVVAGRVMTCPSPDFNFLFLVPPTVEQKTRPFTLPNL |
| | | PLSSLSNSRAPLPISSMGISPDNVQSVQFQNGRCTLDGRLV |
| | | GTTPVSLSHVAKIRGTSNGTVINLTELDGTPFHPFEGPAPI |
| | | GFPDLGGCDWHINMTQFGHSSQTQYDVDTTPDTFVPHLG |
| | | SIQANGIGSGNYVGVLSWISPPSHPSGSQVDLWKIPNYGSS |
| | | ITEATHLAPSVYPPGFGEVLVFFMSKMPGPGAYNLPCLLP |
| | | QEYISHLASEQAPTVGEAALLHYVDPDTGRNLGEFKAYP |
| | | DGFLTCVPNGASSGPQQLPINGVFVFVSWVSRFYQLKPV |
| | | GTASSARGRLGLRR |
| 16 | GII.6 | MKMASNDAAPSNDGAANLVPEATNEVMALEPVVGASIA |
| | Organism: | APVVGQQNIIDPWIRENFVQAPQGEFTVSPRNSPGEMLLN |
| | Synthetic | LELGPELNPYLGHLSRMYNGYAGGMQVQVVLAGNAFTA |
| | construct | GKIIFAAVPPHFPVENISAAQITMCPHVIVDVRQLEPVLLP |
| | Mol. Type: | LPDIRNRFFHYNQENTPRMRLVAMLYTPLRANSGEDVFT |
| | Other Protein | VSCRVLTRPAPDFEFTFLVPPTVESKTKPFTLPILTLGELSN |
| | | SRFPAPIDMLYTDPNEAIVVQPQNGRCTLDGTLQGTTQLV |
| | | PTQICSFRGTLISQTSRSADSTDSAPRVRNHPLHVQLKNLD |
| | | GTPYDPTDEVPAVLGAIDFKGTVFGIASQRNTTGSSIGATR |
| | | AHEVHIDTTNPRYTPKLGSVLMYSESNDFDDGQPTRFTPI |
| | | GMGADDWRQWELPEYSGHLTLNMNLAPAVAPAFPGERI |
| | | LFFRSVVPSAGGYGSGHIDCLIPQEWVQHFYQEAAPSQSA |
| | | VALIRYVNPDTGRNIFEAKLHREGFITVANSGNNPIVVPPN |
| | | GYFRFEAWVNQFYTLTPMGTGQGRRRVQ |
| 17 | GII.2 | MKMASNDAAPSTDGAAGLVPESNNEVMALEPVAGAAL |
| | Organism: | AAPVTGQTNIIDPWIRANFVQAPNGEFTVSPRNAPGEVLL |
| | Synthetic | NLELGPELNPYLAHLARMYNGYAGGMEVQVMLAGNAF |
| | construct | TAGKLVFAAVPPHFPVENLSPQQITMFPHVIIDVRTLEPVL |
| | Mol. Type: | LPLPDVRNNFFHYNQKDDPKMRIVAMLYTPLRSNGSGDD |
| | Other Protein | VFTVSCRVLTRPSPDFDFTYLVPPTVESKTKPFTLPILTLGE |
| | | LSNSRFPVSIDQMYTSPNEIISVQCQNGRCTLDGELQGTTQ |
| | | LQVSGICAFKGEVTAHLHDNDHLYNVTITNLNGSPFDPSE |
| | | DIPAPLGVPDFQGRVFGIISQRDKHNSPGHNEPANRGHDA |
| | | VVPTYTAQYTPKLGQIQIGTWQTDDLTVNQPVKFTPVGL |
| | | NDTEHFNQWVVPRYAGALNLNTNLAPSVAPVFPGERLLF |
| | | FRSYIPLKGGYGNPAIDCLLPQEWVQHFYQEAAPSMSEV |
| | | ALVRYINPDTGRALFEAKLHRAGFMTVSSNTSAPVVVPA |
| | | NGYFRFDSWVNQFYSLAPMGTGNGRRRVQ |
| 18 | GII.3 | MKMASNDATPSNDGAAGLVPEINNEAMALEPVAGAAIA |
| | Organism: | APLTGQQNIIDPWIMNNFVQAPGGEFTVSPRNSPGEVLLN |
| | Synthetic | LELGPEINPYLAHLARMYNGYAGGFEVQVVLAGNAFTA |
| | construct | GKIIFAAIPPNFPIDNLSAAQITMCPHVIVDVRQLEPVNLP |
| | Mol. Type: | MPDVRNNFFHYNQGSDSRLRLVAMLYTPLRANNSGDDV |
| | Other Protein | FTVSCRVLTRPSPEFSFNFLVPPTVESKTKPFTLPILTISEMS |
| | | NSRFPVPIDSLHTSPTENIVVQCQNGRVTLDGELMGTTQL |
| | | LPSQICAFRGVLTRSTSRASDQADTATPRLFNYYWHIQLD |
| | | NLNGTPYDPAEDIPGPLGTPDFRGKVFGVASQRNPDSTTR |
| | | AHEAKVDTTAGRFTPKLGSLEISTESDDFDQNQPTRFTPV |
| | | GIGVDNEADFQQWSLPDYSGQFTHNMNLAPAVAPNFPGE |
| | | QLLFFRSQLPSSGGRSNGILDCLVPQEWVQHFYQESAPAQ |
| | | TQVALVRYVNPDTGRVLFEAKLHKLGFMTIAKNGDSPIT |
| | | VPPNGYFRFESWVNPFYTLAPMGTGNGRRRVQ |

### Example 2: Preparation of Purified mRNA for Five Norovirus VP1 Encoded Sequences

Five purified DNA plasmids were generated by GenScript Probio Biotech and were constructed in a pUC57 backbone with a T7 promoter sequence, an encoded 5′ UTR sequence (SEQ ID NO: 6), GII.4, GI.1, GII.6, GII.2, or GII.3 norovirus VP1 DNA sequences (SEQ ID NO: 9-13), encoding for GII.4, GI.1, GII.6, GII.2, or GII.3 norovirus VP1 mRNA CDSs (SEQ ID NO: 1-5), and an encoded 3′ UTR sequence (SEQ ID NO: 7) with an encoded poly(A) tail. DNA plasmids were linearized using BbsI-HF restriction enzyme (New England Biolabs) for plasmids encoding for GII.4, GII.6, GII.2, or GII.3 (SEQ ID NO: 9, 11-13) and BspQI restriction enzyme (New England Biolabs) for the plasmid encoding for GI.I (SEQ ID NO: 10). Approximately 1 mg of plasmid was added to a linearization reaction mixture at a concentration of about 0.2-0.4 mg/mL with 1-2 U/mL of the appropriate restriction enzyme in a digest buffer (New England Biolabs) and incubated for about 1 hr at 37° C. Linearization reactions were purified by isopropanol and ethanol precipitation steps to generate a purified linear DNA template with a concentration of about 0.8-1.2 mg/mL. Five IVT reactions were performed at a 10 mL scale in a 60 mL High Density Polyethylene (HDPE) bottle (Nalgene) mixed in a shaking incubator (Benchmark Scientific) set to a temperature of about 37° C. The purified linear DNA template was added to the IVT reaction mixtures at a concentration of about 40-60 μg/mL. ATP, GTP, and CTP (New England Biolabs) were added to the IVT reaction mixture at a concentration range of about 8-14 mM, m¹Ψ (BOC Sciences) was fully substituted for UTP and added at about 4-6 mM, and CleanCap AG (3′OMe) (TriLink Biotechnologies) was added at about 3-5 mM. T7 RNA polymerase (New England Biolabs) was added to the IVT reaction mixtures at a concentration of about 10,000-12,000 U/mL, yeast inorganic pyrophosphatase (New England Biolabs) was added at about 2 U/mL, and murine RNase inhibitor (New England Biolabs) was added at 1 U/mL. The IVT reaction mixtures were prepared in a transcription buffer matrix similar to 1X T7 Transcription Buffer (New England Biolabs) containing tris hydrochloride, magnesium, spermidinc, DTT, and other excipients at a final solution pH of about 6-9. The IVT reaction mixtures were incubated with mixing for 3-4 hr and the reaction was terminated by addition of DNase I (New England Biolabs) at a concentration range of about 350 U/mL along with calcium chloride (Sigma Aldrich) at a concentration of 3.5 mM, and incubated at 37° C. for 1 hr.

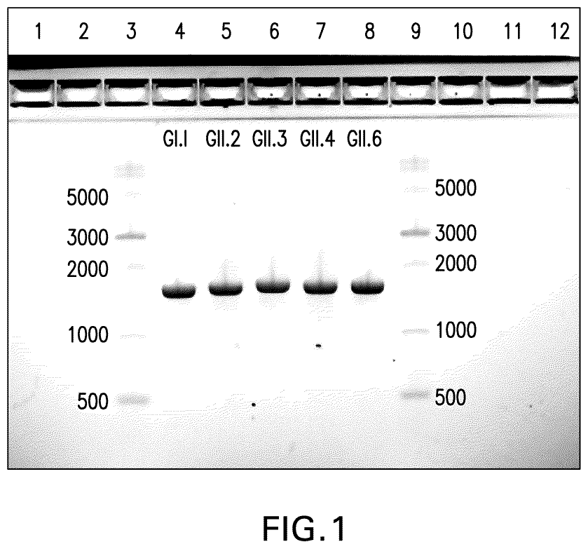

The five IVT reaction mixtures were quenched with about 50 mM EDTA (Thermo Fisher Scientific) and diluted to approximately 300 mL in an oligo dT binding matrix consisting of 400 mM sodium chloride (Thermo Fisher Scientific), 10 mM tris hydrochloride (Thermo Fisher Scientific), 2 mM EDTA, pH 7.2. The five diluted IVT reaction mixtures were purified using 40 mL CIMmultus oligo dT monolithic columns (Sartorius AG) at a residence time of about 0.5 min. After hybridization of loaded mRNA with a poly(A) tail to the oligo dT ligand, the column was washed with 5 CVs of a buffer consisting of 50 mM sodium chloride, 10 mM tris hydrochloride, 2 mM EDTA, pH 7.2, and the bound mRNA was cluted with 5 CVs of 10 mM tris hydrochloride, pH 7.2. The five oligo dT elution fractions were loaded to 235 cm² 50 kDa hollow fiber PES membranes (Repligen) and operated at a TMP of about 4 psi and a crossflow shear rate of 4000 s⁻¹ to concentrate about 8-fold and buffer exchange 6 DVs into 1 mM sodium citrate (Thermo Fisher Scientific), pH 6.4. The final TFF retentates were pumped at a flux of 300 L/m²-hr through an 18 cm² 0.2 μm PVDF (Millipore) bioburden reduction filter. The bioburden reduction filter product mRNA concentrations were calculated from a sample measurement of absorbance at 260 nm using a spectrophotometer and the product was diluted with 1 mM sodium citrate, pH 6.4 to about 1 mg/mL, filled into conical tube containers, and stored <−60° C. The final mRNA yields purified from 10 mL IVT reactions were 85 mg, 78 mg, 89 mg, 75 mg, and 78 mg for GII.4, GI.I, GII.6, GII.2, and GII.3 mRNAs, respectively. The purified mRNAs were analyzed by agarose gel electrophoresis using a 2% agarose E-Gel and Power Snap electrophoresis system (Thermo Fisher Scientific) and the expected size and purity were observed. FIG. 1 shows an image of a 2% agarose E-gel of purified mRNA for five norovirus VP1 encoded sequences with 50 ng mRNA loading per lane. Lanes 1-2 and 10-12 are blank, lanes 3 and 9 contain an ssRNA ladder (New England Biolabs), lane 4 contains GI.I, lane 5 contains GII.2, lane 6 contains GII.3, lane 7 contains GII.4, and lane 8 contains GII.6.

### Example 3: LNP Encapsulation of mRNAs

mRNA/LNP Preparation

LNPs encapsulating one or up to five different norovirus mRNA constructs (e.g., GI.1, GII.2, GII.3, GII.4, and/or GII.6) can be formed via rapid precipitation using a microfluidics mixer, a multi-inlet vortex mixer or T-mixer to micro-mix two fluid streams. One fluid stream contains lipids dissolved in ethanol and the other fluid stream is an aqueous solution containing mRNA. The lipid solution prepared in ethanol contains cationic lipid, cholesterol, PEG-DMG, and phospholipid (DSPC) at specified molar ratios and is heated to a temperature in the range of 25-45° C. The mRNA solution, prepared to achieve a target mRNA wt %, consists of a sodium citrate buffered salt solution with pH in the range of 4-6 and is kept at a temperature in the range of 16-25° C. The lipid molar ratios and mRNA wt % are selected to achieve a nitrogen-to-phosphate (N/P) ratio in the range of 2-9, targeting 5-7. The lipid and mRNA solutions are micro-mixed to instantly form LNPs encapsulating mRNA (mRNA/LNPs).

For microfluidic mixed mRNA/LNPs, the mRNA and lipid containing solution streams are combined at 8-15 mL/min in a 1:1 to 5:1 ratio to produce 16-50% vol:vol alcohol in the mixed solution and to achieve an N/P ratio in the range of 2-9, targeting 5-7. The resulting mRNA/LNP suspension then undergoes buffer exchange and alcohol removal via dialysis. The mRNA/LNP suspension is dialyzed 2-4× against the final buffered solution at a pH range of 7-8. The final buffered solution may contain a cryoprotectant (e.g., containing sucrose, trehalose, or a combination). The dialysis step can use a membrane nominal molecular weight cutoff range from 30-500 kDa. Before terminal sterile filtration, the mRNA/LNP suspension may be combined with additional norovirus mRNA/LNP suspensions encoding VP1 proteins from different norovirus genotypes (e.g., GI.1, GII.2, GII.3, GII.4, and/or GII.6) to form a co-formulated bivalent, trivalent, quadrivalent, or pentavalent product. Monovalent or co-formulated mRNA/LNP suspensions are then filtered through 0.2 μm polyethersulfone (PES) or polyvinylidene fluoride (PVDF) sterilizing filters into sterile glass vials and sealed. The resulting mRNA/LNP formulation can then be stored under refrigerated (2-8° C.) or frozen (−20±10° C. or ≤−60° C.) conditions. If monovalent mRNA/LNP formulations are prepared into sterile vials, these formulations can be equilibrated to a temperature in the range of 16-25° C. and combined with additional norovirus mRNA/LNP formulations encoding VP1 proteins from different norovirus genotypes (e.g., GI.1, GII.2, GII.3, GII.4, and/or GII.6) to form a co-formulated bivalent, trivalent, quadrivalent or pentavalent final vaccine prior to administration.

When micro-mixing using a confined volume T-mixer, the T-mixer has an internal diameter (ID) of 0.25 to 1.0 mm. The lipid and mRNA solutions are delivered to the inlet of the T-mixer using programmable pumps. The total flow rate at the inlet of the T-mixer ranges from 100-600 mL/min. The lipid and mRNA solutions are delivered to the T-mixer with a ratio in the range of 0.9:1 to 1:3 vol:vol, with a target range of 1:1.1, to 1:2 to produce a 16-53% vol:vol alcohol in the mixed solution and to achieve an N/P ratio in the range of 2-9, targeting 5-7. The resulting mRNA/LNP suspension is twice diluted into higher pH buffers in the pH range of 6-8 using a sequential, multi-stage in-line mixing process. The mRNA/LNP suspension is first diluted with a buffered solution at a higher pH (6-8) using a mixing ratio in the range of 1:1 to 1:3 vol:vol, targeting 1:2 vol:vol. This buffered solution is at a temperature in the range of 15-40° C., but targeting 30-40° C. The second dilution involves mixing the mRNA/LNP suspension with a buffered solution at a higher pH (pH 6-8) using a mixing ratio in the range of 1:1 to 1:3 vol:vol, targeting 1:2 vol:vol. The second dilution buffer solution is at a temperature in the range of 15-40° C., but targeting 16-25° C. The fully diluted mRNA/LNP suspension is held for 30 minutes to 4 hours at a temperature in the range of 15-40° C., but targeting 16-25° C., prior to undergoing anionic exchange filtration. Upon completion of the incubation step, the mRNA/LNP suspension is filtered through a 0.8 μm anionic exchange filter using tubing with IDs ranging from 1-10 mm and a flow rate in the range of 10-2000 mL/min. The mRNA/LNPs are concentrated and diafiltered using ultrafiltration in a tangential flow filtration (TFF) format to remove alcohol. The mRNA/LNP suspension is then buffer exchanged into the final buffered solution at a pH range of 7-8. The final buffered solution may be suitable for cryopreservation (e.g., containing sucrose, trehalose, or a combination). The TFF processes can use a hollow fiber or flat sheet membrane with a membrane nominal molecular weight cutoff range from 30-500 kDa, targeting 500 kDa, to retain the mRNA/LNPs in the retentate. The multi-step TFF process begins with a concentration step to achieve an mRNA concentration of 0.4-0.7 mg/mL followed by diafiltration against the final buffer (e.g., modified Dulbecco's Phosphate Buffered Saline (mDPBS) with pH of 7-8 or 10 mM Tris, 10% w/v Sucrose, pH 7-8) for 5-20 volumes to remove alcohol and perform buffer exchange. The resulting mRNA/LNP suspension is then concentrated an additional 1-5-fold using ultrafiltration. Once concentrated, the mRNA/LNP suspension undergoes bioburden reducing filtration using sequential 0.45 μm and 0.2 μm PES, cellulose acetate (CA), and/or PVDF filters. The bioburden reduced mRNA/LNP suspension is then stored under refrigerated (2-8° C.) or frozen (≤−60° C.) conditions until further use. Prior to terminal sterile filtration, the mRNA/LNP suspension is thawed in a water bath at a temperature in the range of 20-30° C. and may be combined with additional norovirus mRNA/LNP suspensions specific encoding VP1 proteins from different norovirus genotypes (e.g., GI.1, GII.2, GII.3, GII.4, and/or GII.6) to form a co-formulated bivalent, trivalent, quadrivalent, or pentavalent product. The resulting mRNA/LNP suspension can then be diluted to a target mRNA concentration prior to undergoing terminal sterile filtration using sequential 0.45 μm and 0.2 μm PES and/or PVDF filters and filled into vials. The terminally sterile filtered mRNA/LNP suspension can also be lyophilized. The final mRNA/LNP vials (liquid or lyophilized) are then stored under refrigerated (2-8° C.) or frozen (−20±10° C. or ≤−60° C.) conditions. Terminally sterilized monovalent norovirus mRNA/LNP formulations (GI.1, GII.2, GII.3, GII.4, or GII.6) in sterile vials can be thawed or reconstituted with diluent (e.g., saline) and combined with additional norovirus mRNA/LNP formulations encoding VP1 proteins from different norovirus genotypes (e.g., GI.1, GII.2, GII.3, GII.4, and/or GII.6) to form a co-formulated bivalent, trivalent, quadrivalent or pentavalent final vaccine for administration.

Preparation of mRNA/LNP Formulations

LNPs encapsulating one norovirus mRNA construct were formed via rapid precipitation using microfluidics or tee-mixers to micro-mix two fluid streams. One fluid stream contained lipids dissolved in ethanol and the other fluid stream was an aqueous solution containing mRNA. The lipid solution prepared in ethanol contained cationic lipid, cholesterol, PEG-DMG, and phospholipid (DSPC) at a specified molar ratio. The aqueous solution consisted of a 10 mM sodium citrate buffer, pH 4.5-5.5 and contained mRNA.

For microfluidic mixed LNPs, the mRNA and lipid containing solution streams were combined at 12 mL/min in a 3:1 ratio to produce a 25 vol:vol % alcohol in the mixed solution. The resulting LNP suspension had an N/P ratio in the range of 5-8. The mRNA/LNP suspension underwent buffer exchange and ethanol removal via dialysis. mRNA/LNP suspensions were dialyzed 3× against 20 mM Tris, 10% sucrose (w/v), pH 7.5 at volumes of 100-200× of the product using SpectraPor® Float-A-Lyzers® (Repligen) with a molecular weight cutoff of 100 kDa. The dialyzed mRNA/LNP suspension was then filtered through 0.2 μm polyethersulfone (PES; Pall) or polyvinylidene fluoride (PVDF; Millipore) sterile filters into sterile glass vials and scaled. The mRNA/LNP formulation was then stored under refrigerated (2-8° C.) or frozen (20° C. or ≤−60° C.) conditions. Prior to use, the mRNA/LNP vials were equilibrated to room temperature and either used as a monovalent vaccine or co-formulated with additional monovalent norovirus mRNA/LNP formulations encoding VP1 proteins from different norovirus genotypes (e.g., GI.1, GII.2, GII.3, GII.4, and/or GII.6) to form a co-formulated bivalent, trivalent, quadrivalent, or pentavalent vaccine.

For tee-mixer prepared mRNA/LNPs, the lipid containing solution stream was heated to a temperature in the range of 35-40° C. and then was mixed with the mRNA containing solution stream in a confined volume mixer (ID 0.5 mm) using programmable pumps. The total flow rate at the inlet of the t-mixer ranged from 100-150 ml/min and delivered the streams in a ratio of 0.90:1 to 1.3:1 to produce 43-53 vol-vol % alcohol in the mixed solution. The resulting mRNA/LNP suspension had an N/P ratio in the range of 5-8 and was diluted 2× into higher pH buffers (pH range 6-8) utilizing a sequential, multi-stage in-line mixing process. For the first dilution, the mRNA/LNP formulation was mixed with 20 mM sodium citrate, 300 mM sodium chloride, pH 6 at a mixing ratio of 1:1 vol:vol. The buffer solution was held at a temperature in the range of 35-40° C. The mRNA/LNP formulation was then further mixed with mDPBS, pH of 7.5 at a mixing ratio of 1:1 vol:vol. The mDPBS, pH 7.5 solution was at a temperature in the range of 16-25° C. The resulting mRNA/LNP suspension was held for 30 min in a temperature range of 16-25° C. prior to undergoing anionic exchange filtration. After filtration, the mRNA/LNP suspension was then concentrated and diafiltered via an ultrafiltration process to remove alcohol and perform a buffer exchange into the final buffer solution. TFF was used for the ultrafiltration process. A PES membrane using a hollow fiber format with a nominal molecular weight cutoff of 500 kDa was used for ultrafiltration. Ultrafiltration was first used to concentrate the mRNA/LNP suspension 6-8-fold by volume, targeting a mRNA concentration of 0.4-0.7 mg/mL. Alcohol was removed through subsequent diafiltration (10 diavolumes) using 20 mM Tris, 10% sucrose (w/v), pH 7.5. The mRNA/LNP suspension was then further concentrated 3-fold by volume targeting an mRNA concentration of 1.0-1.5 mg/mL. Once concentrated, the monovalent mRNA/LNP formulation underwent bioburden reducing filtration using sequential 0.45 μm and 0.2 μm PES, CA and/or PVDF filters. The bioburden reduced monovalent mRNA/LNP suspension is then stored under frozen (≤−60° C.) conditions until further use. Prior to terminal sterile filtration, the mRNA/LNP suspension was thawed using a water bath at a temperature range of 20-30° C. The thawed mRNA/LNP suspension was then sterile filtered using sequential 0.45 μm and 0.2 μm PES and/or PVDF filters. After filtration and under aseptic conditions, the mRNA/LNP suspension was diluted to a final target mRNA concentration utilizing sterile 20 mM Tris, 10% sucrose (w/v), pH 7.5, filled into sterile vials and sealed. The sealed mRNA/LNP containing vials were then stored under frozen conditions (≤−60° C.). Prior to administration, the mRNA/LNP vials were equilibrated to room temperature and either used as a monovalent vaccine or co-formulated with additional monovalent norovirus mRNA/LNP formulations encoding VP1 proteins from different norovirus genotypes (e.g., GI.1, GII.2, GII.3, GII.4, and/or GII.6) to form a co-formulated bivalent, trivalent, quadrivalent, or pentavalent vaccine.

### Example 4: In Vivo Immunization with LNP-Encapsulated mRNAs

### Mouse Studies

This study was designed to test the immunogenicity in mice of candidate norovirus vaccines. Animals tested were 6-8 week old BALB/c mice obtained from Charles River Laboratories. Test vaccines included the mRNA sequences encoding the VP1 proteins of 5 norovirus genotypes GI.1, GII.2, GII.3, GII.4, and GII.6 formulated in LNPs. Control animals were vaccinated with empty LNP.

At week 0 and week 4, animals were immunized intramuscularly with a total volume of 100 μL of each test vaccine, which was administered in a 50 μL immunization to each quadricep. Candidate vaccines evaluated in this study were described above and are outlined in Table 2 below. Sera were collected from all animals. Route of administration (ROA).

TABLE 2
Mouse dosing

| | No. of | | Dose level | | Volume | Dosing |
|---|---|---|---|---|---|---|
| Group | animals | Treatment | (μg) | ROA | (mL) | Schedule |
| 1 | 8 | LNP | 34 | IM | 0.1 | Weeks |
| | | | | | | 0, 4 |
| 2 | 8 | GI.1 | 1 | IM | 0.1 | Weeks |
| | | | | | | 0, 4 |
| 3 | 8 | GII.4 | 1 | IM | 0.1 | Weeks |
| | | | | | | 0, 4 |
| 4 | 8 | GII.2 | 1 | IM | 0.1 | Weeks |
| | | | | | | 0, 4 |
| 5 | 8 | GII.3 | 1 | IM | 0.1 | Weeks |
| | | | | | | 0, 4 |
| 6 | 8 | GII.6 | 1 | IM | 0.1 | Weeks |
| | | | | | | 0, 4 |
| 7 | 8 | GII.4 + | 1 + 1 + | IM | 0.1 | Weeks |
| | | GI.1 + | 1 + 1 + 1 | | | 0, 4 |
| | | GII.2 + | | | | |
| | | GII.3 + | | | | |
| | | GII.6 | | | | |
| | | (5-valent) | | | | |

To test the presence of antibodies capable of binding to VLP from norovirus, 384-well ELISA plates were each coated with 50 ng/well of one of the following VLP representing norovirus genotypes: GI.1, GII.2, GII.3, GII.4, and GII.6. While coating, a liquid handler made 10 point, 4-fold serial dilutions of the sera in blocking buffer. After coating, the ELISA plates were washed, incubated with blocking buffer, evacuated and then incubated with samples from serum dilution plate. After 2 hours incubation, ELISA plates were washed, and goat anti-mouse IgG (Fc)-HRP conjugate was added to each well. Plates were incubated 1 hour, washed, and incubated with chemiluminescent substrate for 15 minutes before having luminescence read (ultrasensitive, 0.1 seconds/well) on a plate reader. Interpolated endpoint titers were calculated as the highest dilution where relative luminescence (RLU) signal is above a 50,000 RLU cutoff. Samples where no dilution crossed the threshold were given a titer of “25,” for samples above the threshold at the highest dilution tested—1:13, 107, 200—the value 13,107,200 was used for the titer. Samples where a well value was exactly the same as 50,000 RLU were given the interpolated titer value at that exact dilution. Serum antibody titers were plotted as geometric mean titers with 95% confidence interval using GraphPad Prism software.

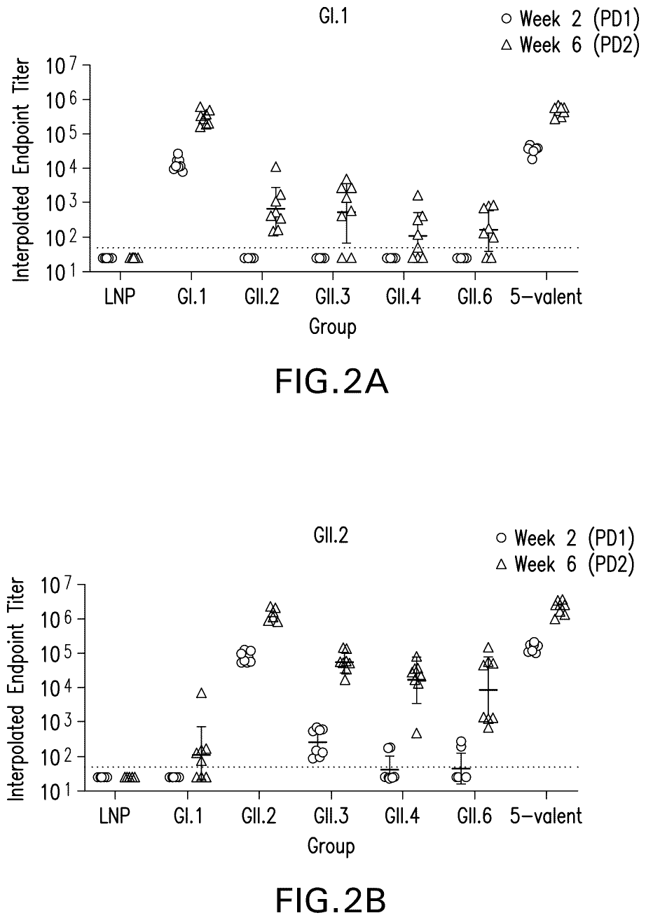

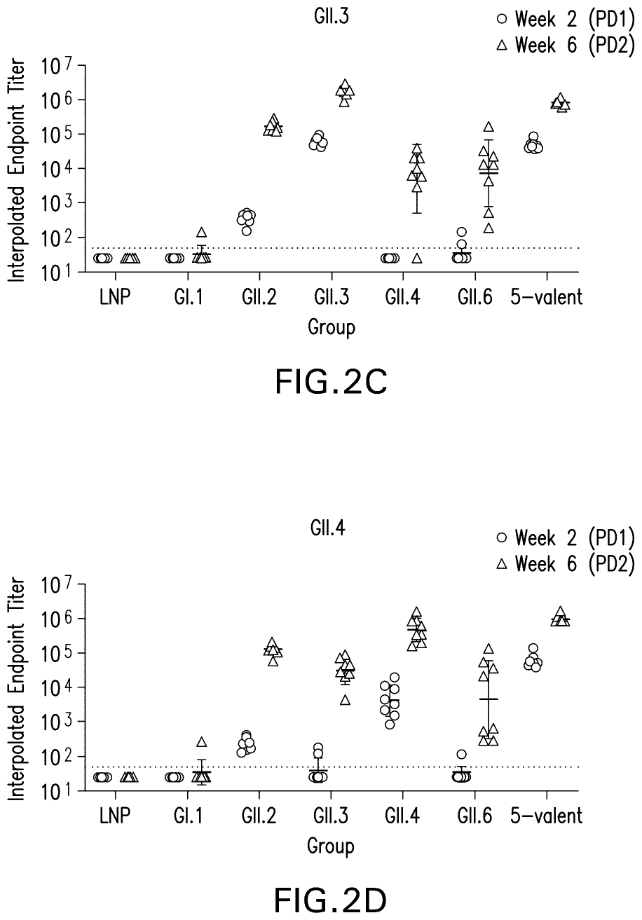

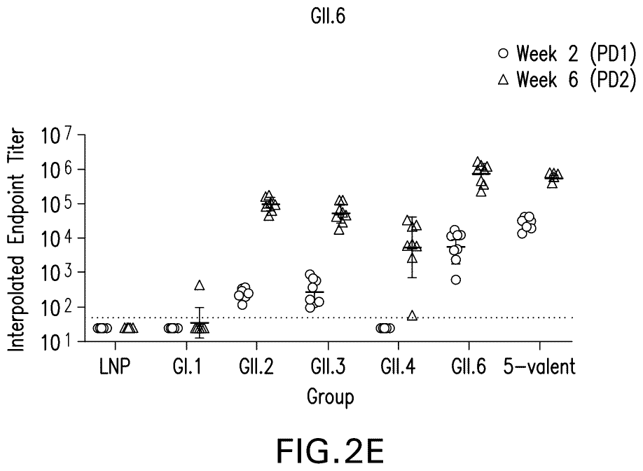

FIGS. 2A-2E depict the interpolated endpoint titers of sera samples from animals vaccinated with the test vaccines. The vaccine groups tested are shown on the x-axis and the binding to VLP from each of the different genotype of norovirus is plotted. The dotted line at 50 indicates the limit of detection. A value of 25 indicates a lack of binding, even at the lowest dilution tested. Compared to the monovalent vaccine candidate, administration of the pentavalent vaccine induced comparable serum IgG titers specific to the five norovirus genotypes GI.1, GII.2, GII.3, GII.4, and GII.6. The second dose of all the monovalent and the pentavalent vaccines boosted antibody titers by Week 6.

To probe the functional antibody response, the ability of serum at week 6 (post-dose 2) to block the binding of VLP to HBGA was assessed. 384-well HBGA plates were coated with 250 ng/well of porcine gastric mucin (PGM) or 1:1000 dilute human saliva, depending on the test genotype. While coating, a liquid handler made 10 point, 2-fold serial dilutions of the sera in blocking buffer. After dilution, 30 μL/well of GI.1, GII.2, GII.3, GII.4, or GII.6 VLP was added to each sample. After coating, the HBGA plates were washed, incubated with blocking buffer, evacuated and incubated with mixture from the serum dilution+VLP plate. After 2 hours incubation, the plates were washed, and rabbit serum hyperimmunized with corresponding VLP was added to each well. After 1 hour incubation, plates were washed, and goat anti-rabbit IgG (Fc fragment specific)-HRP was added. Plates were incubated 1 hour, washed, and incubated with chemiluminescent substrate for 15 minutes before having luminescence read (ultrasensitive, 0.1 seconds/well) on a plate reader. For the HBGA blocking assay, fifty-percent blocking titers (BT50), defined as the titer at which luminescence readings were 50% of the positive control, were determined for each sample. A value of 10 was assigned to samples with a BT50 less than 20. The BT50 values were plotted as the geometric mean with 95% confidence interval using Graph Pad Prism software.

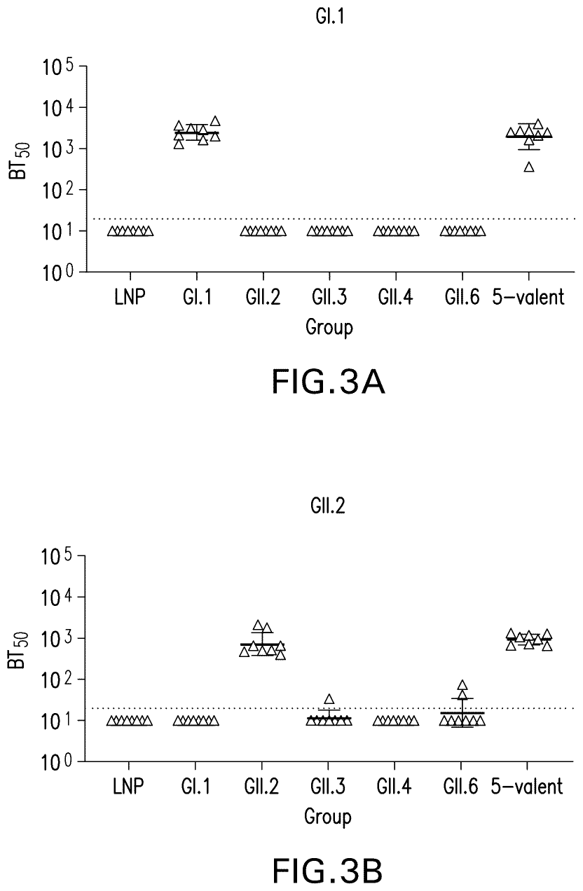

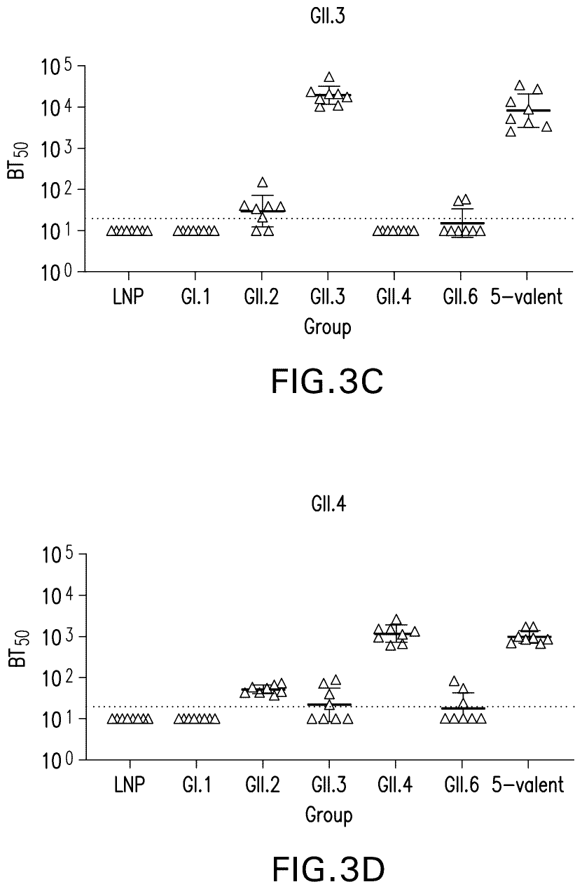

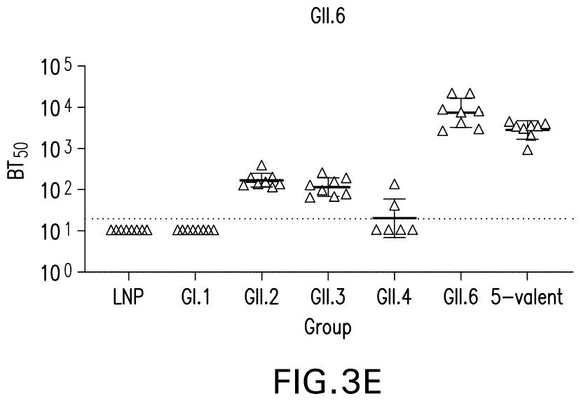

FIGS. 3A-3E depict the HBGA blocking titers of sera samples from animals vaccinated with the test vaccines. The vaccine groups tested are shown on the x-axis and BT50 against each of the five different genotype of norovirus is plotted. The dotted line at 20 indicates the limit of detection. A value of 10 indicates a lack of blocking, even at the lowest dilution tested. Compared to the monovalent vaccine candidate, administration of the pentavalent vaccine induced comparable HBGA blocking titers specific to the five norovirus genotypes GI.1, GII.2, GII.3, GII.4, and GII.6.

### Nonhuman Primate (NHP) Studies

This study was designed to test the immunogenicity in non-human primates of candidate norovirus vaccines. Animals tested were rhesus monkeys located at NIRC, New Iberia, Louisiana, US. Test vaccines included the mRNA sequences encoding the VP1 proteins of five norovirus genotypes GI.1, GII.2, GII.3, GII.4, and GII.6 formulated in LNPs. Control animals were vaccinated with empty LNP.

At week 0 and week 4, animals were immunized intramuscularly with a total volume of 500 μL of each test vaccine over posterior right thigh. Candidate vaccines evaluated in this study were described above and are outlined in Table 3 below. Sera were collected from all animals.

TABLE 3
NHP dosing

| | No. of | | Dose | ROA | Volume | Dosing |
|---|---|---|---|---|---|---|
| Group | animals | Treatment | level | ^{a} | (mL) | Schedule |
| 1 | 4 | LNP control | NA | IM | 0.5 | Weeks |
| | | | | | | 0, 4 |
| 2 | 4 | GII.4 | 40 μg | IM | 0.5 | Weeks |
| | | monovalent | | | | 0, 4 |
| 3 | 4 | GI.1, GII.2, | 50 μg | IM | 0.5 | Weeks |
| | | GII.3, GII.4, | | | | 0, 4 |
| | | and GII.6 | | | | |
| | | (Low dose, 10 | | | | |
| | | μg/genotype) | | | | |
| 4 | 4 | GI.1, GII.2, | 200 μg | IM | 0.5 | Weeks |
| | | GII.3, GII.4, | | | | 0, 4 |
| | | and GII.6 | | | | |
| | | (High dose, 40 | | | | |
| | | μg/genotype) | | | | |

To test the presence of antibodies capable of binding to VLP from norovirus, 384-well ELISA plates were each coated with 50 ng/well of one of GI.1, GII.2, GII.3, GII.4, or GII.6 VLP representing norovirus genotypes: GI.1, GII.2, GII.3, GII.4, and GII.6. While coating, a liquid handler made 10 point, 4-fold serial dilutions of the sera in blocking buffer. After coating, the ELISA plates were washed, incubated with blocking buffer, evacuated and then incubated with samples from serum dilution plate. After 2 hours incubation, ELISA plates were washed, and goat anti-human IgG (Fc fragment specific)-HRP conjugate was added to each well. Plates were incubated 1 hour, washed, and incubated with chemiluminescent substrate for 15 minutes before having luminescence read (ultrasensitive, 0.1 seconds/well) on a plate reader. Interpolated endpoint titers were calculated as the highest dilution where relative luminescence (RLU) signal is above a 50,000 RLU cutoff. Samples where no dilution crossed the threshold were given a titer of “25,” for samples above the threshold at the highest dilution tested—1:13, 107, 200—the value 13,107,200 was used for the titer. Samples where a well value was exactly the same as 50,000 RLU were given the interpolated titer value at that exact dilution. Serum antibody titers were plotted as geometric mean titers with 95% confidence interval using GraphPad Prism software.

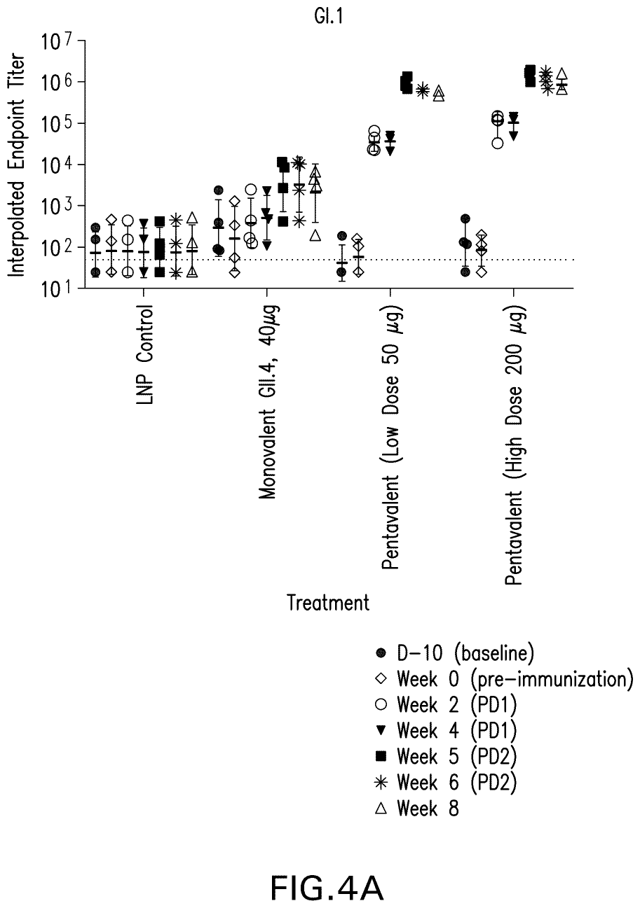

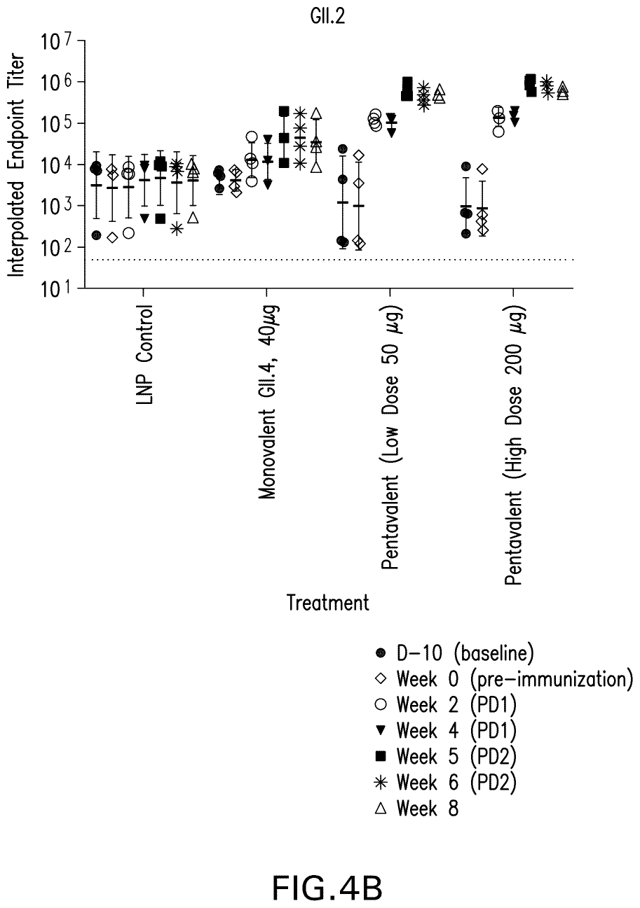

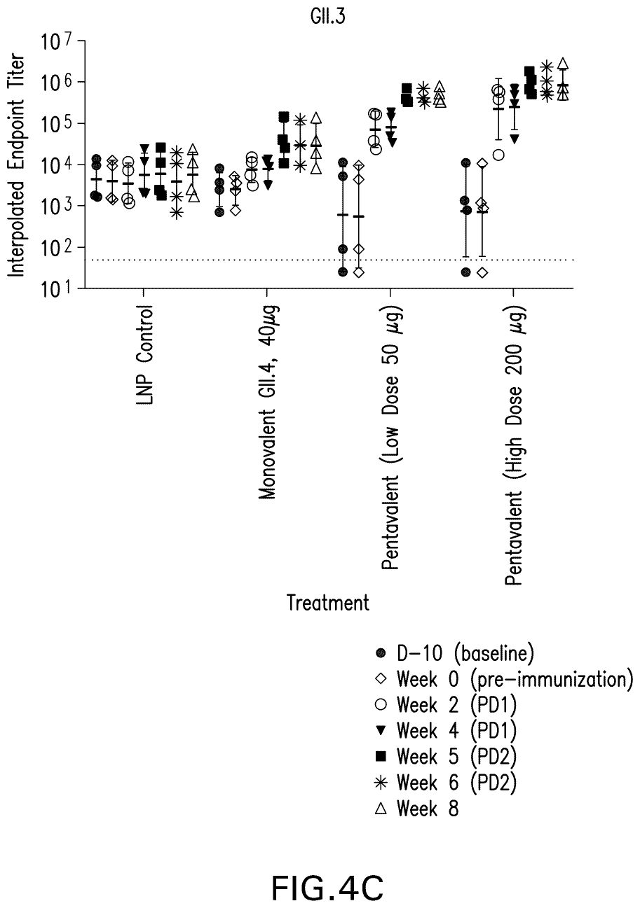

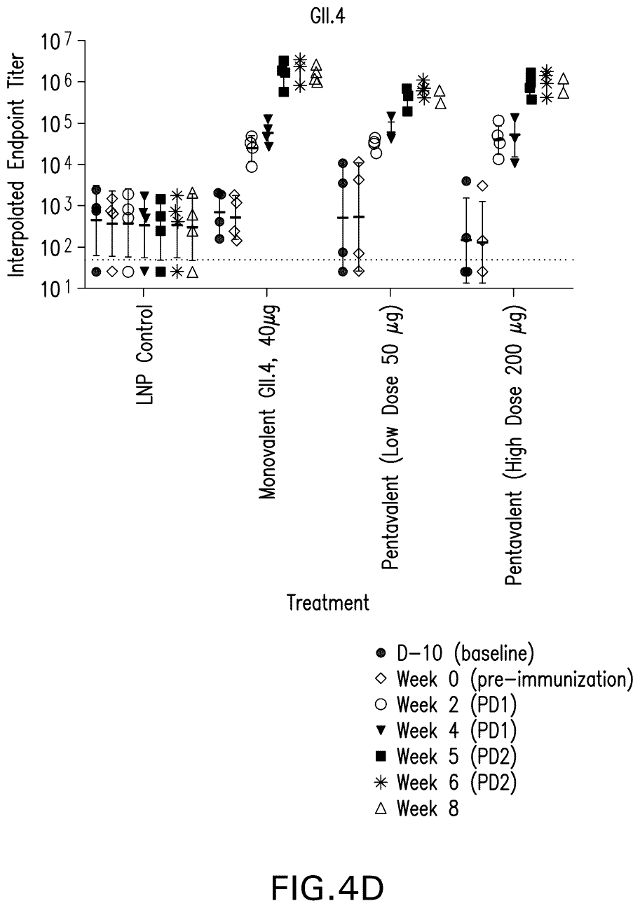

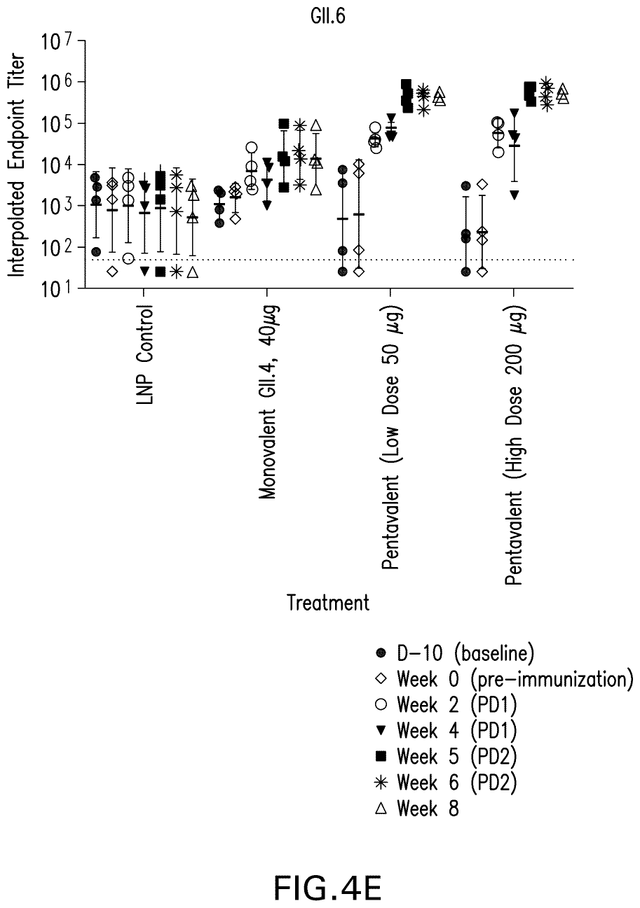

FIGS. 4A-4E depict interpolated endpoint titers of sera samples from animals vaccinated with the test vaccines. The vaccine groups tested are shown on the x-axis and the binding to VLP from each of the five different genotype of norovirus is plotted. The dotted line at 50 indicates the limit of detection. A value of 25 indicates a lack of binding, even at the lowest dilution tested. Inoculations at two dose levels of 50 μg or 200 μg of the pentavalent vaccine candidate elicited comparable and robust serum IgG binding antibody titers specific to the five norovirus genotypes GI.1, GII.2, GII.3, GII.4, and GII.6. Pentavalent vaccine candidates and monovalent GII.4 vaccine elicited comparable serum IgG ELISA titers against GII.4. The second dose of the pentavalent vaccine at both dose levels boosted antibody titers which remained stable out through Week 8.

To probe the functional antibody response, the ability of serum to block the binding of VLP to HBGA was assessed. 384-well HBGA plates were coated with 250 ng/well of porcine gastric mucin (PGM) or 1:1000 dilute human saliva, depending on the test genotype. While coating, a liquid handler made 10 point, 2-fold serial dilutions of the sera in blocking buffer. After dilution, 30 μL/well of one of GI.1, GII.2, GII.3, GII.4, or GII.6 VLP was added to each sample. After coating, the HBGA plates were washed, incubated with blocking buffer, evacuated and incubated with mixture from the serum dilution+VLP plate. After 2 hours incubation, the plates were washed, and rabbit serum hyperimmunized with corresponding VLP was added to each well. After 1 hour incubation, plates were washed, and goat anti-rabbit IgG (Fc fragment specific)-HRP was added. Plates were incubated 1 hour, washed, and incubated with chemiluminescent substrate for 15 minutes before having luminescence read (ultrasensitive, 0.1 seconds/well) on a plate reader. For the HBGA blocking assay, fifty-percent blocking titers (BT50), defined as the titer at which luminescence readings were 50% of the positive control, were determined for each sample. A value of 10 was assigned to samples with a BT50 less than 20. The BT50 values were plotted as the geometric mean with 95% confidence interval using Graph Pad Prism software.

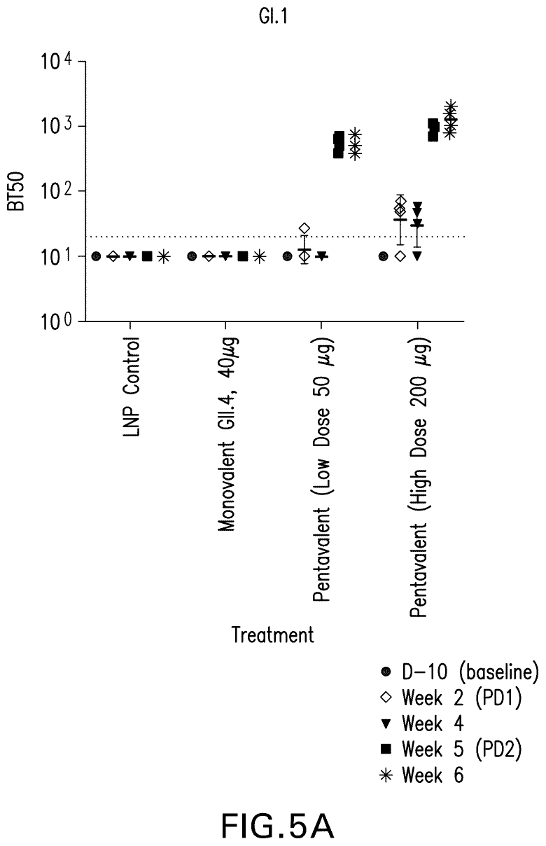

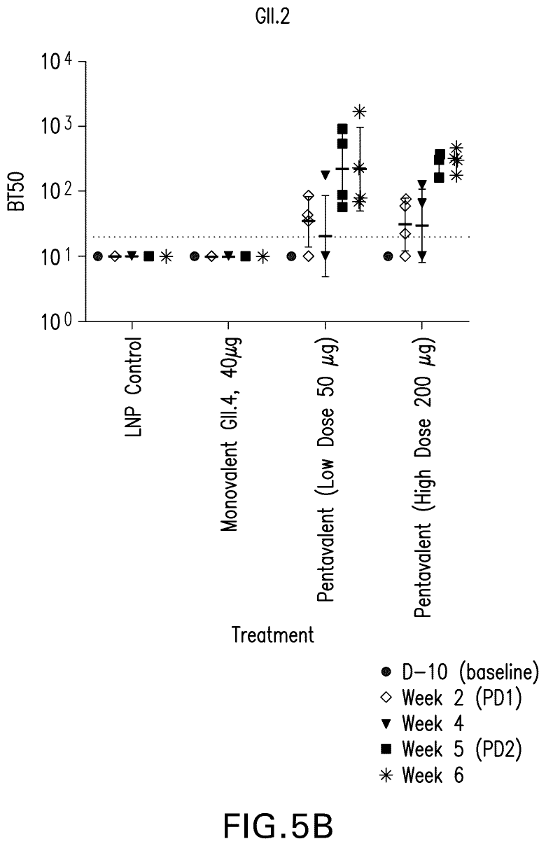

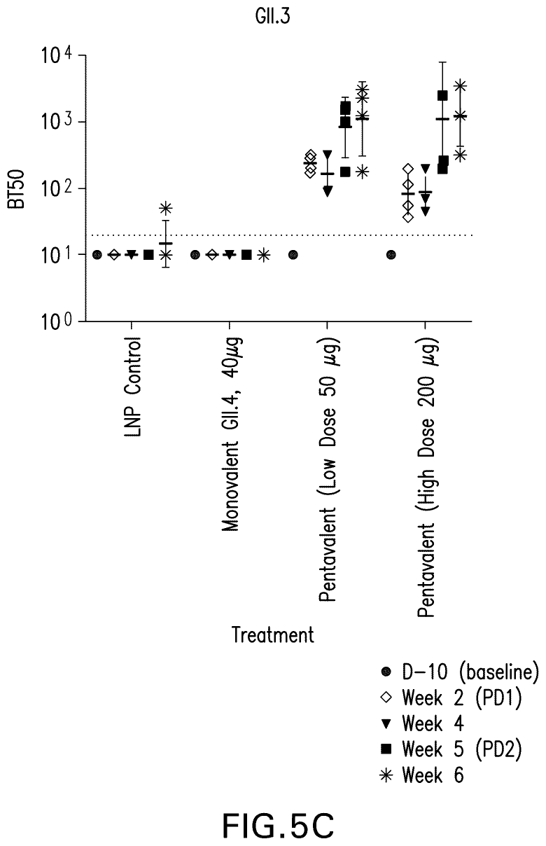

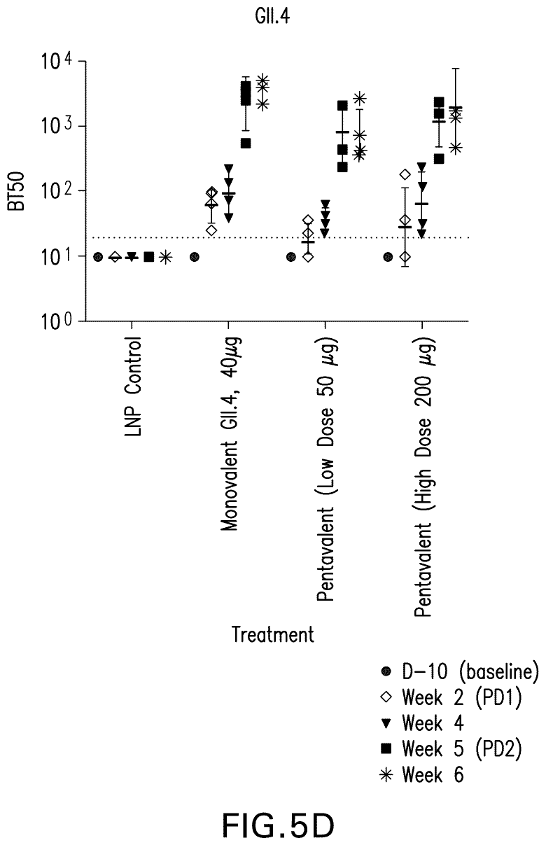

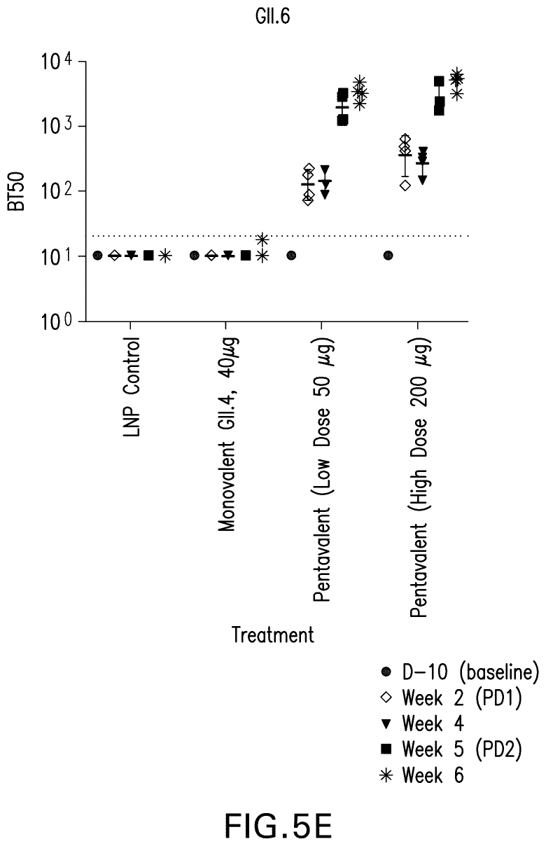

FIGS. 5A-5E depict the HBGA blocking titers of sera samples from animals vaccinated with the test vaccines. The vaccine groups tested are shown on the x-axis and BT50 against each of the five different genotype of norovirus is plotted. The dotted line at 20 indicates the limit of detection. A value of 10 indicates a lack of blocking, even at the lowest dilution tested. Inoculations at two dose levels of 50 μg or 200 μg of the pentavalent vaccine candidate elicited comparable and robust HBGA blocking antibody titers specific to the five norovirus genotypes GI.1, GII.2, GII.3, GII.4, and GII.6. Pentavalent vaccine candidates and monovalent GII.4 vaccine elicited comparable HBGA blocking antibody titers against GII.4. The second dose at both dose levels boosted antibody titers.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

1. A messenger ribonucleic acid (mRNA) polynucleotide encoding a norovirus VP1 polypeptide, wherein the mRNA polynucleotide is selected from the group consisting of SEQ ID NOs: 1-5 and sequences having at least 80% identity thereto, and wherein at least one uridine residue in the mRNA polynucleotide is replaced with N1-methylpseudouridine.

2. (canceled)

3. The mRNA polynucleotide of claim 1, wherein the mRNA polynucleotide further comprises a heterologous 5′ untranslated region (UTR) and a heterologous 3′ UTR.

4. The mRNA polynucleotide of claim 3, wherein the 5′ UTR comprises the polynucleotide sequence of SEQ ID NO:6.

5. The mRNA polynucleotide of claim 4, wherein the 5′ UTR further comprises the 5′ cap structure N7-Methyl-G (3′OMe) ppp.

6. The mRNA polynucleotide of claim 3, wherein the 3′ UTR comprises a poly(A) sequence and a poly(C) sequence.

7. The mRNA polynucleotide of claim 6, wherein the poly(A) sequence comprises from 10 to 200 adenosine nucleotides, and the poly(C) sequence comprises from 10 to 200 cytosine nucleotides.

8. The mRNA polynucleotide of claim 6, wherein the poly(A) sequence comprises from 10 to 100 adenosine nucleotides.

9. (canceled)

10. The mRNA polynucleotide of claim 6, wherein the poly(A) sequence comprises from 50 to 70 adenosine nucleotides.

11. The mRNA polynucleotide of claim 6, wherein the poly(C) sequence comprises from 10 to 100 cytosine nucleotides.

12.-14. (canceled)

15. The mRNA polynucleotide of claim 7, wherein the poly(A) sequence comprises 80 adenine nucleotides.

16. The mRNA polynucleotide of claim 3, wherein the 3′ UTR comprises the polynucleotide sequence of SEQ ID NO:7 or SEQ ID NO: 8.

17. The mRNA polynucleotide of claim 10, wherein the 3′ UTR comprises the polynucleotide sequence of SEQ ID NO:7.

18. The mRNA polynucleotide of claim 10, wherein the 3′ UTR comprises the polynucleotide sequence of SEQ ID NO:8.

19. The mRNA polynucleotide of claim 1, wherein the mRNA polynucleotide comprises, from the 5′ to the 3′ direction:
a 5′ UTR comprising the polynucleotide sequence of SEQ ID NO:6;
a polynucleotide encoding a norovirus VP1 polypeptide, wherein the polynucleotide is selected from the group consisting of SEQ ID NOs: 1-5 and sequences having at least 80% identity thereto; and
a 3′ UTR comprising the polynucleotide sequence of SEQ ID NO:7 or SEQ ID NO: 8; and
wherein the 5′ UTR further comprises the 5′ cap structure N7-Methyl-G (3′OMe) ppp, and all uridine residues in the polynucleotide encoding the norovirus VP1 polypeptide are replaced with N1-methylpseudouridine.

20. (canceled)

21. The mRNA polynucleotide of claim 1, wherein the mRNA polynucleotide comprises at least one histone stem-loop structure.

22. The mRNA polynucleotide of claim 1 wherein at least 50% of the uridine residues in the mRNA polynucleotide are replaced with N1-methylpseudouridine.

23.-24. (canceled)

25. The mRNA polynucleotide of claim 1 wherein at least 95% of the uridine residues in the mRNA polynucleotide are replaced with N1-methylpseudouridine.

26.-27. (canceled)

28. A composition comprising at least one mRNA polynucleotide encoding a norovirus VP1 polypeptide according to claim 1 and a pharmaceutically acceptable carrier.

29.-32. (canceled)

33. A composition comprising five mRNA polynucleotides encoding norovirus VP1 polypeptides and a pharmaceutically acceptable carrier, wherein each of the five mRNA polynucleotides comprises, from the 5′ to the 3′ direction:
a 5′ UTR comprising the polynucleotide sequence of SEQ ID NO:6;
a polynucleotide encoding a norovirus VP1 polypeptide, wherein the polynucleotide is selected from the group consisting of SEQ ID NOs: 1-5 and sequences having at least 80% 70% identity thereto; and
a 3′ UTR comprising the polynucleotide sequence of SEQ ID NO:7 or SEQ ID NO: 8; and
wherein the 5′ UTR further comprises the 5′ cap structure N7-Methyl-G (3′OMe) ppp, and
wherein the composition comprises each of SEQ ID NOs: 1-5, and sequences having at least 80% identity thereto, wherein in each of SEQ ID NOs: 1-5, and sequences having at least 80% identity thereto, all uridine residues in the five mRNA polynucleotide are replaced with N1-methylpseudouridine.

34.-35. (canceled)

36. A composition comprising at least one mRNA polynucleotide encoding a norovirus VP1 polypeptide and a pharmaceutically acceptable carrier, wherein the mRNA polynucleotide is selected from the group consisting of SEQ ID NOs: 1-5 and sequences having at least 80% thereto, and wherein each mRNA polynucleotide is encapsulated in a lipid nanoparticle (LNP) comprising one or more cationic or polycationic compounds.

37.-58. (canceled)

59. A vaccine comprising the mRNA polynucleotide according to claim 1.

60.-68. (canceled)

69. A method of treating or preventing a disorder, wherein the method comprises administering to a subject in need thereof an effective amount of the mRNA polynucleotide according to claim 1.

70. (canceled)

71. A vector comprising one or more nucleic acids encoding the mRNA polynucleotide according to claim 1.

72. A host cell comprising the mRNA polynucleotide according to claim 1.

73. A polypeptide encoded by the mRNA polynucleotide according to claim 1.

74. A method of making a virus-like particle (VLP) comprising one or more norovirus VP1 polypeptide, wherein the method comprises transcribing the mRNA polynucleotide according to claim 1 in a recombinant nucleic acid expression system under conditions suitable for assembly of the expressed one or more norovirus VP1 polypeptide into a VLP.

75. (canceled)
